SEQUENCE LISTING (55 sequences)

SEQ ID NO: 1 - DNA, 20 bp, Artificial sequence
  Synthetic
gcctcctgcaccaccaactg

SEQ ID NO: 2 - DNA, 23 bp, Artificial sequence
  Synthetic
cgacgcctgcttcaccaccttct

SEQ ID NO: 3 - DNA, 20 bp, Artificial sequence
  Synthetic
gcacatctgcttcaacagga

SEQ ID NO: 4 - DNA, 20 bp, Artificial sequence
  Synthetic
ccaaacataaatgccccatc

SEQ ID NO: 5 - DNA, 20 bp, Artificial sequence
  Synthetic
cctgtcttggacgagaccat

SEQ ID NO: 6 - DNA, 20 bp, Artificial sequence
  Synthetic
aaggcctcctaagctccaag

SEQ ID NO: 7 - DNA, 21 bp, Artificial sequence
  Synthetic
aagcctgcctcatccttgacc

SEQ ID NO: 8 - DNA, 20 bp, Artificial sequence
  Synthetic
gacgagcctctccttcacgg

SEQ ID NO: 9 - DNA, 20 bp, Artificial sequence
  Synthetic
ttcggccgctattagcttag

SEQ ID NO: 10 - DNA, 20 bp, Artificial sequence
  Synthetic
gacgggggaataatcaacct

SEQ ID NO: 11 - DNA, 20 bp, Artificial sequence
  Synthetic
cgtgccaatcttgaggtttt

SEQ ID NO: 12 - DNA, 20 bp, Artificial sequence
  Synthetic
cggaggcgtggttaaataaa

SEQ ID NO: 13 - DNA, 19 bp, Artificial sequence
  Synthetic
cctccctgaccgttcccag

SEQ ID NO: 14 - DNA, 21 bp, Artificial sequence
  Synthetic
gcagtttgagaccgccacatc

SEQ ID NO: 15 - DNA, 20 bp, Artificial sequence
  Synthetic
tggcgagtttgcgtcctcag

SEQ ID NO: 16 - DNA, 23 bp, Artificial sequence
  Synthetic
cgtccagtgtcacgatttcttgg

SEQ ID NO: 17 - DNA, 20 bp, Artificial sequence
  Synthetic
tcgcgttttcgaaagaaggc

SEQ ID NO: 18 - DNA, 20 bp, Artificial sequence
  Synthetic
tctacgtaatgacggaccca

SEQ ID NO: 19 - DNA, 17 bp, Artificial sequence
  Synthetic
ctttggcgcggatcctc

SEQ ID NO: 20 - DNA, 25 bp, Artificial sequence
  Synthetic
agtccttcttggctagtctgttgac

SEQ ID NO: 21 - DNA, 22 bp, Artificial sequence
  Synthetic
catcaagaagctgctggcggcc

SEQ ID NO: 22 - DNA, 22 bp, Artificial sequence
  Synthetic
ggaattgatttgggagagcatc

SEQ ID NO: 23 - DNA, 26 bp, Artificial sequence
  Synthetic
caggtcctggctctacaatttactaa

SEQ ID NO: 24 - DNA, 28 bp, Artificial sequence
  Synthetic
agtgtgactgggcagatcatccaccttc

SEQ ID NO: 25 - DNA, 21 bp, Artificial sequence
  Synthetic
gttgggacatgttcctgacgg

SEQ ID NO: 26 - DNA, 21 bp, Artificial sequence
  Synthetic
ctccctccatccctatgctgc

SEQ ID NO: 27 - DNA, 20 bp, Artificial sequence
  Synthetic
gcgagaggtgtgtcagccaa

SEQ ID NO: 28 - DNA, 19 bp, Artificial sequence
  Synthetic
tgggagccaaagaggcagg

SEQ ID NO: 29 - DNA, 21 bp, Artificial sequence
  Synthetic
tccagtggggtaaatgcacct

SEQ ID NO: 30 - DNA, 20 bp, Artificial sequence
  Synthetic
aagatagcatggccgtgggg

SEQ ID NO: 31 - DNA, 20 bp, Artificial sequence
  Synthetic
aggaagttgagggcgttctg

SEQ ID NO: 32 - DNA, 20 bp, Artificial sequence
  Synthetic
ggcccacatttgagaggtca

SEQ ID NO: 33 - DNA, 55 bp, Artificial sequence
  Synthetic
caagcagaagacggcatacgagatcgagtaatgtgactggagttcagacgtgtgc

SEQ ID NO: 34 - DNA, 55 bp, Artificial sequence
  Synthetic
caagcagaagacggcatacgagattctccggagtgactggagttcagacgtgtgc

SEQ ID NO: 35 - DNA, 25 bp, Artificial sequence
  Synthetic
gtgactggagttcagacgtgtgctc

SEQ ID NO: 36 - DNA, 49 bp, Artificial sequence
  Synthetic
aatgatacggcgaccaccgagatctacactctttccctacacgacgctc

SEQ ID NO: 37 - protein, 11 aa, Artificial sequence
  Synthetic
CAFFSWGKLQF

SEQ ID NO: 38 - protein, 15 aa, Artificial sequence
  Synthetic
CASSQSPGTGVGYTF

SEQ ID NO: 39 - protein, 16 aa, Artificial sequence
  Synthetic
CAASKEGGGSQGNLIF

SEQ ID NO: 40 - protein, 16 aa, Artificial sequence
  Synthetic
CSARDRGEHPPNDQFF

SEQ ID NO: 41 - protein, 14 aa, Artificial sequence
  Synthetic
CAEKGWDSSYKLIF

SEQ ID NO: 42 - protein, 13 aa, Artificial sequence
  Synthetic
CAWVDGVLDGYTF

SEQ ID NO: 43 - protein, 12 aa, Artificial sequence
  Synthetic
CAASEGGQKLLF

SEQ ID NO: 44 - protein, 17 aa, Artificial sequence
  Synthetic
CASSAPNSDSAKNIQYF

SEQ ID NO: 45 - protein, 15 aa, Artificial sequence
  Synthetic
CILRDGVGYGNKLVF

SEQ ID NO: 46 - protein, 14 aa, Artificial sequence
  Synthetic
CASEDRGGTDTQYF

SEQ ID NO: 47 - protein, 15 aa, Artificial sequence
  Synthetic
CAMREGSGVGTSYGK

SEQ ID NO: 48 - protein, 15 aa, Artificial sequence
  Synthetic
CSAAGTSGSGETQYF

SEQ ID NO: 49 - protein, 11 aa, Artificial sequence
  Synthetic
EPLPQGQLTAY

SEQ ID NO: 50 - protein, 11 aa, Artificial sequence
  Synthetic
LPQGQLTAYHV

SEQ ID NO: 51 - protein, 9 aa, Artificial sequence
  Synthetic
DSELEIKRY

SEQ ID NO: 52 - protein, 8 aa, Artificial sequence
  Synthetic
IKRYKNRV

SEQ ID NO: 53 - protein, 8 aa, Artificial sequence
  Synthetic
RYKNRVAS

SEQ ID NO: 54 - protein, 9 aa, Artificial sequence
  Synthetic
DSELEIKRY

SEQ ID NO: 55 - protein, 9 aa, Artificial sequence
  Synthetic
CDSELEIKR

(19) United States
(12) Patent Application Publication
(10) Pub. No.: US 2024/0325539 A1
(43) Pub. Date: Oct. 3, 2024

(54) METHODS AND COMPOSITIONS FOR TREATING EPSTEIN BARR VIRUS-ASSOCIATED CANCER

(71) Applicants: The Chancellor, Masters and Scholars of the University of Oxford, Oxford (GB); Imperial College Innovations Limited, London (GB)

(72) Inventors: Xin Lu, Headington, Oxford (GB); Xiao-Ning Xu, Headington, Oxford (GB); Nadia Khan, Greater London (GB); Stefanie Zentelis, Headington, Oxford (GB)

(73) Assignees: The Chancellor, Masters and Scholars of the University of Oxford, Oxford (GB); Imperial College Innovations Limited, London (GB)

(21) Appl. No.: 18/249,769

(22) PCT Filed: Oct. 20, 2021
(86) PCT No.: PCT/EP21/79137

§ 371 Date: Apr. 20, 2023

Related U.S. Application Data
(60) Provisional application No. 63/094,104, filed on Oct. 20, 2020.

(51) Int. Cl. A61K 39/00; A61K 31/136; A61K 31/4406; A61K 31/454; A61K 31/473; A61K 31/496; A61K 31/506; A61K 31/704; A61K 31/7048; C07K 14/725; C07K 16/08

(52) U.S. Cl. CPC A61K 39/464838; A61K 31/136; A61K 31/4406; A61K 31/454; A61K 31/473; A61K 31/496; A61K 31/506; A61K 31/704; A61K 31/7048; A61K 39/4611; A61K 39/4632; C07K 14/7051; C07K 16/085; A61K 2239/39; A61K 2239/51

(57) ABSTRACT

This disclosure describes a novel “kick and kill” strategy as an effective cancer therapy for treating virus-associated cancers. In particular, this disclosure provides a method of reactivating a latent Epstein-Barr virus (EBV) in a cell infected with the EBV. Also provided are a method of eliciting or enhancing an immune response against an EBV-positive cancer cell in a subject infected with the EBV and a method of treating a subject having cancer associated with EBV infection.

### CROSS-REFERENCE TO RELATED APPLICATIONS

The present application is a National Stage Application under 35 U.S.C. § 371 of International Application No. PCT/EP2021/079137 filed Oct. 20, 2021, which claims priority under 35 U.S.C. § 119 (e) to U.S. Provisional Patent Application No. 63/094,104, filed Oct. 20, 2020. The entire contents of these applications are incorporated herein by reference in their entirety.

### SEQUENCE LISTING

This application contains a Sequence Listing which has been submitted electronically in xml format and is hereby incorporated by reference in its entirety. Said xml copy, created on Apr. 18, 2023, is named “Seq Listing 084276.00360_ST25.xml” and is 11,264 bytes in size.

### FIELD OF THE INVENTION

This invention relates to methods and compositions for treating Epstein-Barr virus (EBV)-associated diseases such as EBV-positive cancer.

### BACKGROUND OF THE INVENTION

The Epstein-Barr virus (EBV), also called human herpesvirus 4 (HHV-4), is the cause of infectious mononucleosis (glandular fever) and associated with particular forms of cancer, such as Hodgkin's lymphoma, Burkitt's lymphoma, nasopharyngeal carcinoma, and conditions associated with human immunodeficiency virus (HIV), e.g., hairy leukoplakia and central nervous system lymphomas. Infection with EBV is also associated with a higher risk of certain autoimmune diseases, especially dermatomyositis, systemic lupus erythematosus, rheumatoid arthritis, Sjogren's syndrome, and multiple sclerosis. EBV infects more than 90% of the global population, but it exists in a latent state in most infected individuals and escapes immune surveillance. Each year approximately 210,000 cancer cases are attributed to EBV, such as B-cell malignancies, nasopharyngeal cancer (NPC), gastric cancer (GC), and some rare T/NK cell lymphomas, leukemias, and leiomyosarcomas. The epithelial cancers NPC and GC are the major group of EBV-associated malignancies with high mortalities, of which EBV+GC is the largest category of EBV-cancer, with more than 80,000 cases per year.

Thus, there is a pressing need for developing methods and agents to prevent or treat EBV-associated diseases, such as EBV-positive cancer.

### SUMMARY OF THE INVENTION

This disclosure addresses the need mentioned above in a number of aspects. In one aspect, this disclosure provides a method of reactivating a latent Epstein-Barr virus (EBV) in a cell infected with the EBV. The method comprises contacting the cell with a benzamide-based histone deacetylase (HDAC) inhibitor, wherein the benzamide-based HDAC inhibitor increases a level of expression or activity of an EBV-associated protein (e.g., viral transcriptional factor Zta) in the EBV-positive cancer cell. In some embodiments, the method further comprises contacting the cell with a second agent (e.g., one or more additional latency reactivation agents).

In another aspect, this disclosure also provides a method of eliciting or enhancing an immune response against an EBV-positive cancer cell in a subject infected with the EBV. The method comprises administering to the subject an effective amount of a benzamide-based HDAC inhibitor, wherein the benzamide-based HDAC inhibitor increases a level of expression or activity of an EBV-associated protein in the EBV-positive cancer cell.

In another aspect, this disclosure further provides a method of killing an EBV-positive cancer cell in a subject infected with the EBV. The method comprises administering to the subject an effective amount of a benzamide-based HDAC inhibitor, wherein the benzamide-based HDAC inhibitor increases a level of expression or activity of an EBV-associated protein in the EBV-positive cancer cell.

In another aspect, this disclosure additionally provides a method of treating a subject having cancer associated with EBV infection. The method comprises administering to the subject an effective amount of a benzamide-based HDAC inhibitor, wherein the benzamide-based HDAC inhibitor increases a level of expression or activity of an EBV-associated protein in an EBV-positive cancer cell.

In some embodiments, the subject is a mammal (e.g., human). In some embodiments, the EBV-associated protein is transcription factor Zta. In some embodiments, the cell is an EBV-positive cancer cell. In some embodiments, the EBV-positive cancer cell is an EBV-positive gastric cancer cell. In some embodiments, the cancer is gastric cancer.

In some embodiments, the benzamide-based HDAC inhibitor comprises any one of chidamide, CXD101, entinostat, mocetinostat, and combinations thereof. In some embodiments, the benzamide-based HDAC inhibitor comprises chidamide.

In some embodiments, the method further comprises administering to the subject a second agent. In some embodiments, the second agent comprises a topoisomerase inhibitor (e.g., topoisomerase II inhibitor). In some embodiments, the topoisomerase inhibitor comprises any one of epirubicin, doxorubicin, mitoxantrone, amonafide, teniposide, and combinations thereof. In some embodiments, the topoisomerase inhibitor comprises epirubicin.

In some embodiments, the second agent comprises an Mdm2 inhibitor. In some embodiments, the Mdm2 inhibitor comprises nutlin-3a, HDM201, or a combination thereof.

In some embodiments, the second agent comprises an anti-cancer agent (e.g., an immune checkpoint inhibitor).

In some embodiments, the method further comprises administering to the subject a lymphocyte transduced with a recombinant T cell receptor (TCR). In some embodiments, the recombinant TCR comprises a Zta-specific TCR. In some embodiments, the recombinant TCR-transduced lymphocyte shows reactivity to the transcriptional factor Zta or a fragment thereof. In some embodiments, the recombinant TCR binds specifically to the transcriptional factor Zta or a fragment thereof. In some embodiments, the recombinant TCR comprises an amino acid sequence having at least 90% sequence identity to an amino acid sequence of SEQ ID NOs: 37-48 or comprises an amino acid sequence of SEQ ID NOs: 37-48. In some embodiments, the recombinant TCR binds specifically to an antigen comprising an amino acid sequence of SEQ ID NOs: 49-53 and 55.

In some embodiments, the lymphocyte comprises a CD8+ T cell or a CD4+ T cell.

In some embodiments, the method further comprises administering to the subject an EBV vaccine and optionally an adjuvant.

In some embodiments, the benzamide-based HDAC inhibitor is administered orally, topically, intravenously, intraperitoneally, intramuscularly, intralesionally, intrathecally, intranasally, subcutaneously, parenterally, transmucosally, sublingually, in controlled release, in delayed release, or as a suppository.

In some embodiments, the second agent is administered to the subject before, after, or concurrently with the benzamide-based HDAC inhibitor.

In another aspect, this disclosure also provides a composition for eliciting or enhancing an immune response against an EBV-positive cancer cell in a subject infected with the EBV. In some embodiments, the composition comprises: (i) benzamide-based HDAC inhibitor; (ii) a topoisomerase inhibitor (e.g., topoisomerase II inhibitor) or an Mdm2 inhibitor; and (iii) optionally a pharmaceutically acceptable carrier.

In some embodiments, the benzamide-based HDAC inhibitor comprises any one of chidamide, CXD101, entinostat, mocetinostat, and combinations thereof. In some embodiments, the topoisomerase inhibitor comprises any one of epirubicin, doxorubicin, mitoxantrone, amonafide, teniposide, and combinations thereof. In some embodiments, the Mdm2 inhibitor comprises nutlin-3a, HDM201, or a combination thereof. In some embodiments, the composition comprises chidamide, epirubicin, and optionally the pharmaceutically acceptable carrier.

In some embodiments, the composition is an immunogenic composition optionally comprising a pharmaceutically acceptable diluent, vehicle, one or more immunological adjuvants, or combinations thereof.

In another aspect, this disclosure additionally provides a kit for eliciting or enhancing an immune response against an EBV-positive cancer cell in a subject infected with the EBV. In some embodiments, the kit comprises: (i) benzamide-based HDAC inhibitor; (ii) a topoisomerase inhibitor (e.g., topoisomerase II inhibitor) or an Mdm2 inhibitor; and (iii) optionally a pharmaceutically acceptable carrier. In some embodiments, the benzamide-based HDAC inhibitor comprises any one of chidamide, CXD101, entinostat, mocetinostat, and combinations thereof. In some embodiments, the topoisomerase inhibitor comprises any one of epirubicin, doxorubicin, mitoxantrone, amonafide, teniposide, and combinations thereof. In some embodiments, the Mdm2 inhibitor comprises nutlin-3a, HDM201, or a combination thereof. In some embodiments, the kit comprises chidamide, epirubicin, and optionally the pharmaceutically acceptable carrier.

In yet another aspect, this disclosure provides a TCR or antigen-binding fragment thereof comprising an amino acid sequence having at least 90% sequence identity to an amino acid sequence of SEQ ID NOs: 37-48 or comprising an amino acid sequence of SEQ ID NOs: 37-48.

In some embodiments, the TCR or antigen-binding fragment thereof binds specifically to the transcriptional factor Zta or a fragment thereof. In some embodiments, the recombinant TCR binds specifically to an antigen comprising an amino acid sequence of SEQ ID NOs: 49-53 and 55.

In yet another aspect, this disclosure additionally provides a nucleic acid comprising a polynucleotide sequence that encodes a TCR or antigen-binding fragment thereof described herein. Also provided is a vector comprising a nucleic acid described herein. In some embodiments, the vector comprises a retroviral vector or a lentiviral vector. Additionally provided is a cell comprising a nucleic acid or a vector, as described herein. In some embodiments, the cell comprises an immune cell. In some embodiments, the immune cell comprises a lymphocyte (e.g., T cell or a natural killer (NK) cell).

Also within the scope of this disclosure is a composition comprising a TCR or antigen-binding fragment thereof, a nucleic acid, a vector, or a cell, as described herein.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A, 1B, 1C, 1D, and 1E (collectively “FIG. 1”) are a set of diagrams showing that benzamide-based histone deacetylase (HDAC) inhibitor (HDACi) can reactivate EBV. FIG. 1A shows percentage of Zta positive YCCEL1 cells after treatment with epigenetic inhibitors at high concentrations (shown for each drug). In the upper panel, representative images of confocal immunofluorescence analysis of Zta expression in YCCEL1 cells after treatment with chidamide or CXD101. Values shown are mean+SD, ** P<0.01 (compared with DMSO) by one-way ANOVA test, n=3 (biological replicates). FIG. 1B shows percentage of Zta positive YCCEL1 cells after treatment with HDACi at three different concentrations. Values shown are mean+SD, * P<0.05, ** P<0.01 (compared with DMSO), by one-way ANOVA test, n=3 (biological replicates). FIG. 1C shows the results of the Western blot for Zta and total acetylation of histone 3 in YCCEL1 cells treated with the intermediate concentrations of the drugs from FIG. 1B. The bar plot shows Zta band intensity normalized over the total acetylation of histone 3 and β-actin band intensity (fold change over DMSO). Values shown are mean+SD, n=3 (biological replicates). FIG. 1D (left panel) shows percentage of Zta-positive AGS-EBV, YCCEL1, and SNU719 cells after treatment with 5 μM SAHA, 5 μM chidamide, or 2.5 μM CXD101. Values shown are mean+SD, * P<0.05, ** P<0.01 (compared with DMSO) by one-way ANOVA test, n=3 (biological replicates). FIG. 1D (right panel) shows the results of the Western blot analysis of Zta in the same samples; β-actin as a loading control. FIG. 1E (left panel) shows the results of the Western blot analysis of Zta and total acetylation of histone 3 in YCCEL1, Namalwa, Raji, Akata, and PD-LCL cells treated with 5 μM chidamide, 2.5 μM CXD101, or 2.5 nM romidepsin. β-actin as a loading control. Lysate of YCCEL1 cells treated with 5 μM chidamide was used as a positive control for Zta staining. FIG. 1E (right panel) shows Zta band intensity normalized over the total acetylation of histone 3 and β-actin band intensity (fold change over DMSO). Values shown are mean+SD, n=2 or n=3 (biological replicates).

FIGS. 2A, 2B, 2C, 2D, 2E, and 2F (collectively “FIG. 2”) are a set of diagrams showing topoisomerase inhibitors synergize with HDACi chidamide to reactivate EBV with high efficiency. FIG. 2A shows a schematic representation of the drug libraries used for screening. FIG. 2B are dot plots showing distribution of Z-scores of Zta expression in YCCEL1 cells treated with drugs alone or in combination with low dose (2.5 μM) chidamide. Each dot represents a single drug from the library. Cytostatic drugs are indicated. FIG. 2C shows fold enrichment of drug classes from the library based on number of hits. The number of hits and the total number of drugs in a class are shown next to the class name. FIG. 2D (left panel) is a plot showing distribution of Z-score of Zta expression for cytostatic drugs, with or without chidamide. FIG. 2D (right panel) shows the drugs ordered according to subclasses. FIG. 2E is a heatmap showing Z-scores of Zta expression and percentage of remaining cells after treatment with 9 topoisomerase inhibitors, with or without chidamide. Topoisomerase (Topo) class (I or II) is indicated. FIG. 2F (left panel) shows percentage of Zta positive YCCEL1 cells after treatment with topoisomerase inhibitors from FIG. 2E, alone or in combination with chidamide after dose optimization. Values shown are mean+SD, ** P<0.01 by two-way ANOVA test, n=3 (biological replicates). FIG. 2F (right panel) shows the results of the Western blot analysis of p53 and Zta in the same samples; β-actin as a loading control.

FIGS. 3A and 3B (collectively “FIG. 3”) are a set of diagrams showing that enhanced p53 activity synergizes with HDACi to induce EBV latency reversal. FIG. 3A shows percentage of Zta positive YCCEL1 cells after treatment with chemotherapeutics or Mdm2 inhibitors, alone or combined with 2.5 μM chidamide. Values shown are mean+SD, * P<0.05, ** P<0.01 by two-way ANOVA test, n=3 (biological replicates). FIG. 3B shows the results of the Western blot analysis of p53 and Zta levels upon treatment with different HDACi combined with nutlin-3a in AGS-EBV, YCCEL1, and SNU719 cell lines. β-actin as a loading control.

FIGS. 4A, 4B, 4C, 4D, 4E, and 4F (collectively “FIG. 4”) are a set of diagrams showing that wild type p53 is required for EBV latency reversal in EBV-positive epithelial cancer cells. FIG. 4A shows percentage of Zta (upper) and positive YCCEL1 cells and p53 fluorescence intensity (relative to DMSO-treated scramble control) after treatment with epirubicin or HDM201 with or without chidamide, with siRNAs silencing p53 expression (sip53). The values shown are mean+SD. Representative images show expression levels of Zta and p53, under treatment conditions indicated. FIG. 4B shows the results of the Western blot analysis of p53 and Zta after EBV reactivation with chidamide or CXD101 combined with nutlin-3a, with or without p53 siRNA, in YCCEL1, C666.1, and NCC24 cells. FIG. 4C (left panel) shows the results of the Western blot analysis of p53, HA, and Zta after EBV reactivation with chidamide, with or without overexpression of wild-type p53-HA in NCC24 cells. FIG. 4C (right panel) shows the results of the immunofluorescence analysis of Zta in NCC24 cells, transfected with p53-HA, and treated with either DMSO or chidamide. FIG. 4D shows the results of the Western blot analysis of p53, HA, and Zta after EBV reactivation with chidamide, with or without overexpression of wild-type p53-HA in YCCEL1 cells. FIG. 4E (left panel) is a schematic showing the location of two putative p53 binding sites in promoter and enhancer regions of Zta (BZLF1) locus. FIG. 4E (right panel) shows the results of the ChIP-qPCR analysis of p53 binding to putative binding sites in promoter and enhancer regions of Zta (BZLF1) locus. p21 ((DKN1A) promoter and KRT14 promoter were used as positive and negative controls of p53 binding to host chromatin, respectively. Values shown are mean+SD, * P<0.05, ** P<0.01 (compared with anti-p53 ChIP in DMSO-treated control) by one-way ANOVA test, n=3 (biological replicates).

FIGS. 5A, 5B, 5C, 5D, 5E, 5F, 5G, 5H, and 5I (collectively “FIG. 5”) are a set of diagrams showing the results of single-cell sequencing that reveal distinct subtypes of reactivation induced by latency reversal agents. FIG. 5A shows UMAP visualization depicting unsupervised transcriptome clustering of YCCEL1 cells after treatment with DMSO, epirubicin, or HDM201 alone or in combination with chidamide. Each cell is indicated by the treatment used. Clusters predominantly composed of cells treated with drug combinations are denoted as C1, C2, C3, and C4. FIG. 5B shows expression levels of example EBV genes from different phases of the lytic cycle, with zoomed in region above. For each gene, UMAP plots are overlaid with a scale representing log-normalized UMI count levels for that gene. The rightmost facet displays the treatment conditions for comparison. Regions corresponding with the subclusters identified within C1 (C1A and C1B) are labeled. FIG. 5C shows the results of the pseudotime and cell trajectory analysis overlaid on the UMAP visualization. Each numbered point represents a branch node or end node. Solid lines represent how the nodes are connected in the constructed trajectories. The cells are indicated by pseudotime, representing how far each cell has progressed along the trajectory branches away from the root node (yellow being the latest in pseudotime). FIG. 5D shows levels of spliced (left) and unspliced (middle) BZLF1 (Zta) RNA in each cell. The right panel shows the residuals from a model fit across the dataset to compare the unspliced and spliced BZLF1 levels within each cell; the residual represents the difference between the observed unspliced expression level and the expected level from this model. Higher residuals (greater unspliced expression than expected) have a deeper color, while lower residuals (lower unspliced expression than expected) have a deeper color and are indicated with a circle. Subclusters C1A and C1B are shown. FIG. 5E (left panel) shows UMAP visualization from FIG. 5A re-colored according to cell cluster population type. Names are based on the EBV reactivation state. FIG. 5E (right panel) shows a Sankey diagram showing the contribution of each treatment condition to each of the major EBV reactivation states. FIG. 5F shows mRNA expression levels of EBV genes analyzed for each of the cluster types depicted in FIG. 5E. For each point, the size corresponds to the percentage of cells within that cluster that express any detectable level of that gene, and the average expression level of that gene within that cluster is also indicated. FIG. 5G is a Violin plot showing expression of EBV metafeature BGLF3/BGLF3.5/BGLF4 in the C1 cluster in cells treated with chidamide combined with epirubicin or chidamide combined with HDM201. FIG. 5H shows the results of the RT-qPCR analysis of BGLF4 expression relative to DMSO. YCCEL1 cells were treated with epirubicin or Nutlin-3A alone or in combination with chidamide for 48 h. Values shown are mean+SD, ** P<0.01 (compared with DMSO) by one-way ANOVA test, n=3 (biological replicates). FIG. 5I shows the results of the qPCR analysis of EBV DNA copy number relative to DMSO. YCCEL1 cells were treated with epirubicin or Nutlin-3A alone or in combination with chidamide for 48 h. Values shown are mean+SD, ** P<0.01 (compared with DMSO) by one-way ANOVA test, n=3 (biological replicates).

FIGS. 6A, 6B, and 6C (collectively “FIG. 6”) are a set of diagrams showing that epirubicin combined with chidamide induces abortive EBV reactivation by enhancing Zta SUMOylation. FIG. 6A (left panel) shows mRNA expression level of genes related to the SUMOylation pathway, analyzed for each of the cluster types depicted in FIG. 5E. For each point, the size corresponds to the percentage of cells within that cluster that express any detectable level of that gene, and the average expression level of that gene within that cluster is indicated. FIG. 6A (right panel) are the UMAP plots shown on the treatment condition or SAE1 log-normalized expression values. FIG. 6B shows the results of the Western blot of Zta and p53 in YCCEL1 cells treated with nutlin-3a or epirubicin alone or combined with chidamide. Shading and arrow highlight 47 kDa band. FIG. 6C shows the results of the Western blot of Zta expression in YCCEL1 cells treated with nutlin-3a or epirubicin combined with chidamide after overexpression of either wild-type or C603S mutant SENP1. Shading and arrow highlight 47 kDa band.

FIGS. 7A, 7B, 7C, and 7D (collectively “FIG. 7”) are a set of diagrams showing that EBV latency-reversed gastric cancer cells are killed by Zta-specific T cells. FIG. 7A shows the effect of HDACi on ex vivo T cell responses by IFNγ-ELISpot analysis of number of spot-forming units (SFU) per million cells after stimulation of PBMCs from a healthy donor (representative of three healthy donors) with EBV peptide pool in the presence of HDACi at different concentrations. Data are shown as a mean percentage of SFU relative to untreated sample±SD, n=3 (biological replicates). FIG. 7B shows cytokine secretion of primary T cells expressing HLA-matched (TCR4) or HLA-mismatched (TCR9) Zta-specific TCRs after incubation of AGS-EBV cells at an Effector:Target ratio of 10:1 for 24 hours. AGS-EBV cells were treated with SAHA, chidamide, or CXD101 for 48 hours prior to incubation with T cells. FIG. 7C is a representative image showing green fluorescence of GFP-positive AGS-EBV cells and red fluorescence of Cytotox Red apoptotic marker. AGS-EBV cells are identified as GFP-high compared with GFP-low T cells. On the right, an enlarged image of the boxed area is shown. Fluorescence marks apoptotic cells (Cytotox Red). FIG. 7D shows killing kinetics of AGS-EBV cells by primary T cells expressing Zta-specific HLA-matched TCR4 or HLA-mismatched TCR9. AGS-EBV cells were treated with SAHA, chidamide, or CXD101 prior to incubation with T cells as in FIG. 7B. Cell death was estimated by measuring the area of red fluorescence of the apoptosis marker as μm²/per image. Line plots show mean values of 4 replicates with shading representing SEM, * P<0.05, ** P<0.01 by two-way ANOVA test (compared to DMSO for each time point).

FIG. 8 is a schematic representation of a new approach for treating EBV-associated gastric cancer. A combination of HDAC inhibitors with topoisomerase II (TopoII) inhibitors or Mdm2 inhibitors leads to efficient reactivation of EBV in epithelial tumor cells characterized by high levels of immunogenic viral antigens (Zta), which can be targeted by cytotoxic T cells. While Mdm2 inhibitors combined with HDAC inhibitors induce a full lytic cycle and viral DNA replication, TopoII inhibitors combined with HDACi inhibitors only induce an abortive reactivation without increased viral replication but with sufficient levels of immunogenic Zta antigen.

FIGS. 9A, 9B, 9C, 9D, 9E, and 9F (collectively “FIG. 9”) are a set of diagrams showing the additional results related to FIG. 1 that benzamide-based HDACi can reactivate EBV. FIG. 9A shows percentage of Zta positive YCCEL1 cells after treatment with different types of epigenetic inhibitors at low concentrations. Values shown are mean+SD, * P<0.05, ** P<0.01 (compared with DMSO) by one-way ANOVA test, n=3 (biological replicates). FIG. 9B shows the chemical structures of hydroxamate- and benzamide-based HDACi. FIG. 9C shows YCCEL1 cell number after treatment with different types of HDACi at three different concentrations, relative to DMSO-treated samples. Values shown are mean+SD, n=3 (biological replicates). FIG. 9D shows the results of the Western blot for Zta and total acetylation of histone 3 in SNU719 cells treated with the lowest concentrations of the drugs. β-actin is used as a loading control. Bar plot shows Zta band intensity normalized over the total acetylation of histone 3 and β-actin band intensity (fold change over DMSO). Values shown are mean+SD, n=3 (biological replicates). FIG. 9E shows the results of the RT-qPCR analysis of expression of EBV lytic genes in AGS-EBV, YCCEL1, and SNU719 cells treated with 5 μM SAHA, 5 μM chidamide, or 2.5 μM CXD101. Values shown are mean+SD, n=3 (biological replicates). FIG. 9F (left panel) shows percentage of VCA positive AGS-EBV, SNU719, and YCCEL1 cells after treatment with 5 μM SAHA, 5 μM chidamide, or 2.5 μM CXD101. Values shown are mean+SD, * P<0.05 (compared with DMSO) by one-way ANOVA test, n=3 (biological replicates). FIG. 9F (right panel) shows representative images of confocal immunofluorescence analysis of VCA expression in YCCEL1 cells.

FIG. 10 shows the additional results related to FIG. 2 that topoisomerase inhibitors synergize with HDACi chidamide to reactivate EBV with high efficiency. Percentage of Zta positive YCCEL1 cells after treatment with increasing concentrations of selected topoisomerase inhibitors is shown. Circles indicate a reduction of cell number below 25% with respect to control. Optimal concentrations chosen for further experiments are highlighted in green. Values shown are mean+SD, * P<0.05 (compared with DMSO) by one-way ANOVA test, n=3 (biological replicates).

FIG. 11 shows the additional results related to FIG. 3 that enhanced p53 activity synergizes with HDACi to induce EBV latency reversal. The results of the Western blot analysis of p53 and Zta levels upon treatment with different HDACi combined with nutlin-3a in C666.1 cells are shown. β-actin as a loading control.

FIGS. 12A and 12B (collectively “FIG. 12”) are a set of diagrams showing the additional results related to FIG. 4 that wild type p53 is required for EBV latency reversal in EBV-positive epithelial cancer cells. FIG. 12A is a schematic showing the location identified p53 binding sites (b.s.) in the BZLF1 locus. The b.s. score is shown based on the sequence length, the fraction of each nucleotide within the sequence, and the length of the spaces between half-sites. FIG. 12B shows the position of identified p53 binding sites relative to the transcription start site (TSS) of BZLF1 gene and the sequences of binding sites.

FIGS. 13A, 13B, 13C, 13D, 13E, 13F, 13G, and 13H (collectively “FIG. 13”) are a set of diagrams showing the additional results of single-cell sequencing related to FIG. 5 that reveal distinct subtypes of reactivation induced by latency reversal agents. FIG. 13A shows UMAP visualization of clusters to show all eight cell clusters identified by Monocle 3. Shared clusters predominantly derived from combination treatments, assigned the names C1-C4, are labeled. FIG. 13B are diagrams showing examples of the gene annotation modification strategy employed for quantification of overlapping genes in the EBV genome. The BZLF1/BRLF1 locus is shown as an example of genes with resolvable overlap (FIG. 13B, left). In the top subplot, the locations of BZLF1 and BRLF1 are shown, with the read depth (negative strand) across all 48 hr samples displayed above. Coding regions unique to each gene, where gene quantification is possible with Cell Ranger, are boxed. Below, UMAP plots are indicated by log-normalized expression values to display the unique expression pattern of BZLF1 and BRLF1. The BDLF1/BDLF2/BDLF3 locus is shown as an example where gene overlap is unresolvable for the purposes of individual gene quantification (FIG. 13B, right). The top subplot again shows the read depth at this locus and the location of the 3 individual overlapping genes. No coding regions are unique for BDLF1 and BDLF2, and the majority of BDFL3 (including the 3′ end with the greatest level of detected expression) is non-unique. Below, the gene aggregate representing the union of these 3 individual genes is shown. The bottom subplot shows the expression pattern of this gene aggregate. FIG. 13C shows separate UMAP plots that were generated for the 24 hr (left) and 48 hr (right) timepoints, and log-normalized BZLF1 levels are overlaid. FIG. 13D shows phase portrait modeling the relationship between the spliced and unspliced BZLF1 levels. The residuals for each cell, representing the difference between the observed level of unspliced expression and the expected level of unspliced expression from this model, are overlaid on the UMAP embedding (FIG. 13D, left). Higher residuals (cells with greater unspliced expression than expected for a given level of spliced RNA, as during the induction of gene expression) are indicated with an arrow, while lower residuals (lower unspliced expression than expected) are indicated with an empty triangle. In the right panel, spliced and unspliced expression values are plotted for each cell, and a model is fit to show the expected abundance of unspliced BZLF1 for a given level of spliced BZLF1 (dotted line). FIG. 13E shows UMAP embedding indicated according to the treatment timepoint, separated into separate facets for 24 h and 48 h timepoints. FIG. 13F shows UMAP embedding indicated based on treatment, separated into separate facets for 24 h (left) and 48 h (right) timepoints. FIG. 13G shows UMAP embedding colored by treatment condition, with the three EBV reactivation states labeled (FIG. 13G, left). Pie charts showing the preference of reactivation subtype for combination treatments (FIG. 13G, right). For both Chidamide+HDM201 (top) and Chidamide+Epirubicin (bottom), cells attaining either full or abortive reactivation states were subsetted, and pie charts were created to show the proportion within these subsets that obtain either full or abortive reactivation states. FIG. 13H shows qPCR analysis of EBV DNA copy number relative to DMSO. YCCEL1 cells were treated with epirubicin (epi), doxorubicin (dox), mitoxantrone (mit), amonafide (amo), teniposide (ten), or Nutlin-3a alone or in combination with chidamide. Values shown are mean+SD, ** P<0.01 (compared with DMSO) by two-way ANOVA test, n=3 (biological replicates).

FIG. 14 shows the additional results related to FIG. 6 that epirubicin combined with chidamide induces abortive EBV reactivation by enhancing Zta SUMOylation. The results of the Western blot analysis of Zta and p53 levels in YCCEL1 cells treated with epirubicin (epi), doxorubicin (dox), mitoxantrone (mit), amonafide (amo), or teniposide (ten) alone or in combination with chidamide are shown. Shading and arrow highlight 47 kDa band, referred to in the text.

FIGS. 15A and 15B (collectively “FIG. 15”) are a set of diagrams showing the additional results related to FIG. 7 that EBV latency-reversed gastric cancer cells are killed by Zta-specific T cells. FIG. 15A shows percentage of Zta positive or live AGS-EBV cells after treatment with increasing doses of HDACi. FIG. 15B (left panel) shows the results of the FACS analysis of CD69 expression levels in SKW3 cells expressing HLA-matched Zta-specific TCR4 after incubation with AGS-EBV cells treated with Zta-peptide pool. FIG. 15B (right panel) shows secretion of cytokines after incubation of AGS-EBV cells with SKW3 cells expressing HLA-matched Zta-specific TCR4 after treatment with SAHA, chidamide, or CXD101 prior to incubation with T-cells. n=1.

FIGS. 16A, 16B, and 16C are a set of graphs showing that Zta-specific primary T cell clones kill HDACi-treated AGS_rEBV cells. AGS_rEBV were treated with HDACi (FIG. 16A: CXD101; FIG. 16B: Chidamide; FIG. 16C: SAHA) or DMSO for 48 hours before co-culturing with primary T cell clones for 24 hours. Cell death is represented as μM2/per image, which represents the area of cells with fluorescence overlap of very high GFP (EBV-positive gastric cells) with red apoptosis marker in each replicate. Data shown is the mean of 4 replicates from the same experiment.

### DETAILED DESCRIPTION OF THE INVENTION

This disclosure is based, at least in part, on unexpected discoveries that a combination of a benzamide-based histone deacetylase (HDAC) inhibitor and a topoisomerase II inhibitor (or an Mdm2 inhibitor) can efficiently reactivate expression of EBV-associated protein(s), such as viral transcriptional factor Zta, while minimizing the risk of uncontrolled EBV infection. Reactivated expression of EBV-associated protein(s) in EBV-infected cancer cells provides an opportunity to eliminate the EBV-infected cancer cells via T cell-mediated killing. Thus, this disclosure provides a novel “kick and kill” strategy as an effective cancer therapy for treating virus-associated cancers.

### A. METHOD OF TREATMENT

### a. Methods of Reactivating a Latent EBV in a Cell

In one aspect, this disclosure provides a method of reactivating a latent EBV in a cell infected with the EBV. The method comprises contacting the cell with a benzamide-based HDAC inhibitor, or a pharmaceutically acceptable prodrug, a pharmaceutically active metabolite, or a pharmaceutically acceptable salt thereof, wherein the benzamide-based HDAC inhibitor increases a level of expression or activity of an EBV-associated protein in the EBV-positive cancer cell. In some embodiments, the benzamide-based HDAC inhibitor increases a level of expression or activity of transcription factor Zta in the cell.

Zta, also known as ZEBRA (BamHI Z Epstein-Barr virus replication activator), BZLF1 (BamHI Z fragment leftward open reading frame 1), or EB1, is an immediate-early viral gene of the Epstein-Barr virus (EBV) of the Herpes Virus Family, which induces cancers and infects primarily the B-cells of 95% of the human population. This gene (along with others) produces the expression of other EBV genes in other stages of disease progression, and is involved in converting the virus from the latent to the lytic form.

In some embodiments, the cell is an EBV-positive cancer cell. In some embodiments, the EBV-positive cancer cell is an EBV-positive gastric cancer cell.

The term “latent” or “latency” refers to a state of EBV in the host subject during which there is little if any viral replication and the subject is not infectious or contagious. At the latent state, the virus does not typically cause illness or symptoms. “Latency” also refers to as “latent infection,” which may occur in a different cell type from that of the initial/primary EBV infection.

The term “reactivation,” when used in reference to EBV, refers to activation of EBV in the host subject following a period of latency. Reactivation is associated with increased viral replication and proliferation in an EBV-infected host subject.

The terms “activate,” “increased,” “increase” or “enhance” are all used herein to generally mean an increase by a statically significant amount; for the avoidance of any doubt, the terms “increased,” “increase” or “enhance” or “activate” means an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

In some embodiments, the benzamide-based HDAC inhibitor comprises any one of chidamide, CXD101, entinostat, mocetinostat, and combinations thereof. In some embodiments, the benzamide-based HDAC inhibitor comprises chidamide.

In some embodiments, the method further comprises contacting the cell with a second agent (e.g., one or more additional latency reactivation agents). In some embodiments, the second agent comprises a topoisomerase inhibitor (e.g., topoisomerase II inhibitor), or a pharmaceutically acceptable prodrug, a pharmaceutically active metabolite, or a pharmaceutically acceptable salt thereof. In some embodiments, the topoisomerase inhibitor comprises any one of epirubicin, doxorubicin, mitoxantrone, amonafide, teniposide, and combinations thereof. In some embodiments, the topoisomerase inhibitor comprises epirubicin.

In some embodiments, the second agent comprises an Mdm2 inhibitor, or a pharmaceutically acceptable prodrug, a pharmaceutically active metabolite, or a pharmaceutically acceptable salt thereof. In some embodiments, the Mdm2 inhibitor comprises nutlin-3a, HDM201, or a combination thereof.

Chemical structures of the representative inhibitors are provided as follows:

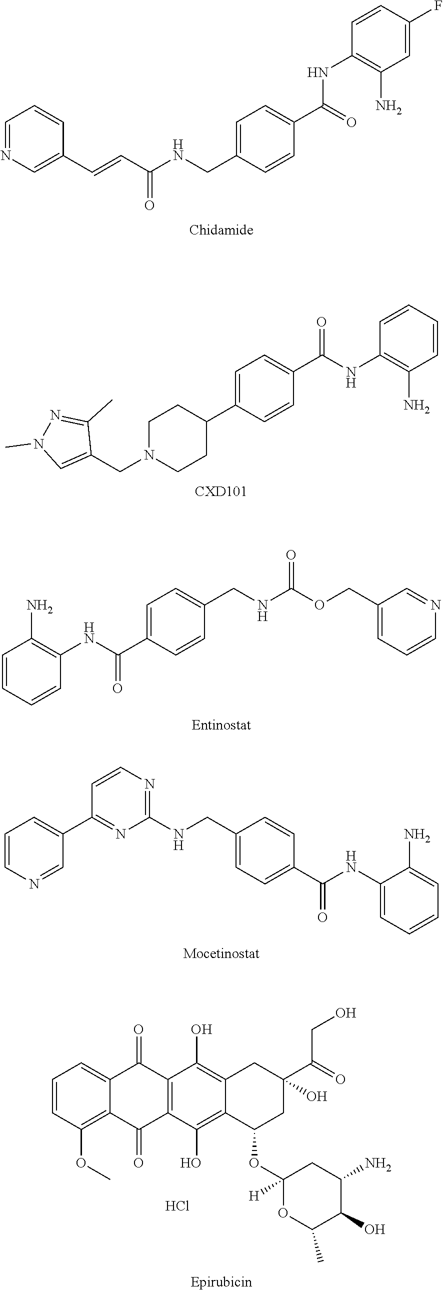 [SMILES: Nc1cc(F)ccc1NC(=O)c1ccc(CNC(=O)/C=C/c2cccnc2)cc1 ; Cc1nn(C)cc1CN1CCC(c2ccc(C(=O)Nc3ccccc3N)cc2)CC1 ; Nc1ccccc1NC(=O)c1ccc(CNC(=O)OCc2cccnc2)cc1 ; Nc1ccccc1NC(=O)c1ccc(CNc2nccc(-c3cccnc3)n2)cc1 ; COc1cccc2c1C(=O)c1c(O)c3c(c(O)c1C2=O)C[C@@](O)(C(=O)CO)C[C@@H]3O[C@H]1C[C@H](N)[C@@H](O)[C@H](C)O1 ; Cl]

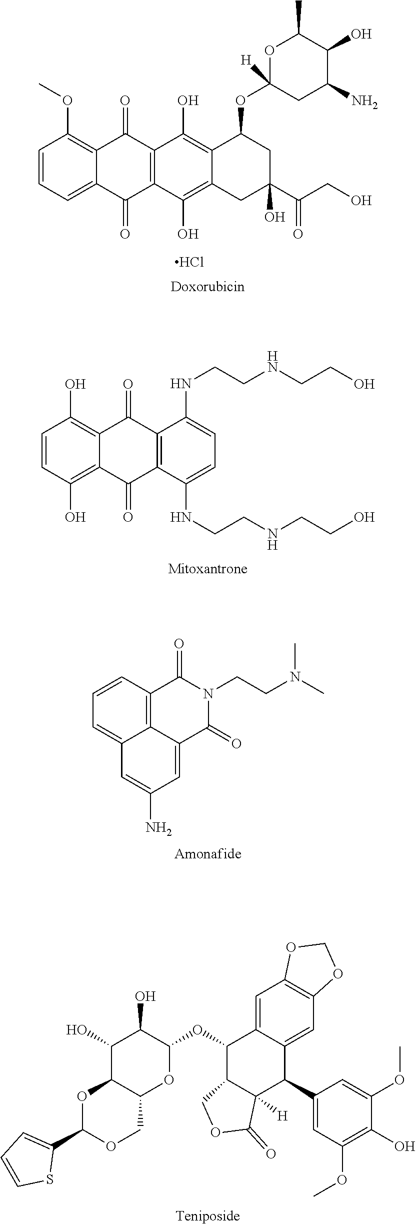 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1 ; Cl ; O=C1c2c(O)ccc(O)c2C(=O)c2c(NCCNCCO)ccc(NCCNCCO)c21 ; CN(C)CCN1C(=O)c2cccc3cc(N)cc(c23)C1=O ; COc1cc([C@@H]2c3cc4c(cc3[C@@H](O[C@@H]3O[C@@H]5CO[C@@H](c6cccs6)O[C@H]5[C@H](O)[C@H]3O)[C@H]3COC(=O)[C@H]23)OCO4)cc(OC)c1O]

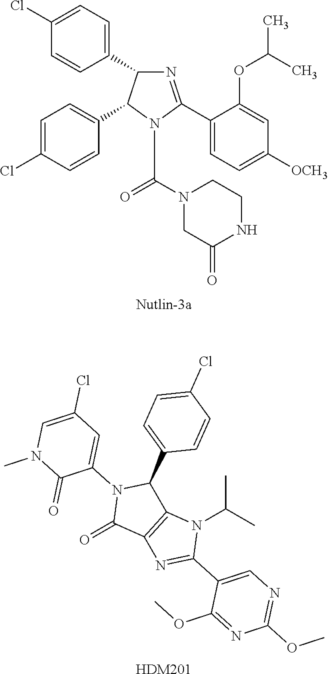

### b. Methods of Treating EBV-Associated Cancer

In another aspect, this disclosure provides a method of treating a subject having cancer associated with EBV infection. The method comprises administering to the subject an effective amount of a benzamide-based HDAC inhibitor, or a pharmaceutically acceptable prodrug, a pharmaceutically active metabolite, or a pharmaceutically acceptable salt thereof, wherein the benzamide-based HDAC inhibitor increases a level of expression or activity of an EBV-associated protein (e.g., Zta) in an EBV-positive cancer cell.

In some embodiments, the cell is an EBV-positive cancer cell. In some embodiments, the EBV-positive cancer cell is an EBV-positive gastric cancer cell. Examples of cancer associated with EBV infection may include, without limitation, nasopharyngeal carcinoma, gastric carcinoma, non-Hodgkin's lymphoma (anaplastic large cell lymphoma, angioimmunoblastic T-cell lymphoma, hepatosplenic T-cell lymphoma, B-cell lymphoma, Burkitt's lymphoma, reticuloendothelial proliferation, reticulocytosis, microglioma, diffuse large B-cell lymphoma, extranodal T/NK lymphoma/angiocentric lymphoma, follicular lymphoma, immunoblastic lymphoma, mucosa-associated lymphoid tissue lymphoma, B-cell chronic lymphocytic leukemia, mantle cell lymphoma, mediastinal large B-cell lymphoma, lymphoplasmacytic lymphoma, lymph node marginal zone B-cell lymphoma, splenic marginal zone lymphoma, intravascular large B-cell lymphoma, primary effusion lymphoma, lymphoma-like granulomatous, lymphomatoid granulomatosis, lymphomatosis, lympho, Angioimmunoblastic lymphadenopathy), leiomyosarcoma, concomitant lymphoproliferative disease, post-transplant lymphoproliferative disease, Hodgkin's lymphoma, and breast cancer.

In some embodiments, the benzamide-based HDAC inhibitor comprises any one of chidamide, CXD101, entinostat, mocetinostat, and combinations thereof. In some embodiments, the benzamide-based HDAC inhibitor comprises chidamide.

In some embodiments, the method further comprises administering to the subject a second agent. In some embodiments, the second agent comprises a topoisomerase inhibitor (e.g., topoisomerase II inhibitor), or a pharmaceutically acceptable prodrug, a pharmaceutically active metabolite, or a pharmaceutically acceptable salt thereof. In some embodiments, the topoisomerase inhibitor comprises any one of epirubicin, doxorubicin, mitoxantrone, amonafide, teniposide, and combinations thereof. In some embodiments, the topoisomerase inhibitor comprises epirubicin.

In some embodiments, the second agent comprises an Mdm2 inhibitor, or a pharmaceutically acceptable prodrug, a pharmaceutically active metabolite, or a pharmaceutically acceptable salt thereof. In some embodiments, the Mdm2 inhibitor comprises nutlin-3a, HDM201, or a combination thereof.

In some embodiments, the second agent comprises an anti-cancer agent (e.g., an immune checkpoint inhibitor).

Also within the scope of this disclosure is a method of eliciting or enhancing an immune response against an EBV-positive cancer cell in a subject infected with the EBV. The method comprises administering to the subject an effective amount of a benzamide-based HDAC inhibitor, or a pharmaceutically acceptable prodrug, a pharmaceutically active metabolite, or a pharmaceutically acceptable salt thereof, wherein the benzamide-based HDAC inhibitor increases a level of expression or activity of an EBV-associated protein (e.g., Zta) in the EBV-positive cancer cell.

The term “eliciting” or “enhancing” in the context of an immune response refers to triggering or increasing an immune response, such as an increase in the ability of immune cells to target and/or kill cancer cells or to target and/or kill pathogens and pathogen-infected cells (e.g., EBV-positive cancer cells).

The term “immune response,” as used herein, refers to any type of immune response, including, but not limited to, innate immune responses (e.g., activation of Toll receptor signaling cascade), cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

In yet another aspect, this disclosure provides a method of killing an EBV-positive cancer cell in a subject infected with the EBV. The method comprises administering to the subject an effective amount of a benzamide-based HDAC inhibitor, or a pharmaceutically acceptable prodrug, a pharmaceutically active metabolite, or a pharmaceutically acceptable salt thereof, wherein the benzamide-based HDAC inhibitor increases a level of expression or activity of an EBV-associated protein (e.g, Zta) in the EBV-positive cancer cell.

As used herein, the term “administering” refers to the delivery of cells by any route including, without limitation, oral, intranasal, intraocular, intravenous, intraosseous, intraperitoneal, intraspinal, intramuscular, intra-articular, intraventricular, intracranial, intralesional, intratracheal, intrathecal, subcutaneous, intradermal, transdermal, or transmucosal administration.

In some embodiments, the benzamide-based HDAC inhibitor is administered orally, topically, intravenously, intraperitoneally, intramuscularly, intralesionally, intrathecally, intranasally, subcutaneously, parenterally, transmucosally, sublingually, in controlled release, in delayed release, or as a suppository.

As used herein, “treatment” or “treating,” or “palliating” or “ameliorating” are used interchangeably. These terms refer to an approach for obtaining beneficial or desired results, including, but not limited to, a therapeutic benefit and/or a prophylactic benefit. By therapeutic benefit is meant any therapeutically relevant improvement in or effect on one or more diseases (e.g., cancer), conditions, or symptoms under treatment. For prophylactic benefit, the agent or the compositions thereof may be administered to a subject at risk of developing a particular disease, condition, or symptom, or to a subject reporting one or more of the physiological symptoms of a disease, even though the disease, condition, or symptom may not have yet been manifested.

In some embodiments, the method further comprises administering to the subject a second agent. In some embodiments, the second agent comprises a topoisomerase inhibitor (e.g., topoisomerase II inhibitor). In some embodiments, the topoisomerase inhibitor comprises any one of epirubicin, doxorubicin, mitoxantrone, amonafide, teniposide, and combinations thereof. In some embodiments, the topoisomerase inhibitor comprises epirubicin. In some embodiments, the second agent comprises an Mdm2 inhibitor. In some embodiments, the Mdm2 inhibitor comprises nutlin-3a, HDM201, or a combination thereof.

In some embodiments, the second agent is administered to the subject before, after, or concurrently with the benzamide-based HDAC inhibitor.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on the administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, the term “co-administration” or “co-administered” refers to the administration of at least two agent(s) or therapies to a subject. In some embodiments, in the treatment of tumors, particularly malignant tumors, the agents can be used alone or in combination with, e.g., chemotherapeutic, radiotherapeutic, apoptotic, anti-angiogenic agents and/or immunotoxins or coaguligands. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

In some embodiments, the method further comprises administering to the subject one or more additional therapeutic agents, such as antitumor/anticancer agents, including chemotherapeutic agents and immunotherapeutic agents.

A “chemotherapeutic agent” is a chemical compound useful in the treatment of cancer. Examples of chemotherapeutic agents include alkylating agents such as thiotepa and cyclophosphamide (CYTOXANTM); alkyl sulfonates such as busulfan, improsulfan and piposulfan; aziridines such as benzodopa, carboquone, methyldopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, see, e.g., Agnew Chem. Intl. Ed. Engl. 33:183-186 (1994); dynemicin, including dynemicin A; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERER, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, xeloda, gemcitabine, KRAS mutation covalent inhibitors and goserelin; and pharmaceutically acceptable salts, acids or derivatives of any of the above. Additional examples include irinotecan, oxaliplatinum, and other standard colon cancer regimens.

An “immunotherapeutic agent” may include a biological agent useful in the treatment of cancer. In some embodiments, the immunotherapeutic agent may include an immune checkpoint inhibitor (e.g., an inhibitor of PD-1, PD-L1, TIM-3, LAG-3, VISTA, DKG-α, B7-H3, B7-H4, TIGIT, CTLA-4, BTLA, CD160, TIM1, IDO, LAIR1, IL-12, or combinations thereof). Examples of immunotherapeutic agents include atezolizumab, avelumab, blinatumomab, daratumumab, cemiplimab, durvalumab, elotuzumab, laherparepvec, ipilimumab, nivolumab, obinutuzumab, ofatumumab, pembrolizumab, cetuximab, and talimogene.

In some embodiments, the method further comprises administering to the subject a cell (e.g., lymphocyte) transduced (e.g., virally transduced) with a recombinant T cell receptor (TCR). As used herein, a “recombinant TCR” refers to a TCR expressed from a polynucleotide that is introduced into the cell and not encoded by a chromosomal sequence in the cell before being introduced into the cell.

In some embodiments, the cell can be transiently or stably transduced by an expression vector (e.g., viral particle) harboring a polynucleotide encoding a TCR. In some embodiments, the recombinant TCR comprises a Zta-specific TCR, e.g., a TCR capable of recognizing transcriptional factor Zta or a fragment/variant thereof. In some embodiments, the recombinant TCR binds specifically to the transcriptional factor Zta or a fragment thereof. In some embodiments, the recombinant TCR comprises an amino acid sequence having at least 80% (e.g., 80%, 85%, 90%, 95%, 96%, 97%, 98%, 99%) sequence identity to an amino acid sequence of SEQ ID NOs: 37-48 or comprises an amino acid sequence of SEQ ID NOs: 37-48. In some embodiments, the recombinant TCR binds specifically to an antigen comprising an amino acid sequence of SEQ ID NOs: 49-53 and 55.

The cell may include CD4⁺T cells, CD8⁺T cells, natural killer T cells, γδ cells, and their precursor cells. For example, the CD8⁺T cells may be derived from any origin. The origin includes, without limitation, a human patient, who may or may not be the recipient of T cells.

Expression vectors for transducing the TCR can be any suitable expression vector. In embodiments, the expression vector comprises a modified viral polynucleotide, such as from an adenovirus, a herpesvirus, or a retrovirus, e.g., a lentiviral vector. The expression vector is not limited to recombinant viruses and includes non-viral vectors such as DNA plasmids and in vitro transcribed mRNA. In some embodiments, the cell can be cultured or expanded. The terms “culture,” “culturing,” “grow,” “growing,” “maintain,” “maintaining,” “expand,” “expanding,” etc., when referring to cell culture itself or the process of culturing, can be used interchangeably to mean that a cell (e.g., primary cell) is maintained outside its normal environment under controlled conditions, e.g., under conditions suitable for survival. Cultured cells are allowed to survive, and culturing can result in cell growth, stasis, differentiation or division. The term does not imply that all cells in the culture survive, grow, or divide, as some may naturally die or senesce. Cells are typically cultured in media, which can be changed during the course of the culture.

In some embodiments, the method further comprises administering to the subject an EBV vaccine and optionally an adjuvant. Examples of EVB vaccines include, without limitation, those described in the following patents or patent applications U.S. Pat. No. 10,300,129, WO2019103993, U.S. Pat. Nos 10,744,199, US20030152582, and U.S. Pat. No. 7,005,131, the disclosures of which are incorporated by reference in their entirety.

In some embodiments, the adjuvant may include aluminum hydroxide, lipid A, killed bacteria, polysaccharide, mineral oil, Freund's incomplete adjuvant, Freund's complete adjuvant, aluminum phosphate, iron, zinc, a calcium salt, acylated tyrosine, an acylated sugar, a cationically derivatized polysaccharide, an anionically derivatized polysaccharide, a polyphosphazene, a biodegradable microsphere, a monophosphoryl lipid A, and quil A.

### c. Zta-Specific TCR

In yet another aspect, this disclosure provides a TCR or antigen-binding fragment thereof comprising an amino acid sequence having at least 80% (e.g., 80%, 85%, 90%, 95%, 96%, 97%, 98%, 99%) sequence identity to an amino acid sequence of SEQ ID NOs: 37-48 or comprising an amino acid sequence of SEQ ID NOs: 37-48.

In some embodiments, the TCR or antigen-binding fragment thereof binds specifically to the transcriptional factor Zta or a fragment thereof. In some embodiments, the recombinant TCR binds specifically to an antigen comprising an amino acid sequence of SEQ ID NOs: 49-53 and 55.

As used herein, the term “T cell receptor” or “TCR” refers to a surface protein of a T cell that allows the T cell to recognize an antigen and/or an epitope thereof, typically bound to one or more major histocompatibility complex (MHC) molecules. A TCR functions to recognize an antigenic determinant and to initiate an immune response. Typically, TCRs are heterodimers comprising two different protein chains. In the vast majority of T cells, the TCR comprises an α chain and a β chain. Approximately 5% of T cells have TCRs made up of γ/δ chains. TCRs are membrane-anchored heterodimers that are found as part of a complex with a CD3 chain molecule. Each chain comprises two extracellular domains: a variable (V) region and a constant (C) region, the latter of which is membrane-proximal. The variable domains of α chains and β chains consist of three hypervariable regions that are also referred to as the complementarity determining regions (CDRs). The CDRs, in particular CDR3, are primarily responsible for contacting antigens and thus define the specificity of the TCR, although CDR1 of the α chain can interact with the N-terminal part of the antigen. CDR1 of the β chain interacts with the C-terminal part of the peptide. TCRs are also characterized by a series of highly conserved disulfide bonds that link the two chains.

In some embodiments, the TCR α chains may further comprise a TCR a transmembrane domain and/or a TCR a intracellular domain. Similarly, the TCR β chains may further comprise a TCR B transmembrane domain and/or a TCR β intracellular domain. The TCRs may further comprise a constant region derived from any suitable species, such as any mammal, e.g., human, rat, monkey, rabbit, donkey, or mouse. In some embodiments, the TCRs further comprise a human constant region. In some embodiments, the TCR constant region may be modified, for example, by the introduction of heterologous sequences, which may increase TCR expression and stability. In some embodiments, the TCR sequences are murinized or humanized.

In some embodiments, the TCR is an αβ heterodimeric TCR. In some embodiments, the TCR is an αβ single chain TCR (scTCR) or a TCR-like polypeptide. In some embodiments, the TCR as disclosed herein may be provided as a scTCR. A scTCR may comprise in one polypeptide chain a full or partial α chain sequence and a full or partial β chain sequence, which may be connected via a peptide linker. A scTCR can comprise a polypeptide of a variable region of a first TCR chain (e.g., an α chain) and a polypeptide of an entire (full-length) second TCR chain (e.g., a β chain), or vice versa. Furthermore, the scTCR can optionally comprise one or more linkers that join the two or more polypeptides together. The linker can be, for example, a peptide, which joins together two single chains, as described herein. As used herein, the phrase “TCR-like polypeptide” refers to a polypeptide that behaves similarly to a TCR in that it specifically binds to an MHC-bound peptide, optionally an MHC-bound phosphopeptide. A “TCR-like antibody” refers to an antibody, optionally a monoclonal antibody, which specifically recognizes an MHC-bound phosphopeptide of the presently disclosed subject matter. In some embodiments, such polypeptides are members of the Ig superfamily. In some embodiments, a TCR-like polypeptide is a single chain TCR (see, e.g., U.S. Patent Application Publication No. 2012/0252742; PCT International Patent Application Publication Nos. WO 1996/013593, WO 1999/018129, and WO 2004/056845; U.S. Pat. No. 7,569,664).

As used herein, a “fragment” or “portion” of a TCR or TCR-like polypeptide is a subsequence of a TCR or TCR-like polypeptide that retains a desired function of the TCR or TCR-like polypeptide. In some embodiments, a fragment or portion of a TCR or TCR-like polypeptide comprises the domain of the TCR or TCR-like polypeptide that binds to a phosphopeptide/MHC complex (e.g., a phosphopeptide/HLA-A2 complex). Thus, in some embodiments, the phrase “TCR, TCR-like molecule, or portion thereof” refers to TCRs, TCR-like molecules, and portions thereof that bind to phosphopeptide/MHC complexes, including but not limited to phosphopeptide/HLA-A2 complexes.

As used herein, the phrase “specific binding” refers to binding between a TCR, TCR-like molecule, or antigen-binding fragment thereof and an antigen and/or an epitope thereof (including but not limited to a peptide, optionally in complex with an MHC molecule) that is indicative of the presence of the antigen and/or the epitope thereof. As such, a TCR, TCR-like molecule, or antigen-binding fragment thereof is said to “specifically” bind an antigen and/or an epitope thereof when the dissociation constant (Kd) is less than about 1 μM, less than about 100 nM, or less than about 10 nM. Interactions between a TCR, TCR-like molecule, or antigen-binding fragment thereof and an epitope can also be characterized by an affinity constant (Kₐ). In some embodiments, a Kₐ of less than about 10⁷/M is considered “high affinity.”

In another aspect, the disclosure provides nucleic acids encoding a TCR or antigen-binding fragment thereof. In some embodiments, the TCR or antigen-binding fragment thereof is encoded by a single nucleic acid. In other embodiments, for example, in the case of a heterodimeric molecule or a polypeptide composed of more than one polypeptide chain. In some embodiments, the TCR or antigen-binding fragment thereof can be encoded by a plurality (e.g., two, three, four or more) nucleic acids.

In some embodiments, a single nucleic acid can encode a TCR or antigen-binding fragment thereof that comprises a single polypeptide chain, a TCR or antigen-binding fragment thereof that comprises two or more polypeptide chains, or a TCR or antigen-binding fragment thereof that comprises more than two polypeptide chains. For example, a single nucleic acid can encode two polypeptide chains of a TCR or antigen-binding fragment thereof comprising three, four or more polypeptide chains, or three polypeptide chains of a TCR or antigen-binding fragment thereof comprising four or more polypeptide chains. For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements and separated by internal ribosome entry site (IRES) sequences allowing for translation into separate polypeptides.

In some embodiments, a TCR or antigen-binding fragment thereof comprising two or more polypeptide chains is encoded by two or more nucleic acids. The number of nucleic acids encoding a TCR or antigen-binding fragment thereof can be equal to or less than the number of polypeptide chains in the TCR or antigen-binding fragment thereof (for example, when two or more polypeptide chains are encoded by a single nucleic acid).

In some embodiments, the nucleic acids of the disclosure can be DNA or RNA (e.g., mRNA).

In another aspect, the disclosure provides vectors comprising the nucleic acids encoding the TCRs or antigen-binding fragment thereof or the polypeptides as described above. The nucleic acids may be present in a single vector or separate vectors that are present in the same host cell or separate host cell.

In some embodiments, vectors can be derived from retroviruses, including avian reticuloendotheliosis virus (duck infectious anaemia virus, spleen necrosis virus, Twiehaus-strain reticuloendotheliosis virus, C-type retrovirus, reticuloendotheliosis virus Hungary-2 (REV-H-2)), and feline leukemia virus (FeLV)). Retroviral genomes have been modified for use as a vector (Cone & Mulligan, Proc. Natl. Acad. Sci., USA, 81:6349-6353, (1984)). Non-limiting examples of retroviruses include lentiviruses, such as human immunodeficiency viruses (HIV-1 and HIV-2), feline immunodeficiency virus (FIV), simian immunodeficiency virus (SIV), Maedi/Visna virus, caprine arthritis/encephalitis virus, equine infectious anaemia virus (EIAV), and bovine immunodeficiency virus (BIV); avian type C retroviruses, such as the avian leukosis virus (ALV); HTLV-BLV retroviruses, such as bovine leukaemia virus (BLV), human T cell lymphotropic virus (HTLV), and simian T cell lymphotropic virus; mammalian type B retroviruses, such as the mouse mammary tumor virus (MMTV); mammalian type C retroviruses, such as the murine leukaemia virus (MLV), feline sarcoma virus (FeSV), murine sarcoma virus, Gibbon ape leukemia virus, guinea pig type C virus, porcine type C virus, wooly monkey sarcoma virus, and viper retrovirus; spumavirus (foamy virus group), such as human spumavirus (HSRV), feline synctium-forming virus (FeSFV), human foamy virus, simian foamy virus, and bovine syncytial virus; and type D retroviruses, such as Mason-Pfizer monkey virus (MPMV), squirrel monkey retrovirus, and langur monkey virus.

In some embodiments, the vector comprises a retroviral vector or a lentiviral vector. In some embodiments, lentiviral and retroviral vectors may be packaged using their native envelope proteins or may be modified to be encapsulated with heterologous envelope proteins. Examples of envelope proteins include, but are not limited to, an amphotropic envelope, an ecotropic envelope, or a xenotropic envelope, or may be an envelope including amphotropic and ecotropic portions. The protein also may be that of any of the above-mentioned retroviruses and lentiviruses. Alternatively, the env proteins may be modified, synthetic or chimeric env constructs, or may be obtained from non-retro viruses, such as vesicular stomatitis virus and HVJ virus. Specific non-limiting examples include the envelope of Moloney Murine Leukemia Virus (MMLV), Rous Sarcoma Virus, Baculovirus, Jaagsiekte Sheep Retrovirus (JSRV) envelope protein, and the feline endogenous virus RD114; gibbon ape leukemia virus (GALV) envelope; baboon endogenous virus (BaEV) envelope; simian sarcoma-associated virus (SSAV) envelope; amphotropic murine leukemia virus (MLV-A) envelope; human immunodeficiency virus envelope; avian leukosis virus envelope; the endogenous xenotropic NZB viral envelopes; and envelopes of the paramyxoviridiae family such as, but not limited to, the HVJ virus envelope.

In another aspect, this disclosure further provides a cell (e.g., antigen-specific lymphocyte) comprising the nucleic acid or the vector, as described above. In some embodiments, the cell comprises an immune cell.

In some embodiments, the immune cell comprises a lymphocyte. In some embodiments, the lymphocyte comprises a T cell or a natural killer (NK) cell. In some embodiments, the T cell comprises a CD8+ T cell or a CD4+ T cell. In some embodiments, the T cell comprises a human T cell.

Lymphocytes are one subtype of white blood cells in the immune system. In some embodiments, lymphocytes may include tumor-infiltrating immune cells. Tumor-infiltrating immune cells consist of both mononuclear and polymorphonuclear immune cells (i.e., T cells, B cells, natural killer cells, macrophages, neutrophils, dendritic cells, mast cells, eosinophils, basophils, etc.) in variable proportions. In some embodiments, lymphocytes may include tumor-infiltrating lymphocytes (TILs). TILs are white blood cells that have left the bloodstream and migrated towards a tumor. TILs can often be found in the tumor stroma and within the tumor itself. In some embodiments, TILs are “young” T cells or minimally cultured T cells. In some embodiments, the young cells have a reduced culturing time (e.g., between about 22 to about 32 days in total). In some embodiments, the lymphocytes express CD27.

In some embodiments, lymphocytes may include peripheral blood lymphocytes (PBLs). In some embodiments, lymphocytes include T lymphocytes (T cells) and/or natural killer cells (NK cells).

In some embodiments, the lymphocytes may be autologous, allogeneic, syngeneic, or xenogeneic with respect to the subject. In some embodiments, the lymphocytes are autologous in order to reduce an immunoreactive response against the lymphocyte when reintroduced into the subject for immunotherapy treatment.

In some embodiments, the T cells are CD8+ T cells. In some embodiments, the T cells are CD4+ cells. In some embodiments, the NK cells are CD 16+ CD56+ and/or CD57+ NK cells. NKs are characterized by their ability to bind to and kill cells that fail to express self MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

In another aspect, the above-described TCRs, polypeptides, nucleic acids, vectors, or cells can be incorporated into compositions, e.g., pharmaceutical compositions suitable for administration, or kits.

In some embodiments, the pharmaceutical compositions may include a population of lymphocytes described herein (e.g., lymphoctyes transduced with a Zta-specific TCR or antigen-binding fragment thereof) and a pharmaceutically acceptable carrier and/or excipient. In some embodiments, the pharmaceutical compositions may comprise substantially isolated/purified lymphocytes and a pharmaceutically acceptable carrier in a form suitable for administration to a subject. Pharmaceutically acceptable carriers are determined in part by the particular composition being administered, as well as by the particular method used to administer the composition. The pharmaceutical compositions are generally formulated in full compliance with all Good Manufacturing Practice (GMP) regulations of the U.S. Food and Drug Administration.

### B. COMPOSITIONS AND KITS

In another aspect, this disclosure also provides a composition, e.g., pharmaceutical composition, for eliciting or enhancing an immune response against an EBV-positive cancer cell in a subject infected with the EBV. In some embodiments, the composition comprises: (i) benzamide-based HDAC inhibitor; (ii) a topoisomerase inhibitor (e.g., topoisomerase II inhibitor) or an Mdm2 inhibitor; and (iii) optionally a pharmaceutically acceptable carrier.

In some embodiments, the benzamide-based HDAC inhibitor comprises any one of chidamide, CXD101, entinostat, mocetinostat, and combinations thereof. In some embodiments, the topoisomerase inhibitor comprises any one of epirubicin, doxorubicin, mitoxantrone, amonafide, teniposide, and combinations thereof. In some embodiments, the Mdm2 inhibitor comprises nutlin-3a, HDM201, or a combination thereof. In some embodiments, the composition comprises chidamide, epirubicin, and optionally the pharmaceutically acceptable carrier.

In some embodiments, the composition is an immunogenic composition (e.g., vaccine) optionally comprising a pharmaceutically acceptable diluent, vehicle, one or more immunological adjuvants, or combinations thereof.

Pharmaceutical compositions may be formulated in a conventional manner using one or more physiologically acceptable carriers or excipients. Thus, the HDAC, topoisomerase, or Mdm2 inhibitors, or their analogs/derivatives, or their physiologically acceptable salts and solvates may be formulated for administration by, for example, injection, inhalation or insufflation (either through the mouth or the nose) or oral, buccal, parenteral or rectal administration. In one embodiment, the agent is administered locally, e.g., at the site where the target cells are present, such as by the use of a patch.

Pharmaceutical compositions can be formulated for a variety of loads of administration, including systemic and topical or localized administration. Techniques and formulations generally may be found in Remmington's Pharmaceutical Sciences, Meade Publishing Co., Easton, PA. For systemic administration, injection is preferred, including intramuscular, intravenous, intraperitoneal, and subcutaneous. For injection, the agents can be formulated in liquid solutions, preferably in physiologically compatible buffers such as Hank's solution or Ringer's solution. In addition, the agents may be formulated in solid form and redissolved or suspended immediately prior to use. Lyophilized forms are also included.

For oral administration, the pharmaceutical compositions may take the form of, for example, tablets, lozenges, or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicles before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoate or sorbic acid). The preparations may also contain buffer salts, flavoring, coloring, and sweetening agents as appropriate. Preparations for oral administration may be suitably formulated to give controlled release of the active compound.

Pharmaceutical compositions that may oxidize and lose biological activity, especially in a liquid or semisolid form, may be prepared in a nitrogen atmosphere or sealed in a type of capsule and/or foil package that excludes oxygen (e.g., Capsugel™).

For administration by inhalation, the agents may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, e.g., gelatin, for use in an inhaler or insufflator may be formulated containing a powder mix of the agent and a suitable powder base such as lactose or starch.

Pharmaceutical compositions may be formulated for parenteral administration by injection, e.g., by bolus injection or continuous infusion. Formulations for injection may be presented in unit dosage form, e.g., in ampoules or in multi-dose containers, with an added preservative. The agents may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. The agents may also be formulated in rectal compositions such as suppositories or retention enemas, e.g., containing conventional suppository bases such as cocoa butter or other glycerides.

In addition to the formulations described previously, pharmaceutical compositions may also be formulated as a depot preparation. Such long-acting formulations may be administered by implantation (for example, subcutaneously or intramuscularly) or by intramuscular injection. Thus, for example, the agents may be formulated with suitable polymeric or hydrophobic materials (for example, as an emulsion in an acceptable oil) or ion exchange resins, or as sparingly soluble derivatives, for example, as a sparingly soluble salt. Controlled release formula also includes patches, e.g., transdermal patches. Patches may be used with a sonic applicator that deploys ultrasound in a unique combination of waveforms to introduce drug molecules through the skin that normally could not be effectively delivered transdermally.

Pharmaceutical compositions (including cosmetic preparations) may comprise from about 0.00001 to 100%, such as from 0.001 to 10% or from 0.1% to 5% by weight of one or more agents described herein.

A pharmaceutical composition described herein can also be incorporated into a topical formulation containing a topical earner that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier does not adversely affect the active agent or other components of the topical formulation. Examples of suitable topical carriers for use herein include water, alcohol, and other nontoxic organic solvents, glycerin, mineral oil, silicone, petroleum jelly, lanolin, fatty acids, vegetable oils, parabens, waxes, and the like.

Formulations may be colorless, odorless ointments, lotions, creams, microemulsions, and gels. Pharmaceutical compositions may be incorporated into ointments, which generally are semisolid preparations which are typically based on petrolatum or other petroleum derivatives. The specific ointment base to be used, as will be appreciated by those skilled in the art, is one that will provide for optimum drug delivery and, preferably, will provide for other desired characteristics as well, e.g., emolliency or the like. As with other carriers or vehicles, an ointment base should be inert, stable, nonirritating, and nonsensitizing. As explained in Remington's, ointment bases may be grouped into four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxy stearic sulfate, anhydrous lanolin, and hydrophilic petrolatum. Emulsion ointment bases are either water-in-oil (W/O) emulsions or oil-in-water (O/W) emulsions and include, for example, cetyl alcohol, glyceryl monostearate, lanolin, and stearic acid. Exemplary water-soluble ointment bases are prepared from polyethylene glycols (PEGs) of varying molecular weight; again, reference may be had to Remington's, supra, for further information.

Pharmaceutical compositions may be incorporated into lotions, which generally are preparations to be applied to the skin surface without friction and are typically liquid or semiliquid preparations in which solid particles, including the active agent, are present in a water or alcohol base. Lotions are usually suspensions of solids and may comprise a liquid oily emulsion of the oil-in-water type. Lotions are preferred formulations for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like. An exemplary lotion formulation for use in conjunction with the present method contains propylene glycol mixed with hydrophilic petrolatum such as that which may be obtained under the trademark Aquaphor™ from Beiersdorf, Inc. (Norwalk, Conn.).

Pharmaceutical compositions may be incorporated into creams, which generally are viscous liquid or semisolid emulsions, either oil-in-water or water-in-oil. Cream bases are water-washable and contain an oil phase, an emulsifier, and an aqueous phase. The oil phase is generally comprised of petrolatum and a fatty alcohol such as cetyl or stearyl alcohol; the aqueous phase usually, although not necessarily, exceeds the oil phase in volume and generally contains a humectant. The emulsifier in a cream formulation, as explained in Remington's, supra, is generally a nonionic, anionic, cationic or amphoteric surfactant.

Pharmaceutical compositions may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase, and a water phase are necessary. Suitable surfactants include any surfactants that are useful in the preparation of emulsions, e.g., emulsifiers that are typically used in the preparation of creams. The co-surfactant (or “co-emulsifier”) is generally selected from the group of polyglycerol derivatives, glycerol derivatives, and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprylic and capric triglycerides and oleoyl macrogol glycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono-di- and triglycerides, mono- and di-esters of PEG (e.g., oleoyl macrogol glycerides), etc.

Pharmaceutical compositions may be incorporated into gel formulations, which generally are semisolid systems consisting of either suspension made up of small inorganic particles (two-phase systems) or large organic molecules distributed substantially uniformly throughout a carrier liquid (single-phase gels). Single-phase gels can be made, for example, by combining the active agent, a carrier liquid, and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methyl hydroxy cellulose, polyoxyethylene-polyoxypropylene, hydroxyethylcellulose, and gelatin. Although gels commonly employ aqueous carrier liquid, alcohols and oils can be used as the carrier liquid as well.

Various additives, known to those skilled in the art, may be included in formulations, e.g., topical formulations. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients, and preservatives. An optimum topical formulation comprises approximately: 2 wt. % to 60 wt. % solubilizer and/or skin permeation enhancer; 2 wt. % to 50 wt. % emulsifiers; 2 wt. % to 20 wt. % emollient; and 0.01 to 0.2 wt. % preservative, with the active agent and carrier (e.g., water) making of the remainder of the formulation. A skin permeation enhancer serves to facilitate passage of therapeutic levels of active agent to pass through a reasonably sized area of unbroken skin. Suitable enhancers are well known in the art and include, for example: lower alkanols such as methanol ethanol and 2-propanol; alkyl methyl sulfoxides such as dimethylsulfoxide (DMSO), decylmethylsulfoxide (C.sub.lO MSO) and tetradecyl methyl sulfoxide; pyrrolidones such as 2-pyrrolidone, N-methyl-2-pyrrolidone and N-(-hydroxyethyl) pyrrolidone; urea; N,N-diethyl-m-toluamide; C.sub.2-C. sub.6 alkane diols; miscellaneous solvents such as dimethylformamide (DMF), N,N-dimethylacetamide (DMA), and tetrahydrofurfuryl alcohol; and the 1-substituted azacycloheptan-2-ones, particularly 1-n-dodecylcyclazacycloheptan-2-one (laurocapram; available under the trademark Azone® from Whitby Research Incorporated, Richmond, Va.).

Examples of solubilizers include, but are not limited to, the following: hydrophilic ethers such as diethylene glycol monoethyl ether (ethoxydiglycol, available commercially as Transcutol™) and diethylene glycol monoethyl ether oleate (available commercially as Softcutol™); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol™); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol-2 (PPG-2) myristyl ether propionate, and the like.

Other active agents may also be included in formulations, e.g., anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxy dibenzoyl methane), p-aminobenzoic acid (PABA) and derivatives thereof, and salicylates (e.g., octyl salicylate). In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation. Topical skin treatment compositions can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507. Accordingly, also provided are closed containers containing a cosmetically acceptable composition.

In some embodiments, a pharmaceutical formulation is provided for oral or parenteral administration, in which case the formulation may comprise an activating compound-containing microemulsion as described above and may contain alternative pharmaceutically acceptable carriers, vehicles, additives, etc. particularly suited to oral or parenteral drug administration. Alternatively, an activating compound-containing microemulsion may be administered orally or parenterally substantially, as described above, without modification.

A composition described herein can be provided in a kit. In some embodiments, the kit comprises: (i) benzamide-based HDAC inhibitor; (ii) a topoisomerase inhibitor (e.g., topoisomerase II inhibitor) or an Mdm2 inhibitor; and (iii) optionally a pharmaceutically acceptable carrier.

In some embodiments, the benzamide-based HDAC inhibitor comprises any one of chidamide, CXD101, entinostat, mocetinostat, and combinations thereof. In some embodiments, the topoisomerase inhibitor comprises any one of epirubicin, doxorubicin, mitoxantrone, amonafide, teniposide, and combinations thereof. In some embodiments, the Mdm2 inhibitor comprises nutlin-3a, HDM201, or a combination thereof. In some embodiments, the kit comprises chidamide, epirubicin, and optionally the pharmaceutically acceptable carrier.

In some embodiments, the kit also includes an additional therapeutic agent (e.g., anti-cancer agent). For example, the kit includes a first container that contains the composition and a second container for the additional therapeutic agent.

The kit can include one or more containers for the composition or compositions containing an HDAC inhibitor, a topoisomerase inhibitor, an Mdm2 inhibitor, or combinations thereof. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. For example, the kit includes a plurality of syringes, ampules, foil packets, blister packs, or medical devices, e.g., each containing a single combination unit dose. The containers of the kits can be airtight, waterproof (e.g., impermeable to changes in moisture or evaporation), and/or light-tight.

The kit optionally includes a device suitable for administration of the composition, e.g., a syringe or other suitable delivery device. The device can be provided pre-loaded with one or both of the agents or can be empty, but suitable for loading.

In addition to the composition, the kit can include other ingredients, such as a solvent or buffer, a stabilizer, or a preservative. The composition can be provided in any form, e.g., liquid, dried or lyophilized form, preferably substantially pure and/or sterile. When the agents are provided in a liquid solution, the liquid solution preferably is an aqueous solution. When the agents are provided as a dried form, reconstitution generally is by the addition of a suitable solvent and acidulant. The acidulant and solvent, e.g., an aprotic solvent, sterile water, or a buffer, can optionally be provided in the kit.

In some embodiments, the kit may further include informational materials. The informational material of the kits is not limited in its form. In one embodiment, the informational material can include information about the production of the composition, concentration, date of expiration, batch or production site information, and so forth. In one embodiment, the informational material relates to methods of administering the composition, e.g., in a suitable dose, dosage form, or mode of administration (e.g., a dose, dosage form, or mode of administration described herein), to treat a subject in need thereof. In one embodiment, the instructions provide a dosing regimen, dosing schedule, and/or route of administration of the composition or the additional therapeutic agent. The information can be provided in a variety of formats, including printed text, computer-readable material, video recording, or audio recording, or information that contains a link or address to substantive material.

### C. DEFINITIONS

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, the term “recombinant” refers to a cell, microorganism, nucleic acid molecule or vector that has been modified by the introduction of an exogenous nucleic acid molecule or has controlled expression of an endogenous nucleic acid molecule or gene., Deregulated or altered to be constitutively altered, such alterations or modifications can be introduced by genetic engineering. Genetic alteration includes, for example, modification by introducing a nucleic acid molecule encoding one or more proteins or enzymes (which may include an expression control element such as a promoter), or addition, deletion, substitution of another nucleic acid molecule., Or other functional disruption of, or functional addition to, the genetic material of the cell. Exemplary modifications include modifications in the coding region of a heterologous or homologous polypeptide derived from the reference or parent molecule or a functional fragment thereof.

As used herein, the term “antigen” is a molecule and/or substance that can generate peptide fragments that are recognized by a TCR and/or induces an immune response. An antigen may contain one or more “epitopes.” In some embodiments, the antigen has several epitopes. An epitope is recognized by a TCR, an antibody or a lymphocyte in the context of an MHC molecule.

Also within the scope of this disclosure are the variants of the TCR or the polypeptide, as described above. As used herein, the term “variant” refers to a first molecule that is related to a second molecule (also termed a “parent” molecule). The variant molecule can be derived from, isolated from, based on or homologous to the parent molecule. A “functional variant” of a protein as used herein refers to a variant of such protein that retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide, or peptide. Functional variants may be naturally occurring or may be man-made.

In some embodiments, a variant of the TCR or the polypeptide may include one or more conservative modifications. The variant with one or more conservative modifications may retain the desired functional properties, which can be tested using the functional assays known in the art.

As used herein, the term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine) includes one or more conservative modifications. The variant of the TCR or the polypeptide with one or more conservative modifications may retain the desired functional properties, which can be tested using the functional assays known in the art.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program, using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum62 matrix or a PAM250 matrix, and a gap weight of 16, 14, 12, 10, 8, 6, or 4 and a length weight of 1, 2, 3, 4, 5, or 6.

The term “homolog” or “homologous,” when used in reference to a polypeptide, refers to a high degree of sequence identity between two polypeptides, or to a high degree of similarity between the three-dimensional structure or to a high degree of similarity between the active site and the mechanism of action. In some embodiments, a homolog has a greater than 60% sequence identity, and more preferably greater than 75% sequence identity, and still more preferably greater than 90% sequence identity, with a reference sequence. The term “substantial identity,” as applied to polypeptides, means that two peptide sequences, when optimally aligned, such as by the programs GAP or BESTFIT using default gap weights, share at least 75% sequence identity.

Also within the scope of this disclosure are the variants, mutants, and homologs with significant identity to the disclosed TCRs or polypeptides. For example, such variants and homologs may have sequences with at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the sequences of TCRs or polypeptides described herein. As used herein, the term “contacting,” when used in reference to any set of components, includes any process whereby the components to be contacted are mixed into the same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C. “Contacting” a target nucleic acid or a cell with one or more reaction components includes any or all of the following situations: (i) the target or cell is contacted with a first component of a reaction mixture to create a mixture; then other components of the reaction mixture are added in any order or combination to the mixture; and (ii) the reaction mixture is fully formed prior to mixture with the target or cell.

As used herein, a “subject” refers to a human and a non-human animal. Examples of a non-human animal include all vertebrates, e.g., mammals, such as non-human mammals, non-human primates (particularly higher primates), dog, rodent (e.g., mouse or rat), guinea pig, cat, and rabbit, and non-mammals, such as birds, amphibians, reptiles, etc. In one embodiment, the subject is a human. In another embodiment, the subject is an experimental animal or animal suitable as a disease model.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “cancer” refers to a disease characterized by rapid and uncontrolled growth of abnormal cells. Cancer cells can spread locally or through the bloodstream and lymphatic system to other body parts. The terms “tumor” and “cancer” are used interchangeably herein. For example, both terms encompass solid and liquid, such as diffuse or circulating tumors. As used herein, the term “cancer” or “tumor” includes premalignant as well as malignant cancers and tumors.

“Sample,” “test sample,” and “patient sample” may be used interchangeably herein. The sample can be a sample of, serum, urine plasma, amniotic fluid, cerebrospinal fluid, cells (e.g., antibody-producing cells) or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest such as antibodies. The sample may be any tissue sample from the subject. The sample may comprise protein from the subject.

The terms “inhibit” and “antagonize,” as used herein, mean to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down-regulate a protein, a gene, and an mRNA stability, expression, function, and activity, e.g., antagonists.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays. Doses are often expressed in relation to bodyweight. Thus, a dose which is expressed as [g, mg, or other unit]/kg (or g, mg etc.) usually refers to [g, mg, or other unit] “per kg (or g, mg etc.) bodyweight,” even if the term “bodyweight” is not explicitly mentioned.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. When used in this document, the term “exemplary” is intended to mean “by way of example” and is not intended to indicate that a particular exemplary item is preferred or required.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### D. EXAMPLES

### Example 1

This example describes the materials and methods used in the subsequent EXAMPLES below.

### Western Blotting

Cells were washed once with ice-cold phosphate-buffered saline (PBS) before being scraped off the dish in PBS. Cells were pelleted at 250×g for 5 min at 4° C. and lysed with 8M urea buffer on ice for 30 min. Lysates were cleared by centrifugation at 2500×g for 20 min at 4° C. Protein concentration was determined by the Bradford assay. Samples were boiled in 1×SDS sample buffer for 10 min and subsequently separated on SDS-acrylamide gels. Proteins were transferred on a nitrocellulose membrane (GE Healthcare), blocked with 5% dried skimmed milk in 0.1% Tween-20 in PBS (PBS-T) for 1 h, and incubated with the primary antibody diluted in 5% milk in PBS-T overnight at 4° C. After washing, membranes were incubated with the appropriate HRP-conjugated secondary antibody (1:2000; Dako) in PBS-T for 1 h at room temperature before visualizing with an enzymatic chemiluminescence detection system (GE Healthcare). Antibodies used in this study are listed in Table 1.

### Immunofluorescence

Cells were seeded on 13 mm coverslips or 96 well-plates 24 h prior to the treatment. After washing with PBS, cells were fixed by the addition of 4% formaldehyde (Life Technologies) in PBS for 10 min, washed three times with PBS, and incubated with 0.1% Triton X-100 for 20 min. After three more washes, coverslips were incubated with Signal Enhancer for 30 min. Following one wash, cells were incubated with the primary antibody diluted in antibody diluent (Dako) with a background reducing agent for 90 min. After washing three times, coverslips were incubated with a secondary antibody solution containing DAPI diluted in antibody diluent for 1 h in the dark. Coverslips were washed three times with PBS and once in purified water before coverslips were mounted on Polysine Microscope Adhesion slides with ProLong Gold Antifade Mountant (Life Technologies). Cells affixed on slides were visualized using a Zeiss LSM 710 laser scanning confocal microscope, while cells on 96 well plates were visualized using the Operetta High Content Screening System (PerkinElmer).

### High-Throughput Screening

Pharmakon 1600 library (MicroSource Discovery Systems) was used for the high-throughput screening. Experiments were run in duplicate. Liquid handling for cell plating, drug treatment, and cell fixation was performed using a JANUS PerkinElmer Automated Workstation (PerkinElmer). All washing steps of the staining procedure were performed with a BioTek Microplate Washer EL×405 Select CV. All other staining steps were performed with a FlexDrop PLUS (PerkinElmer). Cells were visualized using a GE IN Cell Analyzer 6000 (GE Healthcare Life Sciences) with a 10× objective. Percentages of Zta-positive cells within the wells were assessed with a protocol established within the Columbus software (PerkinElmer). Z-scores were calculated by the TDI Cellular High Throughput Screening Facility (University of Oxford). Compounds fulfilling the following criteria were considered as potential hits: Z-score≥1.96, Zta expression levels ≥5%, and wells containing more than 1000 cells after treatment.

### RNA Extraction and RT-qPCR

RNA was extracted from cells using the RNeasy Mini Kit (Qiagen), following the manufacturer's instructions. Possible contamination with genomic DNA was removed by treatment with DNase I according to the manufacturer's instructions. One μg was used to generate cDNA using the SuperScript Reverse Transcriptase III System following the manufacturer's instructions. QuantiTect SYBR Green PCR Kit was used to perform RT-qPCR using the StepOnePlus real-time PCR system (Applied Biosystems). Reactions were performed in duplicate and in a final volume of 20 μL. Relative mRNA abundances were normalized to endogenous glyceraldehyde 3-phosphate dehydrogenase (GAPDH) mRNA levels. Shown qPCR results were analyzed by the ΔΔCt-method, with the threshold cycles being determined by the Applied Biosystems software. Primers used in this study are listed in Table 2.

### RNA Interference

For TP53 silencing, siRNA oligos were purchased from Sigma-Aldrich. Cells were transiently transfected by reverse transfection using Lipofectamine RNAiMAX transfection reagent and 2 pmol/well (96 well plate) or 20 pmol/dish (6 mm dish) siRNAs for 48 h prior to the treatments. The efficiency of transfection was confirmed by immunofluorescence and Western blot. siRNAs used in this study are listed in Table 2.

cDNA Overexpression

For overexpression experiments, cells were transfected with 1 μg (60 mm dish) pcDNA3.1 empty vector (EV), pcDNA3.1-wtp53-HA, pcDNA3-SENP1-wt, or pcDNA3-SENP1-C603S vectors using Fugene transfection reagent following the manufacturer's instructions. After 24 h, the cells were treated with appropriate drugs in order to reactivate EBV.

### Chromatin Immunoprecipitation

Twenty million cells were cross-linked on the plate by adding 1% formaldehyde in the culture medium for 10 min at room temperature. The reaction was stopped by adding 125 mM glycine and incubating for 10 min at room temperature. Cells were washed twice with cold PBS and collected by scraping. Cells were lysed in SDS buffer (1% SDS, 10 mM EDTA, 50 mM Tris pH 8.1) for 30 min on ice. For chromatin shearing, cell lysates were sonicated using the Covaris S220 ultrasonicator (Covaris). One percent of chromatin was conserved as an input sample. Fragmented chromatin was diluted ten times with Chromatin Dilution Buffer (0.01% SDS, 1.1% TritonX-100, 1.2 mM EDTA, 16.7 mM Tris-HCl pH 8.1, 167 mM NaCl) and precleared with Protein A/G magnetic beads mix (ThermoFisher) for 1 h at +4° C. on a rotator. Precleared chromatin was incubated with 5 μg anti-p53 DO1 antibody or anti-mouse IgG overnight on a rotator at +4° C. The day after, samples were incubated with Protein A/G magnetic beads mix for 1 h at +4° C. on a rotator. The beads were collected using magnet and washed five times with cold RIPA washing buffer (50 mM Hepes-KOH PH 7.6, 500 mM LiCl, 1 mM EDTA, 1% NP-40, 0.7% Na-deoxycholate) and twice with TE/NaCl buffer (10 mM Tris pH 8.0, 1 mM EDTA, 50 mM NaCl). DNA was eluted by incubating the beads in Elution buffer (50 mM Tris-HCl, pH 8.0, 10 mM EDTA, 1% SDS) for 30 min at 65° C. Eluate was separated from the beads and reverse cross-linked at 65° C. overnight. The day after, samples were incubated with 0.5 μg RNase A at 37° C. for 30 min and then with 20 μg proteinase K at 55° C. for 30 min. DNA was purified using the Qiagen PCR Purification Kit (Qiagen). qPCR analysis of samples was performed using PowerUp SYBR green qPCR master mix (Applied Biosystems). qPCR was run on the StepOnePlus system. ChIP-qPCR primers used in this study are listed in Table 2.

qPCR for EBV Genome Load

Cells were washed once with ice-cold PBS before being scraped off the dish in PBS. Subsequently, the DNA was isolated using the QIAGEN DNeasy Blood & Tissue Kit following the manufacturer's instructions. The TaqMan Universal PCR Master Mix was used to perform qPCR using the StepOnePlus real-time PCR system (Applied Biosystems). DNA from Namalwa cells, containing two integrated EBV genomes per cell, was used as the standard to prepare a calibration curve. qPCR primers used in this study are listed in Table S1.

### Identification, Isolation, and Single-Cell Sorting of EBV Zta-Specific TCRs

Healthy donor PBMCs were screened for CD8⁺ T cell responses to EBV lytic stage antigen Zta using IFNγ ELISpot. Following this, PBMCs from donors whose T cells produced IFNγ in response to complete Zta peptide pool (47× 18 mers, overlapping by 10 amino acids) were then used for an IFNγ secretion assay (Human IFN-γ Secretion Detection Kit (PE), Miltenyi Biotech), performed to capture live antigen-specific T cells. PBMCs were stimulated for 6 hours at 37° C. with 2 μg/mL complete Zta peptide pool before following manufacturer's instructions and performing FACS staining for 20 minutes at room temperature (CD3-APC-H7, CD4-PE-Cy7, CD8-BV421 (all BD), Live/Dead stain AmCyan (Life Technologies)). Stained cells were single-sorted using the FACS Aria III cell sorter (BD) into 96-well PCR plates on ice before storing immediately in −80° C.

Single TCR Cloning, Transfection, and Transduction into T Cells

Paired TCR α and β genes from single-sorted Zta-specific T cells were amplified using a series of PCRs as described previously (Hamana, H., et al. (2016). Biochemical and Biophysical Research Communications 474, 709-714.). Paired α and β genes were sequenced and cloned into the pMX-IRES-GFP retroviral vector (Cell Biolabs) before transfecting into the retroviral packaging cell lines PlatGP and PG13 (ATCC). Viral supernatant from PG13 was collected and used to transduce the TCR-negative cell line SKW3 and activated primary T cells from a healthy donor who has previously tested negative for IFNγ responses to Zta peptide. Transduction was performed via centrifugation at 100 g for 2 h at 32° C. before returning cells to incubator for culture. Transduced SKW3 cells were analyzed 2 days later via flow cytometry using GFP expression, and the expression of TCR and CD3 on the surface using FACS (stained with CD3-APC-H7 and TCRαβ-APC, both BD). The transduced cells were cultured for approximately 10 days before GFP⁺ CD3⁺ TCR⁺ cells were sorted using the FACS Aria II, expanded, and used for further assays. Primary T cells were activated using CD3/CD28 Dynabeads (Thermofisher) at a 1:1 ratio for 3 days in the presence of IL-2 (100 U/mL). Transduction of the activated primary T cells was performed in the same way as the SKW3 cell transduction, except for the addition of IL-2 (50 U/mL) to each well prior to centrifugation with the virus supernatant. The next day after transduction, the R10 media was replaced and supplemented with fresh IL-2 (50 U/mL). The efficiency of transduction in the primary T cells was assessed 2-3 days after infection by analyzing the GFP expression. The cells were then expanded for a further 10 days, with the addition of 50 U/mL IL-2 every two to three days, before the CD3⁺ CD8⁺ GFP⁺ T cells were sorted. The sorted cells were either used immediately for functional studies or cultured for up to 7 days.

### Characterization of Zta-Specific TCRs

The functional capability of expressed TCRs was assessed by stimulating the T cells using responses to the complete Zta peptide pool. Peptide stimulation assays were performed by pulsing autologous EBV-transformed lymphoblastoid B-cell lines (BCLs) with the complete Zta peptide pool or an irrelevant peptide for 1 hr at 37° C. Wells pre-coated with 1 μg/mL anti-CD3 OKT3 antibody and 1 μg/mL anti-CD28 antibody (eBioscience) were also used as additional positive stimulation controls. This was followed by washing and co-culturing with the SKW3-TCR cells for 18 h. The cells were then washed and stained with CD3-APC-H7, TCRαβ-APC, and CD69-PE (all BD), before analyzing upregulation of CD69, compared to a BCL only or irrelevant peptide-pulsed control. The supernatant was collected from all peptide stimulation and killing assays and was used to analyze for secreted cytokines IL-2, IFNγ, and TNFα using the U-plex multiplex ELISA system (Meso Scale Discovery). The HLA restriction of each Zta-specific TCR was determined via peptide stimulation assays using partially histocompatible BCLs. The specific epitope of each TCR was determined via peptide stimulation assays using several smaller pools of 18 mer peptides, followed by stimulation assays with specific overlapping 11 mers and 9 mers and assessing CD69 upregulation.

### Evaluation of the Killing Ability of Zta-Specific TCR-T Cells

EBV+GC cells were treated with either 2.5 μM of CXD101, 5 μM of chidamide, or 5 μM of SAHA, and DMSO was used as a negative control. Approximately 10×10⁵ AGS-EBV cells were plated in 6 mm culture dishes the day before treatment to allow 50% confluence. The next day, the media was removed, and the cells were treated for 48 hours with the HDACi or DMSO. After 48 hours, the cells were dissociated with trypsin-EDTA and counted. The dissociated cells were plated in the wells of a flat-bottomed 96-well plate and left to adhere for 4-6 hours before the addition of Zta-specific T cells. The IncuCyte S3 Live-Cell analysis system was used to analyze the interactions between the T cells and the HDACi-treated gastric cells over a period of 24 hours. The cells were cultured at an E:T ratio of 10:1. The IncuCyte® Cytotox Red Reagent for counting dead cells was added to each well at the start of incubation. The treated AGS-EBV cells were co-cultured with the HLA-A*02:01-restricted TCR4 primary T cell clones and the HLA-mismatched control clone, TCR9. After 24 hours of incubation, the supernatant was removed from the wells for quantification of cytokines. The ability of the clones to kill the target cells was assessed using the automated IncuCyte analysis software. The Cytotox Red apoptosis marker that was added to the wells at the start of co-culture was taken up by dead cells, and the overlap area of this red fluorescence with larger, very bright GFP cells was measured in μm² per replicate well at each set time point.

### Cell Hashing and Single-Cell RNA Sequencing

YCCEL1 single-cell suspensions were collected by trypsinization and washed, and then 1 million cells per treatment condition were resuspended in 100 μL of Cell Staining Buffer (Biolegend). Cell hashing was then performed using TotalSeq-A Hashtag antibodies (Biolegend), following the manufacturer's recommendations for 10× Genomics platforms. Briefly, cell suspensions were incubated with 5 μL of Human TruStain FcX™ Fc Blocking reagent (Biolegend) for 10 minutes at 4 C and then added 1 μg of a specific TotalSeq-A Hashtag antibody to each treatment condition and incubated for 30 minutes at 4 C (see Table 1 for antibodies used). Following three rounds of washing in Cell Staining Buffer, cells were counted and resuspended in PBS with 0.04% BSA. Suspensions were then combined for loading, with one input suspension for 24-hr treatments and one for 48-hr treatments. These suspensions were then loaded onto Chromium Chip B for Chromium 3′ Single Cell Gene Expression profiling (v3, 10× Genomics). scRNA-seq gene expression and antibody hashtag oligo (HTO) libraries were processed as per manufacturers' instructions. Gene expression libraries were sequenced on an Illumina NovaSeq (28 bp Read 1, 8 bp i7 Index, 98 bp Read 2), while the HTO libraries were sequenced on an Illumina NextSeq using the 75-cycle kit (50 bp Read 1, 8 bp i7 Index, 33 bp Read 2).

### Bioinformatic Analyses

A custom reference set for the Cell Ranger scRNA-seq pipeline was first created to allow for the simultaneous analysis of human and EBV genes in scRNA-seq gene expression data. EBV (inverted Akata strain) sequences and gene annotation files extracted from previously published sources (Lin, Z., et al. (2013). Journal of Virology 87, 1172-1182; O'Grady, T., et al. (2016). Nucleic Acids Research 44, e145-e145.) were collected from the public repository (https://github.com/flemingtonlab/public). As Cell Ranger disregards reads marked by the internal aligner as mapping to more than one coding feature, the gene annotation file was modified for regions of the EBV genome where multiple highly overlapping coding regions are present. Specifically, for each of these EBV loci, the overlapping genes were collapsed down into a single “metagene,” representing the union of the overlapping genes as a single feature. These modified EBV reference files were then combined with the GRCh38 (Ensembl 93) pre-built Cell Ranger reference sequence and gene annotation files to create the custom reference set for this study. A UMI count matrix was then generated from the scRNA-seq gene expression data using Cell Ranger v3.1.0 with default arguments. CITE-seq-Count v1.4.3 was used to generate a UMI count matrix from the HTO libraries.

Downstream data analysis was conducted using Seurat v3.1.4 (Stuart, T., et al. (2019). Cell 177, 1888-1902.e1821.) and Monocle 3 v0.2.1 (Qiu, X., et al. (2017). Nature Methods 14, 979-982; Trapnell, C., et al. (2014). Nature Biotechnology 32, 381-386.). Specifically, the Seurat implementation of the MULTI-seq demultiplexing algorithm (McGinnis, C. S., et al. (2019). Nature Methods 16, 619-626.) was used to identify the treatment condition of each single cell from the HTO cell hashing libraries, as well as to remove cross-sample doublets and cells without confident HTO detection. Seurat was then used to do a first-pass clustering and QC, removing low-quality cells (having >=25% mitochondrial UMIs or <=5000 UMI counts) in latent clusters. Seurat was also used to estimate the cell cycle phase of each cell. After this pre-processing, Monocle 3 was then applied for final UMAP dimensionality reduction (McInnes, L., et al. (2018). pp. arXiv: 1802.03426.) clustering, and cell trajectory/pseudotime analysis, after regressing out 25 effects from cell cycle phase and the number of features detected per cell. RNA splicing analysis was conducted using velocyto v0.17.17 (La Manno, et al. (2018). Nature 560, 494-498.). Plots for publication were generated using a combination of Monocle 3, Seurat, and ggplot2 (Wickham, H. (2016). ggplot2—Elegant Graphics for Data Analysis (Springer, Cham)).

### Quantification and Statistical Analysis

All statistical analyses were performed in GraphPad Prism 8 (GraphPad). One-way or two-way ANOVA tests were used to determine the significance of differences between the two groups. P<0.05 were considered significant.

TABLE 1
Key sources

| | | |
|---|---|---|
| REAGENT or | | |
| RESOURCE | SOURCE | IDENTIFIER |

Antibodies

| | | |
|---|---|---|
| Alexa Fluor 488 | Invitrogen | A-11008 |
| anti-rabbit | | |
| Immunoglobulin G | | |
| Alexa Fluor 546 | Invitrogen | A-11030 |
| anti-mouse | | |
| Immunoglobulin G | | |
| Alexa Fluor 647 | Invitrogen | A-21235 |
| anti-mouse | | |
| Immunoglobulin G | | |
| Anti-mouse | Dako | PO161 |
| immunoglobulins/ | | |
| HRP | | |
| Anti-rabbit | Dako | PO217 |
| immunoglobulins/ | | |
| HRP | | |
| CD69 | BioLegend | 302205 |
| HA | Santa Cruz Biotechnology | 912426 |
| Histone H3ac | Active Motif | 39139 |
| (panH3ac) | | |
| P53 | Cell Signaling | 9282 |
| P53 DO1 | Hybridoma | N/A |
| VCA | Abcam | ab48414 |
| Zta | Santa Cruz Biotechnology | sc-53904 |
| β-actin (HRP) | Santa Cruz Biotechnology | sc-47778 |
| TotalSeq-A0251 | BioLegend | 394601 |
| anti-human Hashtag | | |
| 1 Antibody | | |
| TotalSeq-A0252 | BioLegend | 394603 |
| anti-human Hashtag | | |
| 2 Antibody | | |
| TotalSeq-A0254 | BioLegend | 394607 |
| anti-human Hashtag | | |
| 4 Antibody | | |
| TotalSeq-A0255 | BioLegend | 394609 |
| anti-human Hashtag | | |
| 5 Antibody | | |
| TotalSeq-A0257 | BioLegend | 394613 |
| anti-human Hashtag | | |
| 7 Antibody | | |
| TotalSeq-A0258 | BioLegend | 394615 |
| anti-human Hashtag | | |
| 8 Antibody | | |

Chemicals, Peptides, and Recombinant Proteins

| | | |
|---|---|---|
| 12-O- | New England Biolabs | 4174S |
| Tetradecanoylphorbo | | |
| 1-13-Acetate | | |
| 4′,6-Diamidino-2- | Sigma-Aldrich | D8417-5MG |
| phenylindole | | |
| dihydrochloride | | |
| 5-fluorouracil | Sigma-Aldrich | F6627-1G |
| 5′-azacytidine | Sigma-Aldrich | A2385-100MG |
| A366 | SGC, Oxford | N/A |
| Amonafide | Stratech Scientific Ltd | S1367-SEL-10MG |
| BAZ2-ICR | SGC, Oxford | N/A |
| Bromosporine | SGC, Oxford | N/A |
| C646 | SGC, Oxford | N/A |
| Camptothecin | Sigma-Aldrich | C9911-100MG |
| Chidamide | Insight Biotechnology Ltd | Sc-364462 |
| CI-994 | SGC, Oxford | N/A |
| Cisplatin | Sigma-Aldrich | 479306-1G |
| CXD101 | Prof. Nick La Thangue | N/A |
| Daunorubicin | Sigma-Aldrich | D8809-1MG |
| hydrochloride | | |
| Dimethyl sulfoxide | Sigma-Aldrich | D2650-100ML |
| Doxorubicin | Sigma-Aldrich | D1515-10MG |
| hydrochloride | | |
| Entinostat (MS-275) | Stratech Scientific Ltd | S1053-SEL-10MG |
| Epirubicin | Sigma-Aldrich | E9406-10MG |
| hydrochloride | | |
| GSK2801 | SGC, Oxford | N/A |
| GSK484 | SGC, Oxford | N/A |
| GSKJ4 | SGC, Oxford | N/A |
| GSKLSD1 | SGC, Oxford | N/A |
| HDM201 | Selleckchem | S8606 |
| Human TruStain | Biolegend | 442301 |
| FcX | | |
| ICBP112 | SGC, Oxford | N/A |
| Idarubicin | Stratech Scientific Ltd | S1228-SEL-10MG |
| hydrochloride | | |
| Idasanutlin | Cayman | S7205 |
| IOX1 | SGC, Oxford | N/A |
| IOX2 | SGC, Oxford | N/A |
| JQ1 | SGC, Oxford | N/A |
| LAQ824 | SGC, Oxford | N/A |
| LLY-507 | SGC, Oxford | N/A |
| LP99 | SGC, Oxford | N/A |
| Mitoxantrone | Sigma-Aldrich | M6545-10MG |
| Mocetinostat | Stratech Scientific Ltd | S1122-SEL-5MG |
| (MGCD0103) | | |
| NI-57 | SGC, Oxford | N/A |
| Nutlin-3a | Sigma-Aldrich | SML0580-5MG |
| OF-1 | SGC, Oxford | N/A |
| OICR-9429 | SGC, Oxford | N/A |
| Olaparib | SGC, Oxford | N/A |
| Panobinostat | Cayman Chemicals | 13280-5MG |
| PFI-1 | SGC, Oxford | N/A |
| PFI-2 | SGC, Oxford | N/A |
| PFI-3 | SGC, Oxford | N/A |
| PFI-4 | SGC, Oxford | N/A |
| PXD101 | Cambridge Bioscience | 2480-5 |
| Romidepsin | Cambridge Bioscience | 17130-500 μg-CAY |
| SAHA | Sigma-Aldrich | SML0061-5MG |
| SGC-CBP30 | SGC, Oxford | N/A |
| SGC0946 | SGC, Oxford | N/A |
| SGC707 | SGC, Oxford | N/A |
| Teniposide | Sigma-Aldrich | SML0609-1MG |
| Topotecan | Cambridge Bioscience | 14129-25MG-CAY |
| Trichostatin A | Sigma-Aldrich | T8552-1MG |
| UNC0638 | SGC, Oxford | N/A |
| UNC0642 | SGC, Oxford | N/A |
| UNC1215 | SGC, Oxford | N/A |
| UNC1999 | SGC, Oxford | N/A |

Critical Commercial Assays

| | | |
|---|---|---|
| Chromium Single | 10x Genomics | 1000075; 1000078 |
| Cell Gene | | |
| Expression, 3′, v3 | | |
| chemistry | | |

Deposited Data

| | | |
|---|---|---|
| YCCEL1 raw single- | This study | GEO: [accession] |
| cell gene expression | | |
| and hashtag oligo | | |
| sequencing data, and | | |
| R object containing | | |
| processed data | | |
| Human (hg38) + | This study | GEO: [accession] |
| EBV reference set | | |
| EBV sequence and | Lin, Z., et al. (2013). Journal | https://github.com/flemingtonlab/ |
| gene annotation files | of Virology 87, 1172-1182; | public |
| (Akata strain, | O'Grady, T., et al. (2016). | |
| inverted) | Nucleic Acids Research 44, | |
| | e145-e145. | |

Experimental Models: Cell Lines

| | | |
|---|---|---|
| AGS rEBV-GFP | Stewart, S., et al. (2004). Proc | N/A |
| | Natl Acad Sci U S A 101, | |
| | 15730-15735. | |
| Akata | Takada, K., et al. (1991). | N/A |
| | Virus Genes 5, 147-156. | |
| C666.1 | Cheung, S. T., et al. (1999). | N/A |
| | International Journal of | |
| | Cancer 83, 121-126. | |
| Namalwa | Klein, G., et al. (1972). | N/A |
| | International Journal of | |
| | Cancer 10, 44-57. | |
| OE19 rEBV-GFP | Stewart, S., et al. (2004). Proc | N/A |
| | Natl Acad Sci U S A 101, | |
| | 15730-15735. | |
| PD LCL | Takada, K., et al. (1991). | N/A |
| | Virus Genes 5, 147-156. | |
| Raji | Pulvertaft, R. J. V. (1964). The | N/A |
| | Lancet 283, 238-240. | |
| SNU-NCC-24 | Korean Cell Line Bank | 50024 |
| SNU719 | Park, J.-G., et al. (1997). | N/A |
| | International Journal of | |
| | Cancer 70, 443-449. | |
| YCCEL1 | Kim, D. N., et al. (2013). | N/A |
| | Journal of General Virology | |
| | 94, 497-506. | |

Oligonucleotides

| | | |
|---|---|---|
| qPCR primers - see | This study | N/A |
| Table S1 | | |
| siRNAs - see Table | This study | N/A |
| S1 | | |

Recombinant DNA

| | | |
|---|---|---|
| pcDNA3-FlagHA- | Bailey, D., and O'Hare, P. | N/A |
| SENP1-C603S | (2004). Journal of Biological | |
| | Chemistry 279, 692-703. | |
| pcDNA3-FlagHA- | Bailey, D., and O'Hare, P. | N/A |
| SENP1-WT | (2004). Journal of Biological | |
| | Chemistry 279, 692-703. | |
| pcDNA3-HA-p53- | Smirnov A, et al. Aging | N/A |
| WT | (Albany NY). 2018; 10: 3308- | |
| | 3326. | |

Software and Algorithms

| | | |
|---|---|---|
| Flowjo X | Flowjo LLC. | N/A |
| ggplot2 | Wickham, H. (2016). ggplot2 - | https://ggplot2.tidyverse.org/ |
| | Elegant Graphics for Data | |
| | Analysis (Springer, Cham). | |
| GraphPad Prism 8 | GraphPad Software | N/A |
| Harmony | PerkinElmer | N/A |
| Cell Ranger v3.1.0 | 10x Genomics | https://support.10xgenomics.com/ |
| | | single-cell-gene-expression/ |
| | | software/downloads/latest |
| CITE-seq-Count | https://github.com/Hoohm/CITE- | https://github.com/Hoohm/CITE- |
| v1.4.3 | seq-Count | seq-Count |
| Seurat v3.1.4 | Stuart, T., et al. (2019). Cell | https://satijalab.org/seurat/ |
| | 177, 1888-1902.e1821. | |
| Monocle 3 v0.2.1 | Qiu, X., et al. (2017). Nature | https://cole-trapnell- |
| | Methods 14, 979-982. | lab.github.io/monocle3/ |
| velocyto v0.17.17 | La Manno, G., et al. (2018). | http://velocyto.org/ |
| | Nature 560, 494-498. | |
| R v3.6.1 | The R Foundation for | https://www.r-project.org/ |
| | Statistical Computing | |
| Python v3.8.2 | Python Software Foundation | https://www.python.org/ |

TABLE 2
Primers

| | | | SEQ ID NO |
|---|---|---|---|
| RT-qPCR | | | |
| GAPDH | For | 5′- GCCTCCTGCACCACCAACTG -3′ | 1 |
| | Rev | 5′- CGACGCCTGCTTCACCACCTTCT -3′ | 2 |
| BZLF1 | For | 5′- GCACATCTGCTTCAACAGGA -3′ | 3 |
| | Rev | 5′- CCAAACATAAATGCCCCATC -3′ | 4 |
| BRLF1 | For | 5′- CCTGTCTTGGACGAGACCAT -3′ | 5 |
| | Rev | 5′- AAGGCCTCCTAAGCTCCAAG -3′ | 6 |
| BBLF4 | For | 5′- AAGCCTGCCTCATCCTTGACC -3′ | 7 |
| | Rev | 5′- GACGAGCCTCTCCTTCACGG -3′ | 8 |
| BGLF5 | For | 5′- TTCGGCCGCTATTAGCTTAG -3′ | 9 |
| | Rev | 5′- GACGGGGGAATAATCAACCT -3′ | 10 |
| BMRF1 | For | 5′- CGTGCCAATCTTGAGGTTTT -3′ | 11 |
| | Rev | 5′- CGGAGGCGTGGTTAAATAAA -3′ | 12 |
| BcLF1 | For | 5′- CCTCCCTGACCGTTCCCAG -3′ | 13 |
| | Rev | 5′- GCAGTTTGAGACCGCCACATC -3′ | 14 |
| BLLF 1 | For | 5′- TGGCGAGTTTGCGTCCTCAG -3′ | 15 |
| | Rev | 5′- CGTCCAGTGTCACGATTTCTTGG -3′ | 16 |
| BGLF4 | For | 5′- TCGCGTTTTCGAAAGAAGGC -3′ | 17 |
| | Rev | 5′- TCTACGTAATGACGGACCCA -3′ | 18 |
| qPCR for EBV genome load | | | |
| BALF5 | For | 5′- CTTTGGCGCGGATCCTC -3′ | 19 |
| | Rev | 5′- AGTCCTTCTTGGCTAGTCTGTTGAC -3′ | 20 |
| | probe | 5′- CATCAAGAAGCTGCTGGCGGCC -3′ | 21 |
| B2M | For | 5′- GGAATTGATTTGGGAGAGCATC -3′ | 22 |
| | Rev | 5′- CAGGTCCTGGCTCTACAATTTACTAA -3′ | 23 |
| | probe | 5′- AGTGTGACTGGGCAGATCATCCACCTTC -3′ | 24 |
| ChIP-qPCR primers | | | |
| CDKN1_ | For | 5′- GTTGGGACATGTTCCTGACGG -3′ | 25 |
| promoter | Rev | 5′- CTCCCTCCATCCCTATGCTGC -3′ | 26 |
| BZLF1_ | For | 5′- GCGAGAGGTGTGTCAGCCAA -3′ | 27 |
| promoter | Rev | 5′- TGGGAGCCAAAGAGGCAGG -3′ | 28 |
| BZLF1_ | For | 5′- TCCAGTGGGGTAAATGCACCT -3′ | 29 |
| enhancerRev | | 5′- AAGATAGCATGGCCGTGGGG -3′ | 30 |
| KRT14_ | For | 5′- AGGAAGTTGAGGGCGTTCTG -3′ | 31 |
| promoter | Rev | 5′- GGCCCACATTTGAGAGGTCA -3′ | 32 |
| HTO primers (scRNA seq) | | | |
| D701_s primer, | | CAAGCAGAAGACGGCATACGAGATCGAGTAA | 33 |
| i7 index | | TGTGACTGGAGTTCAGACGTGTGC | |
| D702_s primer, | | CAAGCAGAAGACGGCATACGAGATTCTCCGG | 34 |
| i7 index | | AGTGACTGGAGTTCAGACGTGTGC | |
| HTO additive | | GTGACTGGAGTTCAGACGTGTGCTC | 35 |
| primer | | | |
| SI-PCR primer | | AATGATACGGCGACCACCGAGATCTACACTCT | 36 |
| | | TTCCCTACACGACGCTC | |

### Example 2

### Benzamide-Based HDACi Preferentially Reactivates EBV in Gastric Epithelial But Not B Cells

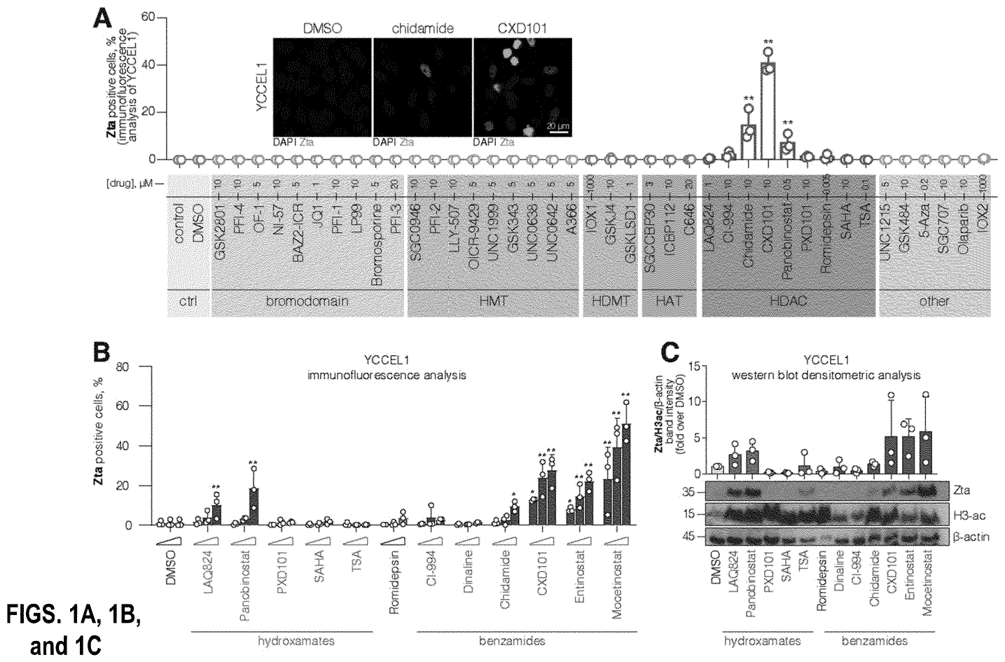

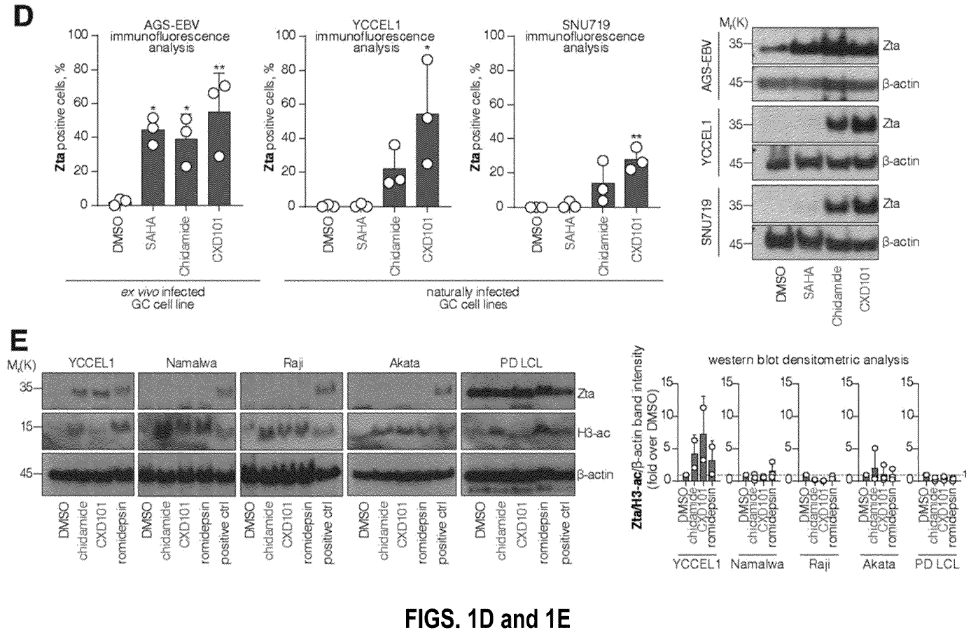

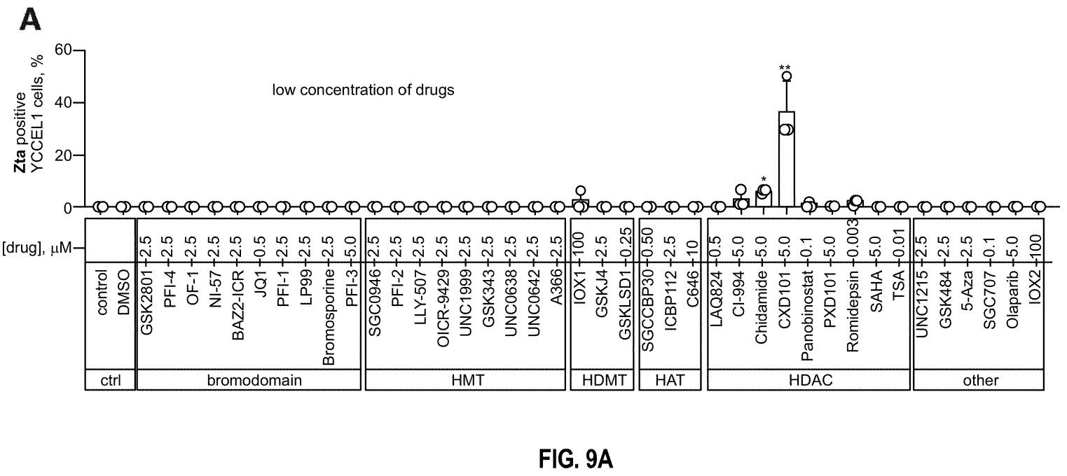

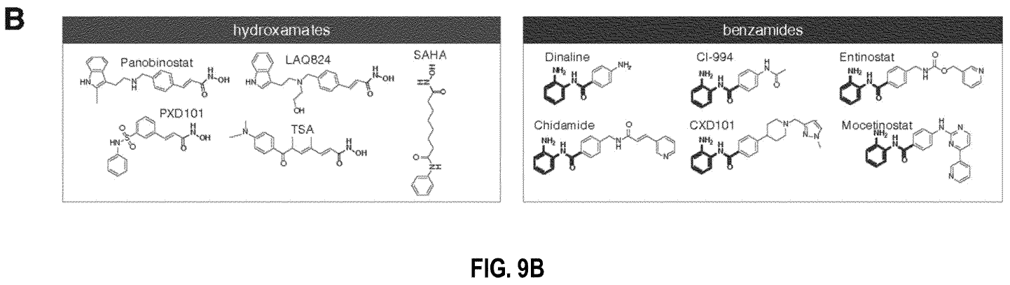

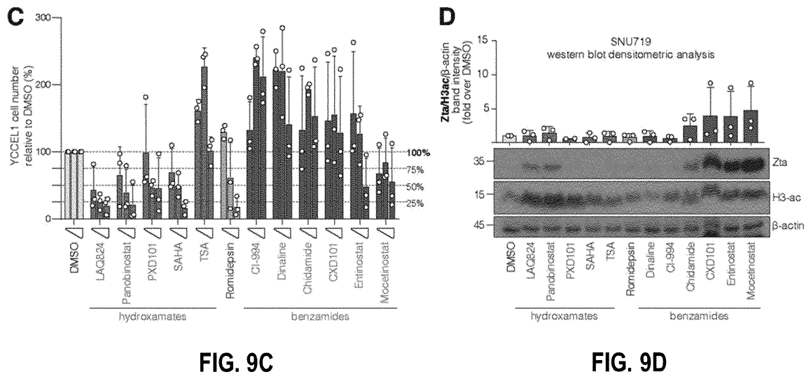

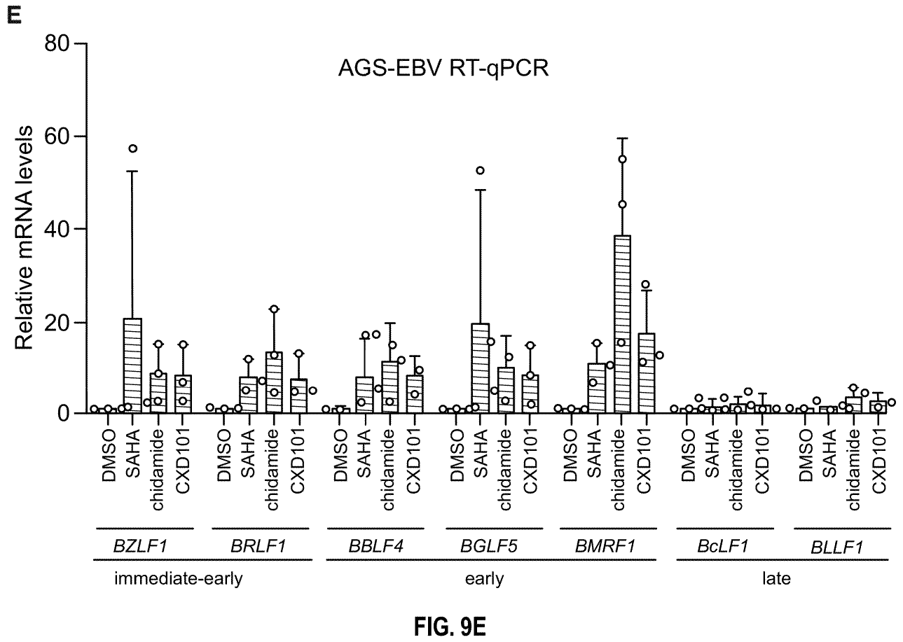

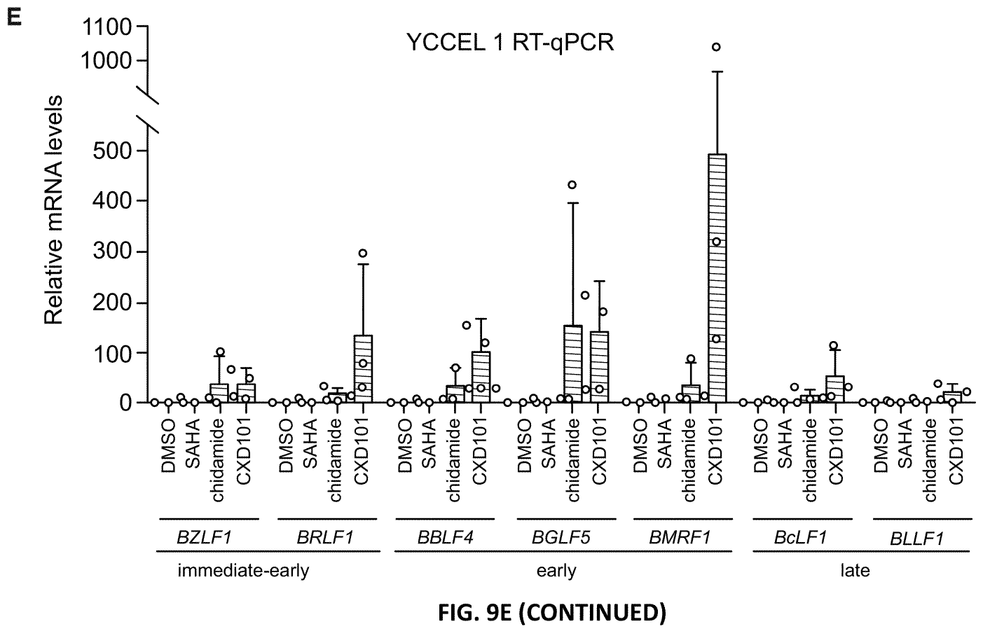

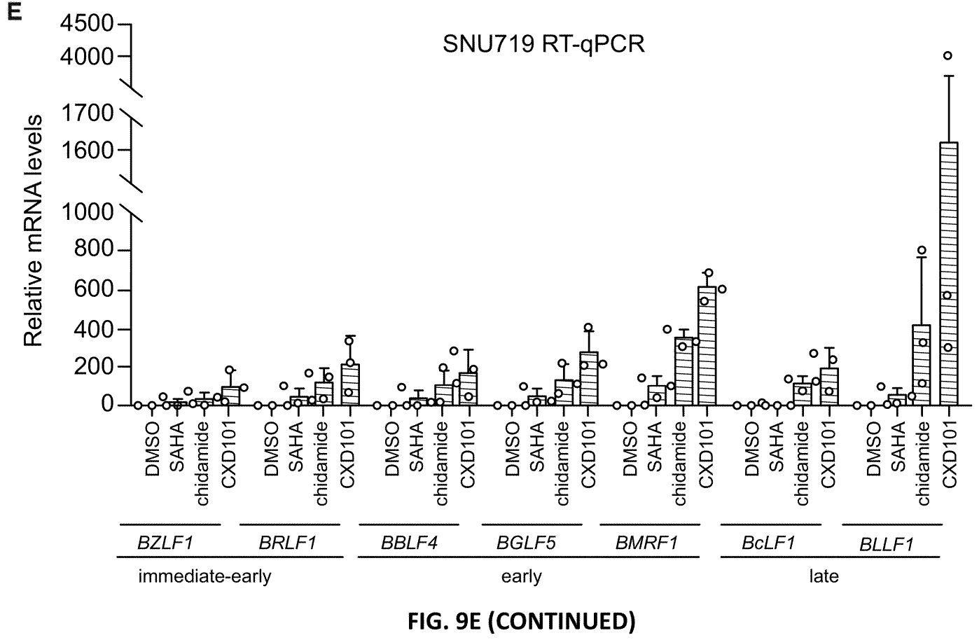

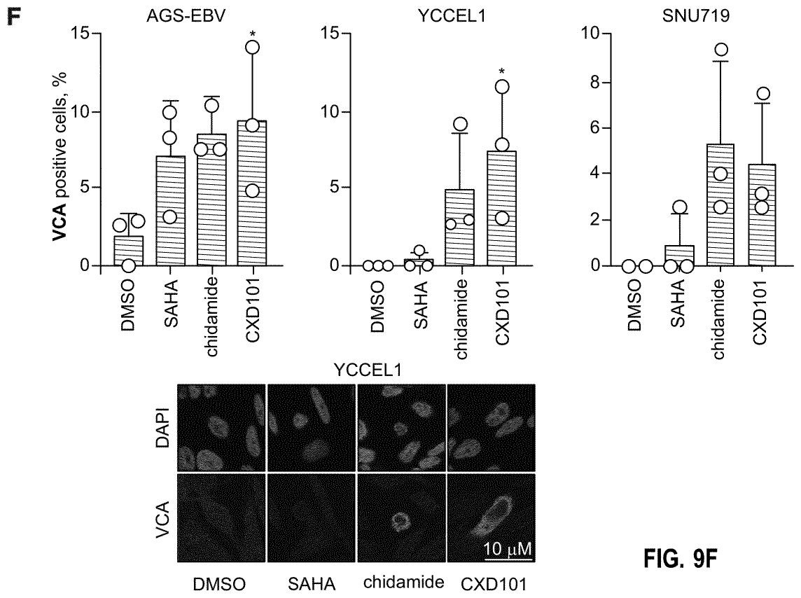

To identify compounds able to reverse latency in the naturally EBV-infected gastric cancer cell line YCCEL1, high content cell imaging assay was used to screen a library of 40 epigenetic drugs (FIG. 1A), including inhibitors of bromodomains, histone methyltransferases (HMT), histone demethylases (HDMT), histone acetyltransferases (HAT) and HDACs. The readout was percentage of cells positive for the immediate-early viral transcription factor Zta, as assessed by immunofluorescence staining and confocal microscopy. The only group that increased the number of nuclear Zta-positive cells (>5% positive cells), both at low (FIG. 9A) and high (FIG. 1A) drug concentrations, were HDACi. Two hits from the HDACi group, chidamide (Zta+ cells: 5.8% at 5 μM and 14.5% at 10 μM) and CXD101 (Zta+ cells: 36.5% at 5 μM and 40.7% at 10 μM) are benzamide-based HDACi, which are structurally different from the commonly used hydroxamate-based HDACi, such as SAHA (FIG. 9B). YCCEL1 cells were subsequently exposed to increasing concentrations of indicated five hydroxamate-based and six benzamide-based HDACi, as well as romidepsin, whose structure is different from both groups. Among the hydroxamates, LAQ824 and panobinostat were able to reactivate EBV, but showed high toxicity (FIGS. 1B and 9C). The benzamide-based HDACi chidamide, CXD101, entinostat, and mocetinostat showed a strong dose-dependent effect on Zta expression, and chidamide and CXD101 did so with low toxicity (FIGS. 1B and 9C). Western blot analysis of Zta and total histone 3 acetylation (H3-ac) expression showed that Zta levels normalized over H3-ac were higher in benzamide HDACi-treated cells compared with hydroxamate HDACi treatment (FIG. 1C). Benzamide HDACi-induced Zta expression also occurred in another widely used naturally-infected EBV+GC cell line, SNU719 (FIG. 9D). The failure of hydroxamate-based HDACi, such as PXD101 and SAHA, or romidepsin, to induce Zta expression is not due to a lack of HDACi activity as all three induced detectable H3-ac (FIGS. 1C and 9D). In contrast to the naturally infected cells YCCEL1 and SNU719, treatment of the ex-vivo infected GC cell line AGS-EBV (Stewart, S., et al. (2004). Proc Natl Acad Sci USA 101, 15730-15735) with any of SAHA, chidamide or CXD101 led to strong activation of Zta expression (FIG. 1D).

Although entinostat and mocetinostat induced Zta expression at higher levels than other benzamides, they resulted in high toxicity at high concentrations (FIGS. 1B and 9C). CXD101 is currently undergoing clinical trials (Eyre, T. A., et al. (2019). Cancer 125, 99-108.), and chidamide is approved in China by the National Medical Products Administration (NMPA) for treatment of peripheral T-cell lymphoma (Xu, Y., et al. (2017). Drugs of Today; 53.) and breast cancer (Jiang, Z., et al. (2018). Annals of Oncology; 29.). Therefore, chidamide and CXD101 were used to treat naturally-infected Burkitt lymphoma cell lines Namalwa, Raji, and Akata, or the ex vivo infected lymphoblastoid cell line PD-LCL in parallel with YCCEL1 cells. Romidepsin was used to treat the same panel of cells. Remarkably, none of these HDACi induced Zta expression in naturally infected Burkitt lymphoma cell lines. Zta expression level in ex vivo infected B cell line PD-LCL is detectable in DMSO treated cells and it is not affected after HDACi treatment. All HDACis used induced detectable amounts of H3-ac, with cell line-dependent variation (FIG. 1E). To assess the extent of EBV reactivation, mRNA expression of several immediate-early, early, and late lytic genes was analysed (FIG. 9E). In YCCEL1 cells, lytic gene expression was 2 to 50 times higher in chidamide- or CXD101-treated cells compared with SAHA treatment, and a similar trend was observed in SNU719 cells. In ex vivo-infected AGS-EBV cells, all three drugs led to a comparable activation of lytic genes (FIG. 9E). Up to 5-10% of YCCEL1 and SNU719 cells were positive for late Viral Capsid Antigen (VCA) after chidamide or CXD101 treatment, but not SAHA-treatment. In AGS-EBV cells, all three treatments led to similar VCA expression levels (FIG. 9F). Both benzamide- and hydroxymate-based HDACi can induce EBV reactivation in gastric epithelial cancer cells, but benzamide-based HDACis are generally less toxic than hydroxymate-based HDACi. Under the same conditions, none of the tested HDACi were able to reactivate EBV in naturally- or ex vivo-infected B cell lines. Together, these results indicate that HDACi, such as benzamide-based HDACi, predominantly induce EBV reactivation in gastric epithelial cancer cells but not in B cells.

### Example 3

Topoisomerase Inhibitors Synergize with HDACi to Reactivate EBV With High Efficiency

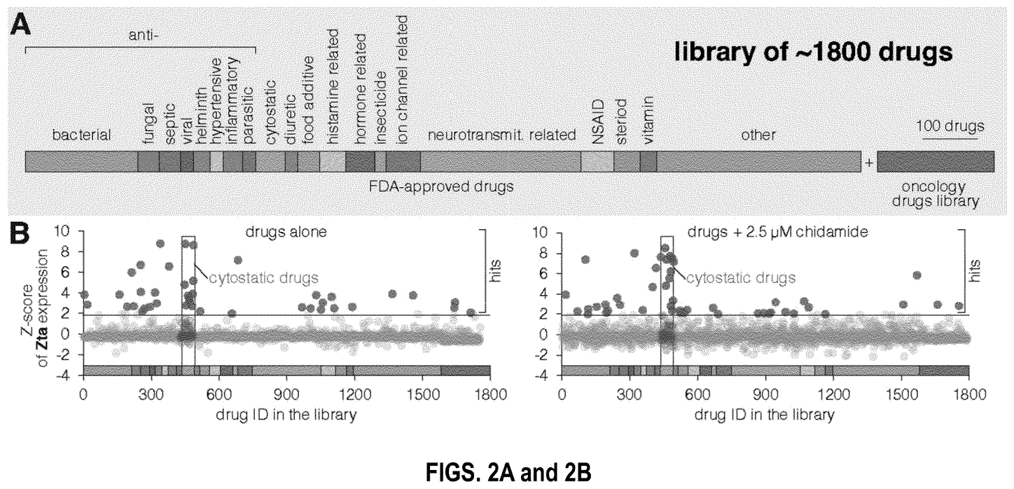

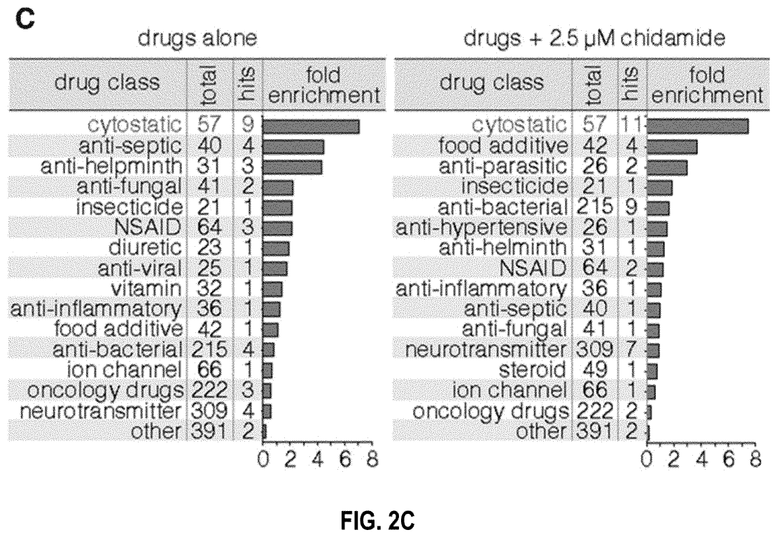

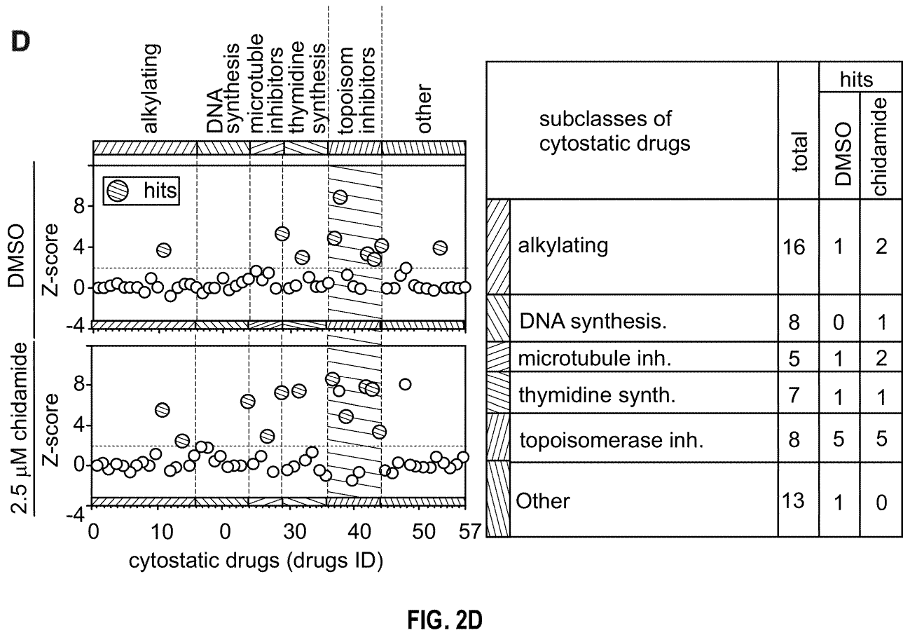

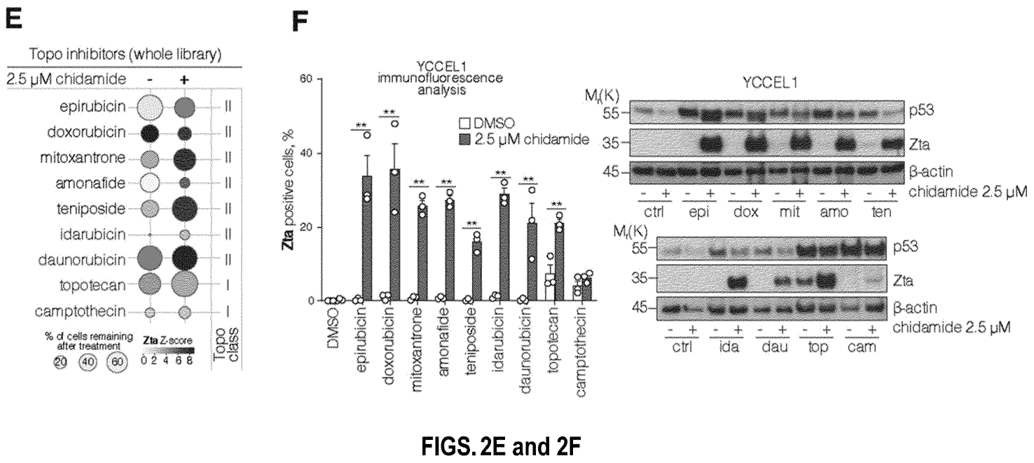

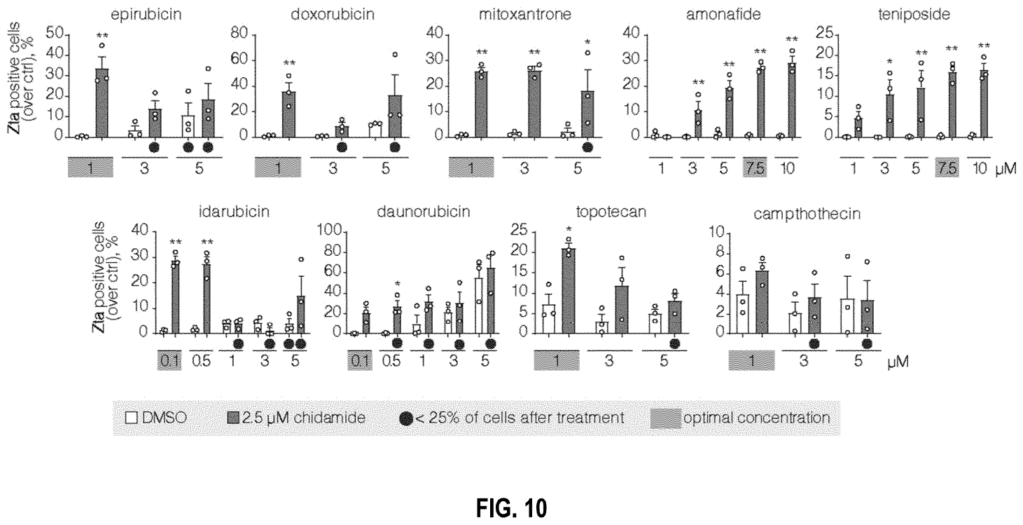

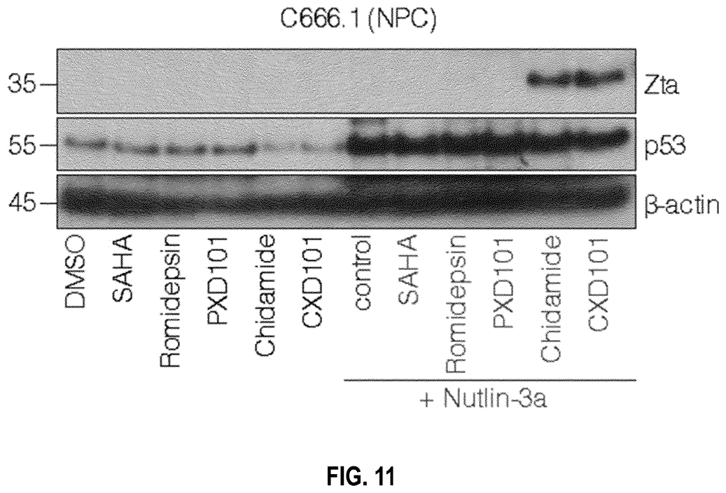

To enhance the efficacy of EBV reactivation, a library of 1600 FDA-approved drugs and 222 approved and experimental oncology drugs (FIG. 2A) was screened to identify any that synergize with chidamide, one of the benzamide HDACi with low toxicity, to induce Zta expression in YCCEL1 cells. A low concentration of chidamide (2.5 μM) was used, which alone reactivates EBV in less than 1% of cells, and used the percentage of Zta-positive cells normalized over the average value across all samples (Z-score) as the readout. Using a threshold of Z>1.96 and cell number >1,000 cells, 41 and 47 hits were identified in “drugs alone” and “drugs with chidamide” conditions, respectively (FIG. 2B). In both conditions, hits were enriched for cytostatic drugs (FIGS. 2B-C). Among subclasses of cytostatic drugs, topoisomerase inhibitors were the most enriched subclass in both conditions (⅝ topoisomerase inhibitors were hits) (FIG. 2D). To assess all topoisomerase inhibitors in the library, all screened drugs were filtered according to PubChem classification and 9 (mostly topoisomerase II inhibitors) were identified, most of which showed higher Z-scores with chidamide co-treatment (FIG. 2E). However, some had Z-scores below the threshold or high toxicity (<20% cells compared with control) (FIG. 2E). Topoisomerase inhibitor treatment conditions were optimized to achieve high levels of EBV reactivation with minimal toxicity (FIG. 10A). At optimal doses, 8 out of 9 topoisomerase inhibitors showed high synergy with a low concentration of chidamide, leading to 20-40% of cells expressing high levels of Zta (FIG. 2F). The ability of topoisomerase II inhibitors to synergize with chidamide is of great importance as many topoisomerase inhibitors are first-line chemotherapeutic agents, potent inducers of DNA damage and p53 (FIG. 2F). Notably, epirubicin is used to treat gastric cancer.

### Example 4

### Enhanced p53 Activity Synergizes With HDACi to Induce EBV Latency Reversal

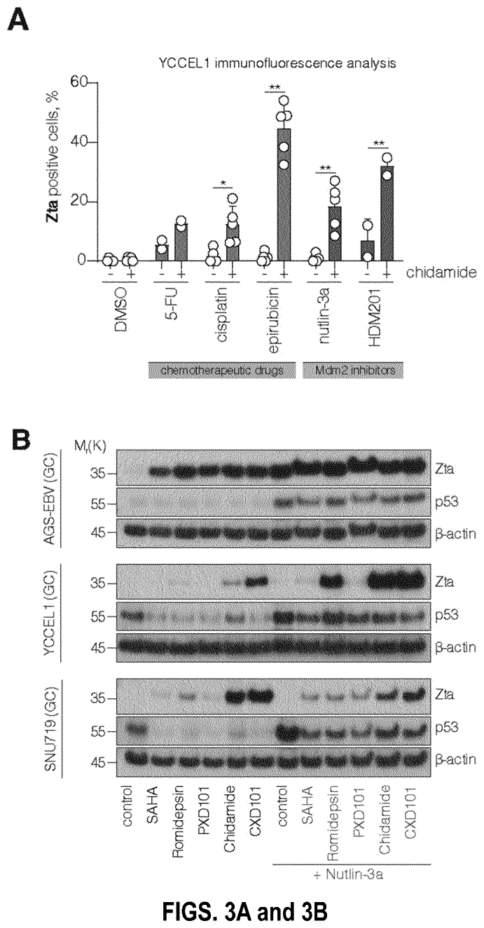

Most EBV+GC cells contain wild type p53, but the role of p53 in the EBV latent-lytic cycle switch is unclear. Since p53 is highly induced by topoisomerase inhibitors, whether enhanced p53 activity is able to synergize with HDACi to induce EBV reactivation was tested. p53 activity was induced either by Mdm2 inhibitors (Mdm2i), such as nutlin-3a and HDM201, that stabilize p53 without DNA damage or by chemotherapeutic drugs (5-fluorouracil (5-FU), cisplatin or epirubicin) that are used to treat gastric cancer in the UK. These chemotherapeutic drugs damage DNA via different mechanisms. Low concentration of chidamide (2.5 μM), Mdm2i, or the chemotherapeutic drugs alone induced Zta expression in less than 5% of cells. In contrast, strong Zta expression was detected in ˜15% of cells treated with chidamide combined with 5-FU or cisplatin, and up to 50% of cells treated with chidamide combined with epirubicin (FIG. 3A). Importantly, the combination of chidamide and Mdm2i also induced Zta expression in up to ˜35% of treated cells. As Mdm2i stabilize p53 without damaging DNA, these results indicate that stabilization of p53 is sufficient to enhance chidamide-induced Zta expression, indicative of EBV latency reversal (FIG. 3A).

The ability of nutlin-3a to synergize with a panel of HDACi to induce Zta expression was tested in a number of EBV+ epithelial cancer cell lines. As shown in FIG. 3B, all tested HDACi or nutlin3a alone were able to induce high levels of Zta expression in AGS-EBV cells and the combination treatment with nutlin-3a only resulted in a modest further increase in Zta expression. In YCCEL1 and C666.1, the EBV naturally infected nasopharyngeal cancer (NPC) cell lines, nutlin-3a enhanced the ability of benzamide-based HDACi chidamide and CXD101 but not hydroxymate-based HDACi SAHA or PXD101 to induce Zta. In SNU719 cells, however, Zta expression is slightly higher in cells treated with SAHA or PXD101 combined with nutlin-3a compared to single-agent treated cells. Importantly, nutlin-3a failed to synergize with chidamide or CXD101 to induce Zta expression. Under the same conditions, romidepsin's ability to induce Zta expression was most clearly enhanced by nutlin-3a in YCCEL1 cells. These results show that nutlin-3a induced p53 expression synergizes better with benzamide-based HDACi than with hydroxymate-based HDACi in inducing Zta expression in EBV naturally infected epithelial cancer cells. However, the extent of such synergy is cell context-dependent.

### Example 5

### Wild Type p53 is Required for EBV Reactivation in EBV+ Epithelial Cancer Cells

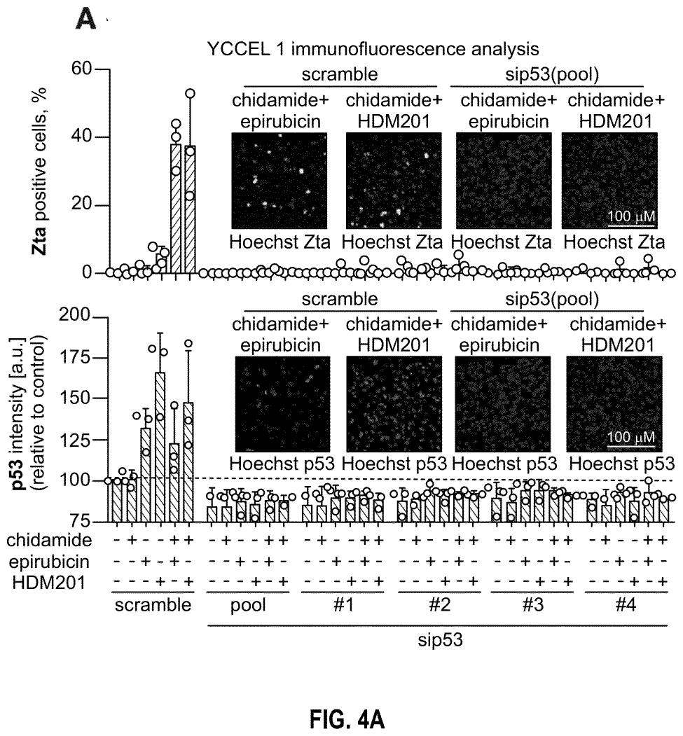

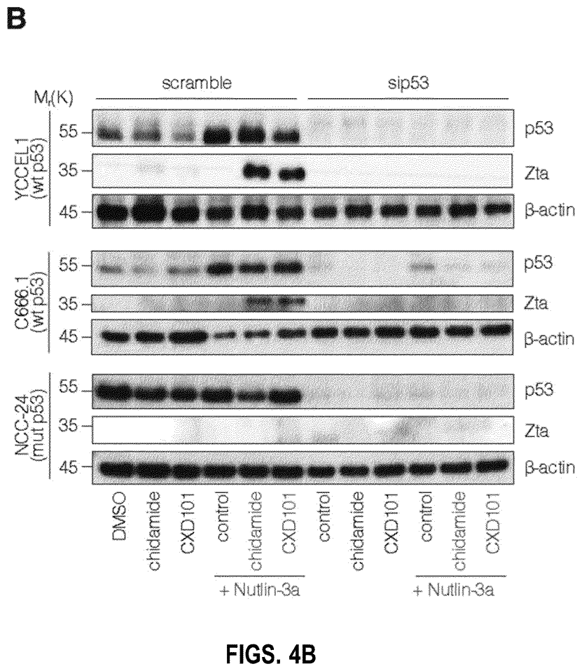

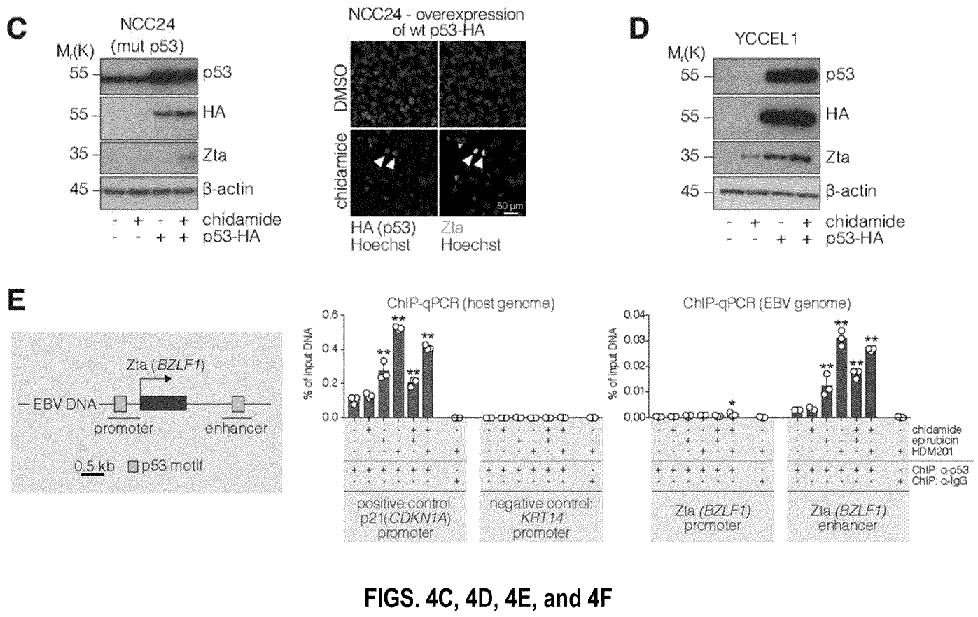

To test the requirement for p53 in EBV reactivation, RNAi-mediated knockdown of p53 was carried out using four different siRNAs alone or in combination (pool). Induction of Zta expression upon treatment with chidamide combined with epirubicin or HDM201 was completely abolished after p53 RNAi (FIG. 4A). These data were confirmed by Western blot in YCCEL1 and C666.1 cells, which express wild type p53, treated with nutlin-3a alone or in combination with chidamide or CXD101 (FIG. 4B). Consistent with this observation, the gastric cancer cell line NCC-24, which harbors mutant TP53 (G266R and R273H), failed to express Zta under the same treatments (FIG. 4B). This defect was restored in NCC-24 cells by introducing HA-tagged wild-type p53 followed by chidamide treatment, which resulted in the induction of Zta expression (FIG. 4C). Overexpression of HA-tagged wild-type p53 in YCCEL1 cells further potentiated chidamide-induced Zta expression (FIG. 4D).

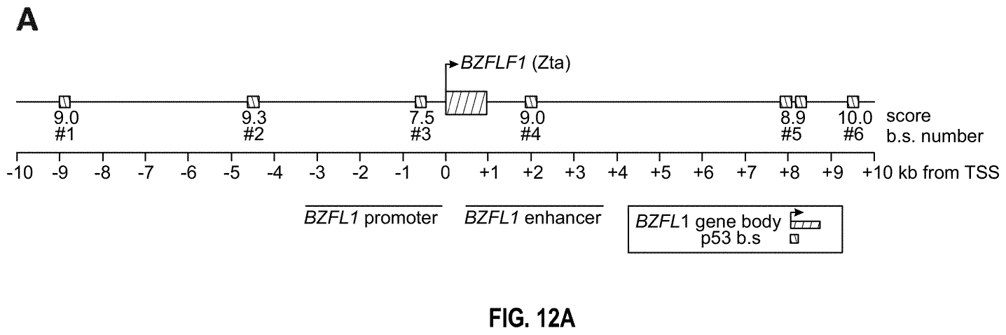

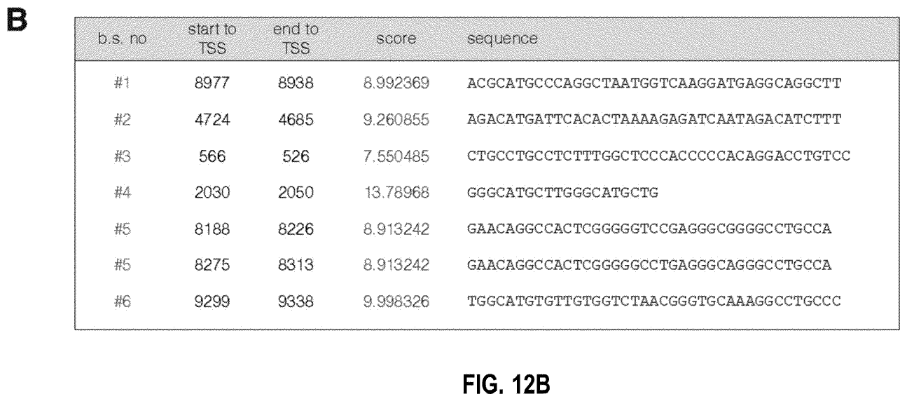

To investigate whether p53 can directly regulate Zta expression, p53 binding motifs were searched in a 20 kb region around the BZLF1 transcription start site (TSS) (FIGS. 12A-B) (Kouwenhoven, E. N., et al. (2010). PLOS Genet 6, e1001065), and two potential p53 binding sites were identified, a canonical and non-canonical p53 binding sites in the potential enhancer and promoter region of BZLF1 approximately 2 kb downstream or 500 bp upstream of BZLF1 TSS (b.s. #4 and b.s. #3, respectively, FIGS. 12A-B). The ability of p53 to bind either of the identified binding sites was tested using chromatin immunoprecipitation (ChIP). The CDKN1A and KRT14 promoters were used as positive and negative controls for p53 binding, respectively. Under the same ChIP conditions, more p53 binding was detected at the enhancer region than at the promoter region of BZLF1 (FIG. 4E). p53 binding at the BZLF1 enhancer was further increased upon p53 stabilization by HDM201 or epirubicin alone or combined with chidamide (FIG. 4E). These findings show that p53 is required for EBV reactivation in the cell lines tested and that p53 may induce Zta expression by binding to the BZLF1 enhancer via a previously unrecognized p53 binding site.

### Example 6

### Single-Cell Sequencing Reveals Treatment-Dependent Distinctive EBV Reactivation States

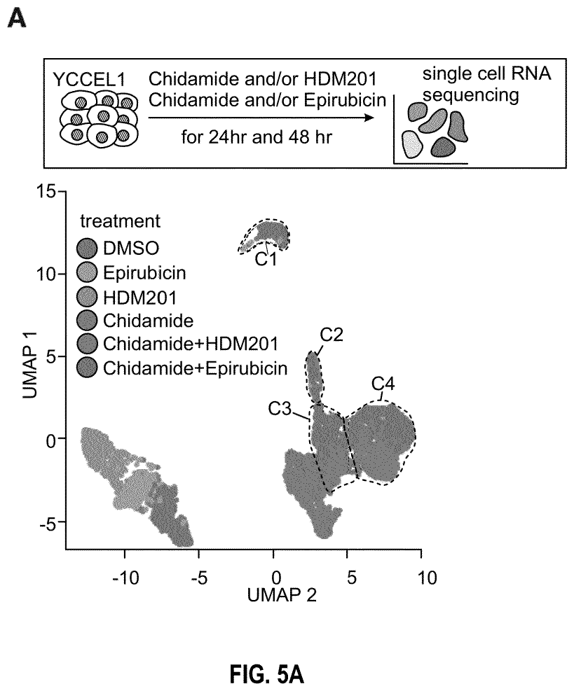

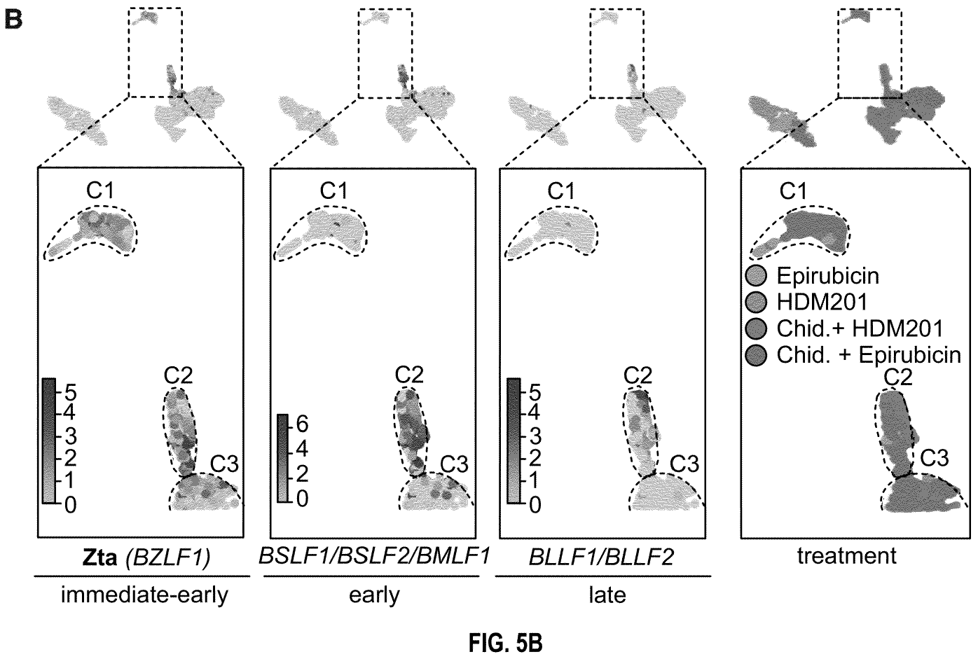

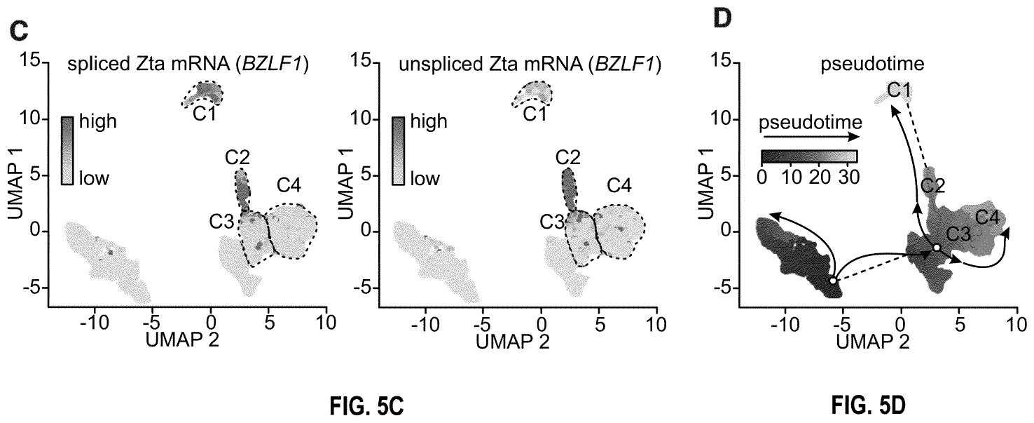

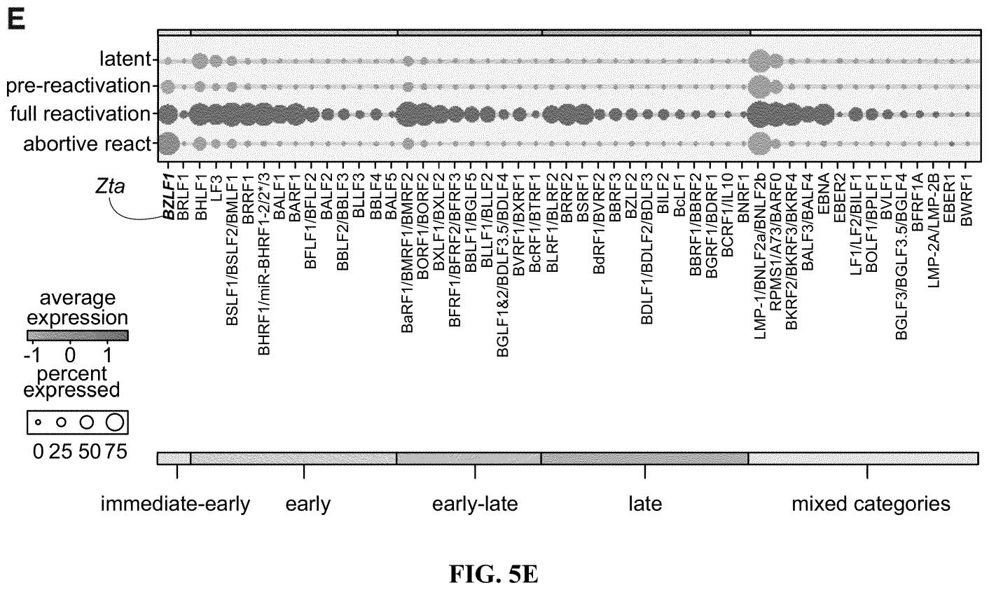

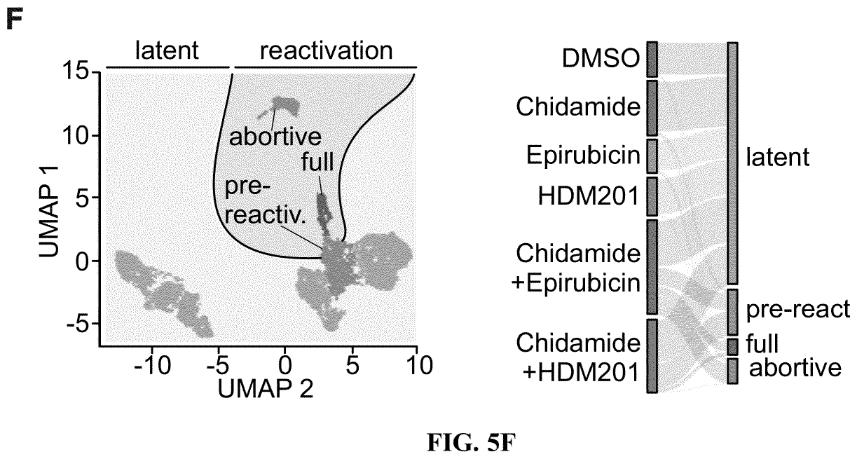

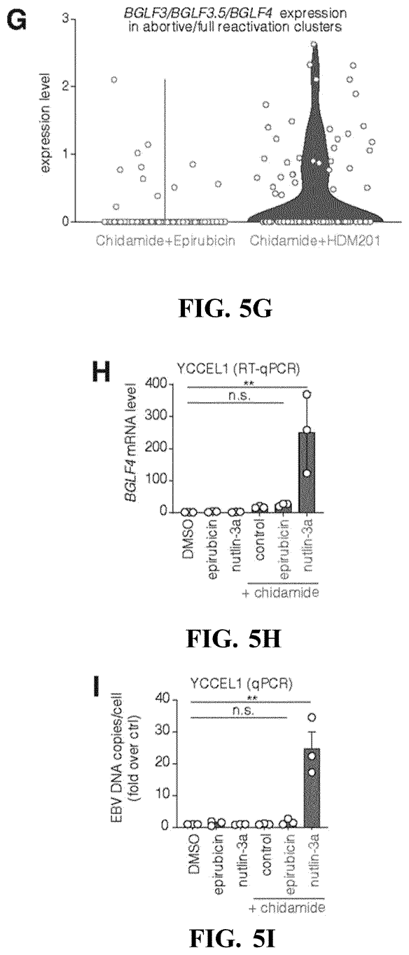

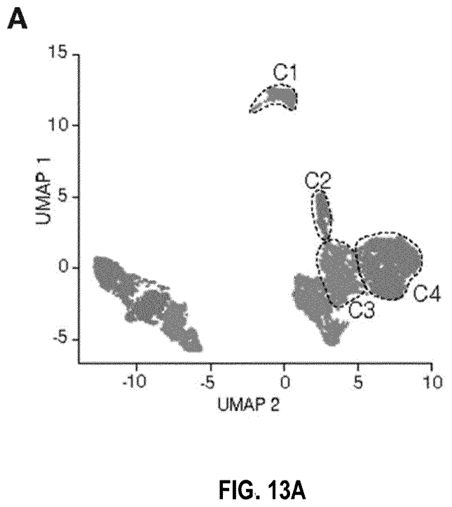

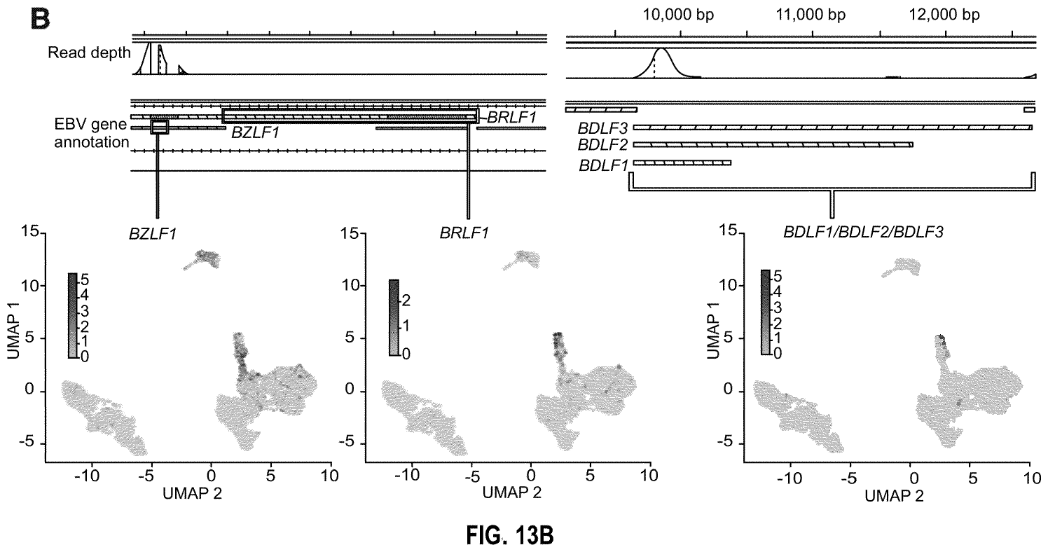

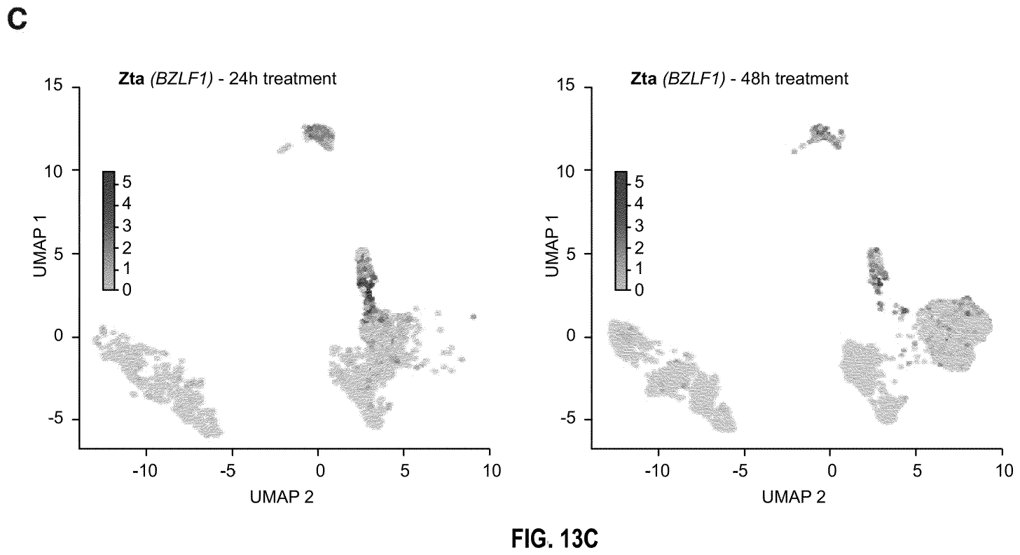

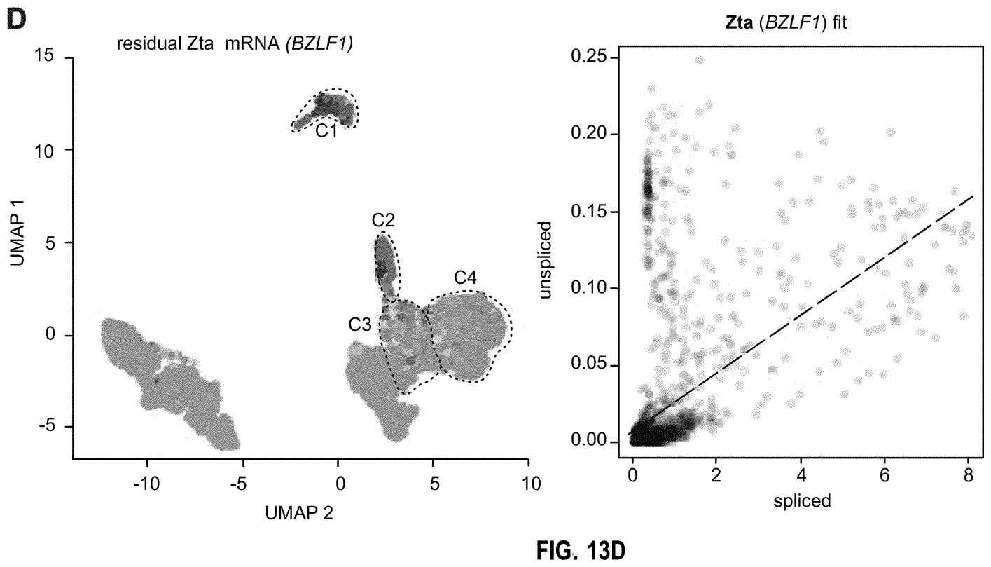

Given the highly variable rates of Zta expression induced by different drug combinations in cell lines such as YCCEL1, single-cell RNA sequencing (scRNA-seq) was conducted to investigate why some cells experience EBV reactivation in response to a defined treatment but not others and to characterize various states of EBV reactivation that may exist in subpopulations of treated cells. scRNA-seq data were generated and analyzed for 7,595 YCCEL1 cells treated with epirubicin, HDM201, or DMSO control, alone or in combination with chidamide, for 24 h and 48 h. Uniform Manifold Approximation and Projection (UMAP) dimensionality reduction and clustering showed that cells treated with single agents (e.g., DMSO, chidamide, epirubicin, or HDM201 alone) largely fell into discrete clusters unique to each treatment, whereas cells treated with combination treatments (chidamide combined with epirubicin or chidamide combined with HDM201) largely fell into one of four shared clusters, labeled as C1-C4, respectively (FIGS. 5A and 13A).

The expression of EBV genes in the scRNA-seq data was next assessed. To optimize quantification for EBV loci where individual gene expression could not be resolved due to the presence of multiple highly overlapping genes, the overlapping genes were collapsed into gene aggregates representing the union of the individual genes. Genes with resolvable overlap were kept distinct; for example, for the BZLF1 gene (encoding Zta), the unique second exon allowed for resolving its expression from that of the overlapping BRLF1 gene (FIG. 13B). Using this approach, expression of 25 individual EBV genes and 26 gene aggregates comprised of two or more overlapping EBV genes was detected.

The expression of the immediate-early gene Zta, a commonly used marker of EBV reactivation, was first examined. High expression of Zta in C1, C2, and a part of C3 clusters that were mainly derived from combination treatments was observed. Additionally, EBV gene aggregates containing the early genes BLF1/BSLF2/BMLF1 or late genes BLLF1/BLLF2 were also highly expressed in C2, but were not highly expressed in C1, demonstrating that distinctive EBV reactivation states are present among the Zta-high populations induced by latency reversal agents (FIGS. 5B and 13C).

The expression of Zta but not early or late genes in C1 initially suggested that this cluster represented an intermediate state of reactivation. However, when quantifying the level of spliced and unspliced Zta RNA, it was found that the relative abundance of spliced compared to unspliced Zta RNA was much higher in the C1 cluster than in C2. This argued against identifying C1 as an intermediate reactivation state, indicating that C1 cells may actually be further away from the point of initial Zta transcription induction than the C2 cells expressing EBV early and late genes (FIGS. 5C and 13D). To further confirm these findings, a cell trajectory and pseudotime analysis was conducted. The reconstructed trajectory indicated that combination-treated cells reach a branch point within C3, after which the trajectory branches progress either towards Zta-high states on the edge of C3 and into C2 and C1, or towards a latent Zta-low phenotype in C4. Cells appeared to reach this branch point in C3 mainly by the 24 hr time point, whereas, by the 48 hr time point, cells had progressed almost entirely towards their Zta-high or Zta-low endpoints. Consistent with the Zta splicing analysis, the pseudotime analysis revealed that C1 was further along the reconstructed cellular trajectory than C2 (FIGS. 5D and 13E-F). These data are consistent with a full reactivation state in C2, and an incomplete or “abortive” EBV reactivation phenotype in C1, where the full suite of EBV lytic cycle genes are not expressed despite high levels of Zta.

Based on these findings, clusters C1, C2, and C3 were renamed as “abortive reactivation,” “full reactivation,” and “pre-reactivation,” respectively, while the Zta-low C4 cluster and the single-treatment clusters also expressing low levels of Zta were renamed as “latent.” To fully characterize these EBV reactivation states, a comprehensive analysis of all detectable EBV gene expression was performed (FIGS. 5E-F). Compared to latent cells, nearly all of the 51 detected EBV genes and gene aggregates had increased expression in the full reactivation cluster, whereas only Zta was enriched in abortive reactivation. Of the cells that achieve a full or abortive EBV reactivation state, it was found that chidamide plus HDM201-treated cells preferentially exhibited full reactivation (C2), while most chidamide plus epirubicin-treated cells showed abortive reactivation (C1) (FIGS. 5F and 13G). To experimentally validate the observed full activation versus abortive reactivation phenotypes, the expression level of a latent EBV gene BGLF4 and EBV DNA copy number were measured. BGLF4 was lower in chidamide plus epirubicin-treated cells than that of chidamide plus Mdm2 inhibitors HDM201- or nutlin-3a-treated cells by RT-qPCR, consistent with the expression pattern of the gene aggregate including BGLF4 in the scRNA-seq dataset (FIGS. 5G-H). Additionally, an up to 25-fold increase in EBV DNA copy number was detected in cells treated with chidamide plus nutlin-3a but not with chidamide plus epirubicin (or other topoisomerase inhibitors) (FIGS. 5I and 13H). Together, the data show that different treatment can induce distinct EBV reactivation states. While chidamide plus nutlin3a is able to induce full EBV reactivation, chidamide plus epirubicin preferentially lead to an abortive EBV reactivation phenotype. Therefore, Zta induced by different treatments may have different biological functions for EBV reactivation, as Zta expression induced by chidamide plus epirubicin during abortive reactivation fails to induce the full suite of lytic genes and subsequent viral replication, despite its high level of expression.

### Example 7

### Chidamide Combined With Epirubicin Induces SUMO Modified Inactive Zta

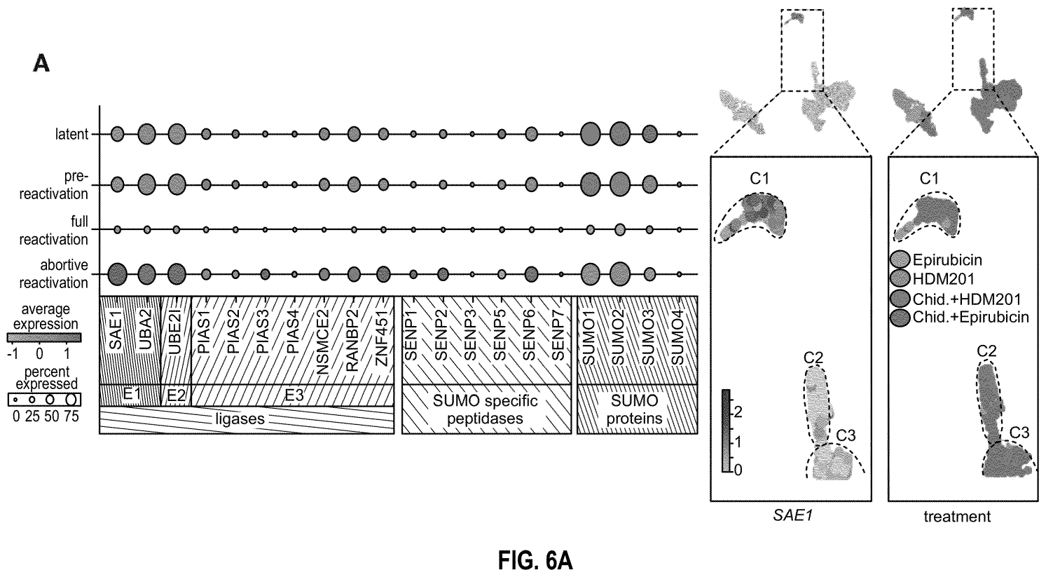

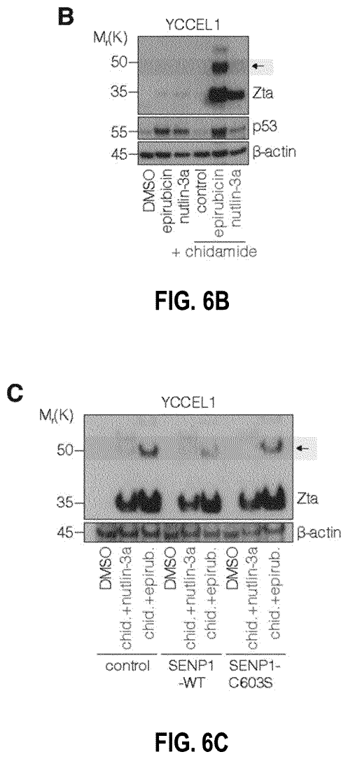

Many DNA damaging agents are known to induce SUMO modification pathway and SUMOylation of Zta has previously been shown to inhibit the ability of Zta to drive the EBV lytic cycle (Murata, T., et al. (2010). Journal of Biological Chemistry 285, 23925-23935.). It was thus hypothesized that chidamide combined with epirubicin but not chidamide combined with nutlin-3a may have different impacts on host cells SUMO modification pathway. To test this hypothesis, expression levels of 21 SUMO pathway components (FIG. 6A) in the single-cell dataset were assessed. Although the expression levels of most SUMO components are not affected by the treatments applied, interestingly, cells expressing the highest levels of SUMO E1-ligase component SAE1 (p_{adj}=7.4E−282) are in the C1 cluster, which has an abortive EBV reactivation state compared to all other cell clusters. Additionally, a moderate increase in UBE2I (p_{adj}=1.2E−83), which encodes the SUMO E2 UBC9, was also detected in the C1 cluster (FIG. 6B). These data indicate the SUMO pathway may be more active during abortive reactivation than during full reactivation or latency.

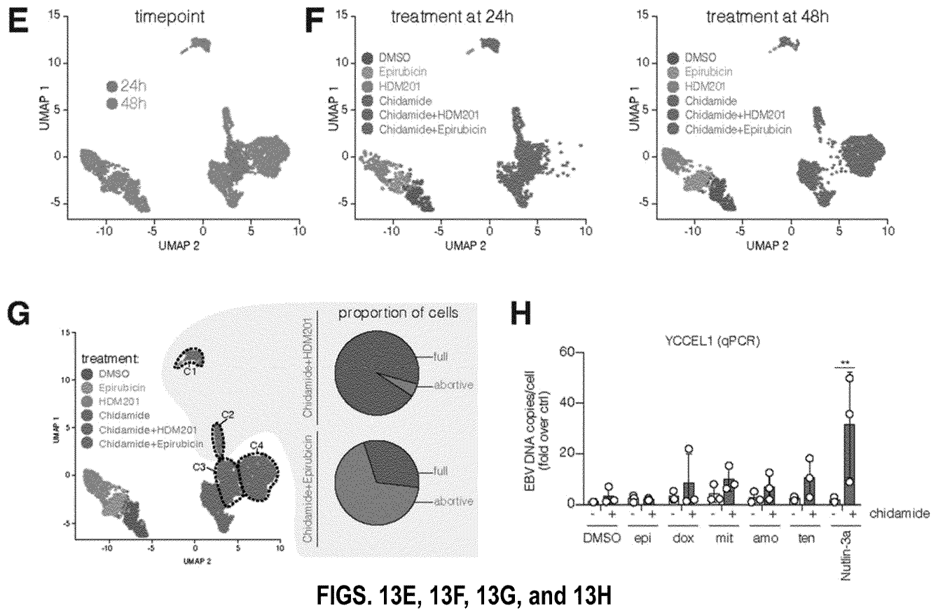

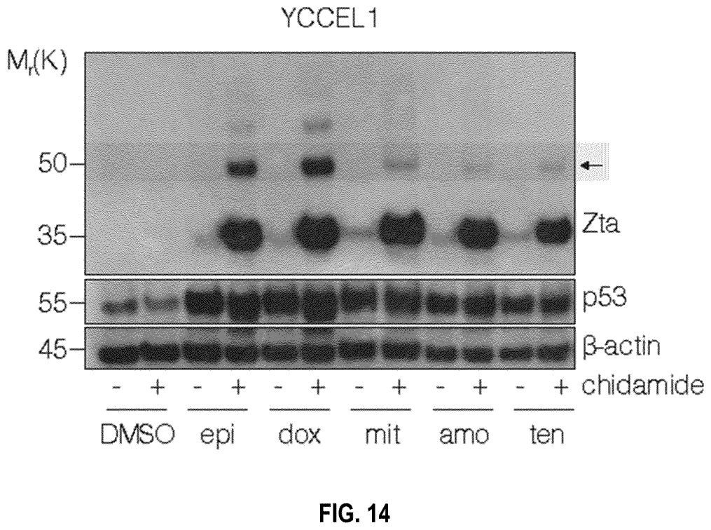

SUMOylation of Zta was examined using immunoblotting of cell lysates derived from DNSO, chidamide plus epirubicin or chidamide plus nutlin3a treated YCCEL1 cells. Unmodified Zta runs as a 35 kDa protein, and SUMOylated Zta is known to migrate at ˜47 kDa. In agreement with the hypothesis, a ˜47 kDa and ˜60 kDa protein ladder was specifically detected by an anti-Zta antibody in chidamide combined with epirubicin but not in chidamide combined with nutin3a treated YCCEL 1 cells. Under the same conditions, both treatments induced high levels of 35 kDa unmodified Zta (FIG. 6B). Similar to epirubicin, other topo II inhibitors such as doxorubicin, mitoxantrone, amonafide, and teniposide also induced modified 47 kDa Zta bands. Like epirubicin, doxorubicin also induced a detectable 60 kDa Zta (FIG. 14A). The pattern and the molecular weight shift of slow migrating Zta indicates they may be SUMOylated. To test this, a de-SUMOylating enzyme SENP1 or its enzyme dead mutant SENP1-C603S were expressed in YCCEL1 cells followed by chidamide plus nutlin-3a- or chidamide plus epirubicin-treatment. Incubation of SENP1 but not SENP1-C603S led to a decrease in the intensity of the 47 kDa-Zta band, consistent with this band corresponding to SUMOylated Zta (FIG. 6C). These data indicate that treatment with certain topoisomerase II inhibitors, combined with chidamide induces SUMO modification activity of the treated cells. As a result, some of the induced Zta protein is SUMO modified, which in turn inhibits its ability to activate the EBV lytic cycle. Hence, the ability of chidamide and epirubicin to induce SUMOylated Zta is a molecular mechanism through which this unique treatment combination can induce an abortive EBV reactivation phenotype. This principle is likely to apply to chidamide plus other Topo II inhibitors tested.

### Example 8

### Zta-Specific T Cells can Kill Latency-Reversed EBV-Positive Gastric Cancer Cells

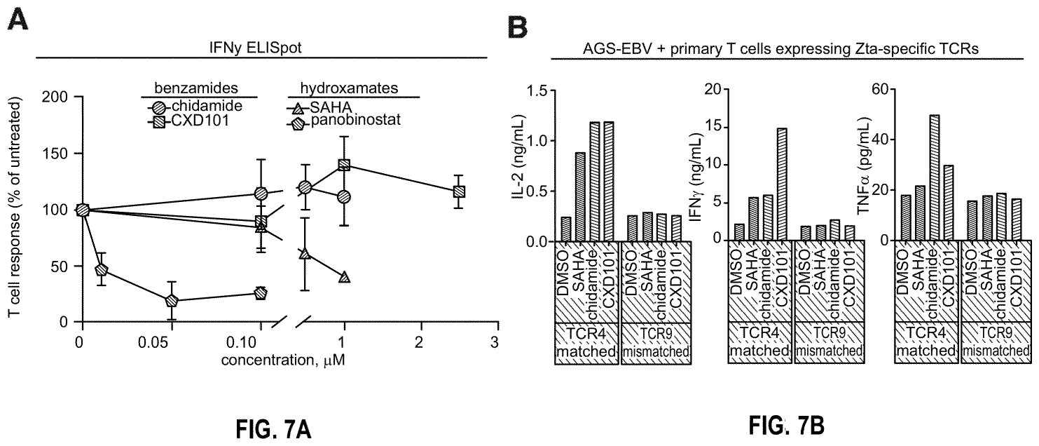

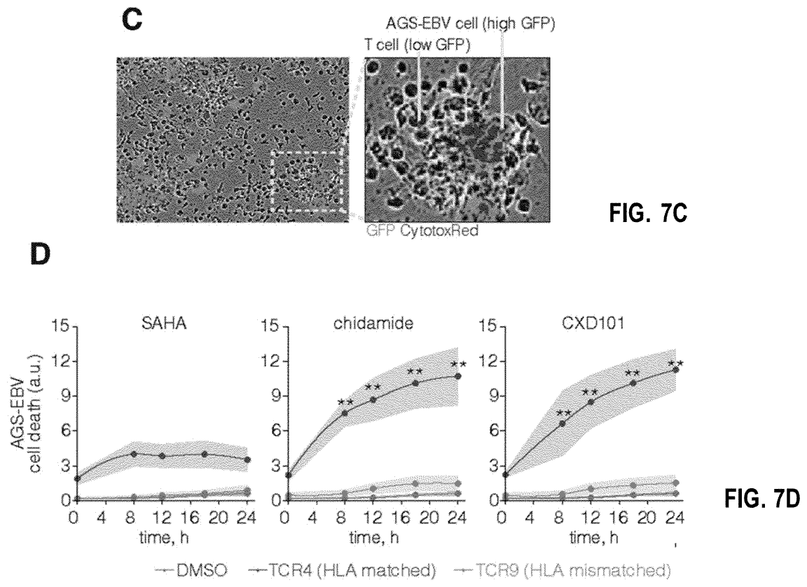

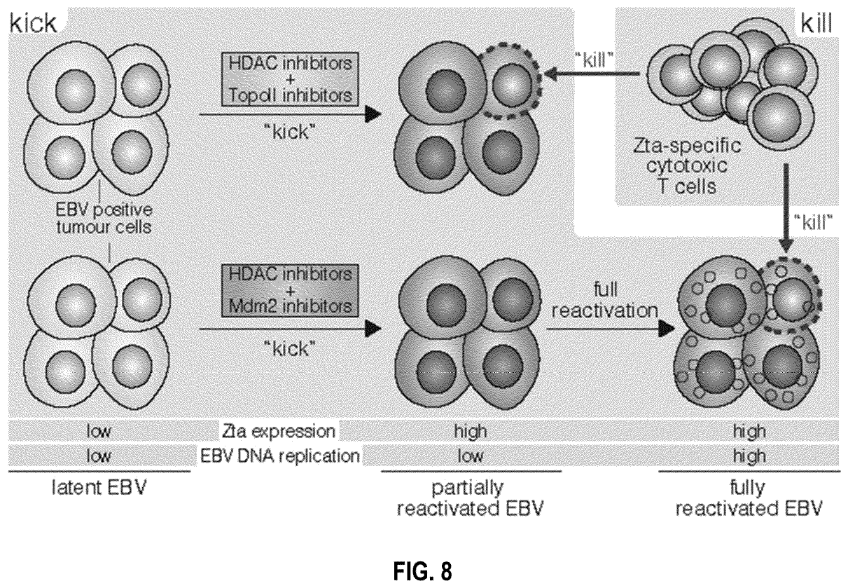

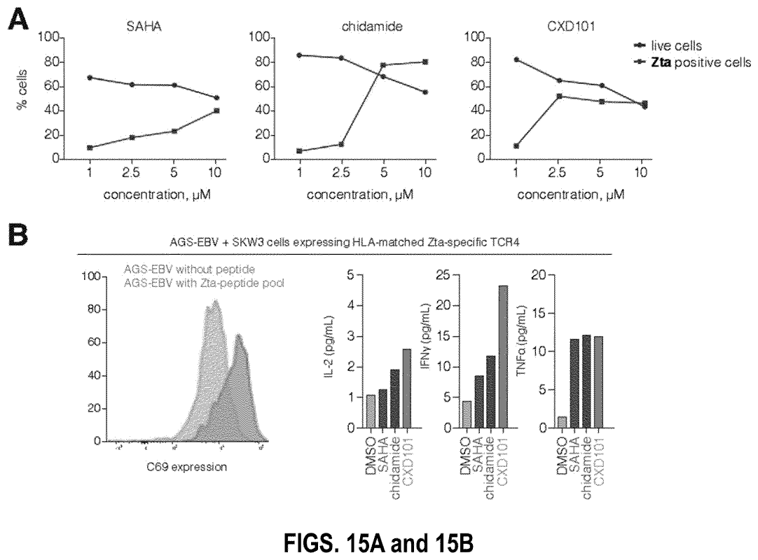

To be able to effectively “kick” EBV+GC cells into Zta+ abortive EBV reactivation lytic cycle presented us with an opportunity to “kill” the reactivated EBV+ cancer cells by harnessing the host's immune system, Zta specific T cells in particular as Zta is highly immunogenic (Pudney, V. A., et al. (2005). J Exp Med 201, 349-360) and many EBV seropositive individuals have Zta memory T cells. This study was set out to test whether HDACi have any direct impacts on CD8+ T cell responses to EBV viral antigens. Peripheral blood mononuclear cells (PBMCs) from an EBV+ healthy donor were stimulated with a pool of pre-determined EBV epitopes in the presence or absence of HDACi for 12 hours, and ex vivo EBV-specific CD8+ T cell responses were determined by an IFNγ ELISpot assay. Benzamide-based HDACi chidamide and CXD101 had little, if any, inhibitory effect on the T cell responses to EBV antigens, compared with hydroxamate-based HDACi panobinostat or SAHA over a range of doses tested (FIG. 7A). Panobinostat exhibited substantially greater inhibition of the T cell responses than SAHA, particularly at lower concentrations. In parallel, the optimal dose of each HDACi for AGS-EBV to minimize cell toxicity versus their ability to induce Zta expression was determined (FIG. 15A). To provide a proof of principle, whether reactivation of Zta by HDACi could sensitize tumor cells for recognition and killing by Zta-specific CD8+ T cells was subsequently tested. A co-culture system was developed using an HLA-A02: 01 restricted Zta-specific T cell receptor (TCR) generated by a single TCR cloning platform and an HLA-matched EBV latently-infected gastric cell line (AGS-EBV). The TCR gene was retrovirally transduced into a TCR-negative T cell line (SKW3) or primary human CD8+ T cells for functional readouts, including cytokine production and killing of the tumor cells. An HLA-matched artificially infected AGS-EBV cell line was used, which showed higher sensitivity to HDACi treatments, including SAHA, compared with naturally infected cell line YCCEL1 (FIG. 1C).

Given the high sensitivity of TCR recognition, it is plausible that even in latency, sufficient antigen ‘leaking’ might occur in AGS cells to trigger an immune response. However, the Zta-specific TCR-transduced SKW3 T cells did not exhibit detectable recognition (IL-2, IFNγ, and TNFα production) of AGS-EBV cells without HDACi treatment (FIG. 15B). Activation was only observed in the presence of HDACi, indicating that the AGS-EBV cells induce a T cell response similar to native EBV-infected gastric tumor cells, such as YCCEL1 cells. Finally, the functional analysis demonstrated that human primary CD8+ T cells transduced with the Zta-specific TCR (TCR4) were able to recognize HDACi-treated AGS-EBV cells, as shown by cytokine production (IL-2,IFNγ, and TNFα) (FIG. 7B). By contrast, cytokine production was absent in primary T cells transduced with HLA-mismatched clone TCR9 when incubated with HDACi-treated AGS-EBV cells (FIG. 7B). Importantly, using a fluorescent apoptotic marker (FIG. 7C), cell death of AGS-EBV cells incubated with primary T cells expressing HLA-matched Zta-specific TCR4 was analyzed. It was found that EBV reactivation with HDACi SAHA, chidamide, or CXD101 led to a dramatic increase in cell death after 8 h of co-culture, compared to DMSO control (FIG. 7D). Toxicity induced by T cells was more pronounced in AGS-EBV cells treated with benzamide-based HDACi chidamide and CXD101 compared to SAHA (P<0.01). Moreover, incubation of AGS-EBV cells with T cells expressing HLA-mismatched clone TCR9 had only a minor effect on cell death induction compared with HLA-matched clone TCR4 (FIG. 7D). The functional assays indicate that primary T cells transduced with Zta-specific TCRs can kill gastric cancer cells in an HDACi-dependent manner.

### Example 9

### Zta-Specific Primary T Cell Clones Kill HDACi-Treated AGS_rEBV Cells

During lytic infection, the EBV genes are expressed as a regulated cascade; the immediate early genes, followed by the early genes, and finally the late genes. The immediate early genes, BZLF1 and BRLF1, encode transactivator proteins Zta (BamHI Z Epstein-Barr virus Replication Activator), and Rta respectively. Zta is the most immunodominant antigen of EBV compared to other lytic stage proteins, and numerous CD8+ T cell epitopes presented by HLA-A, -B, and -C have been defined (Rist et al., J Virol, 89, 703-12 (2015)). Its recognition leads to a potent immune response consisting of cytokine production and T cell-mediated killing. Healthy carriers sustain high frequencies of Zta-specific memory T cells, and that these potentially help in controlling virus spread during spontaneous reactivation during persistent infection.

The EBV Zta-specific TCRs that were cloned from the two healthy donors were characterized in terms of their HLA-restriction and their peptide-specificities. Table 3 shows a summary of these characteristics. As the overlapping epitopes are presented by several different HLA alleles, they could be exploited therapeutically for the design of treatments. These TCRs comprised up to 21% of sequences in two healthy donors and were successfully expressed in a TCR-deficient T-cell line and in primary T-cells. Transduced T-cells upregulated the early activation marker CD69 and secreted TNF-α and IFN-γ upon stimulation, demonstrating preserved functional ability of the cloned TCRs.

Characterization of a previously undiscovered TCR from one healthy donor revealed its ability to recognize antigen presented on two different autologous HLA alleles, HLA-A*02:01 and HLA-B*35:01. Such cross-reactivity to HLA molecules within the same donor has not been described in literature and supports the use of T-cell therapy for patients carrying either, or both, HLA types.

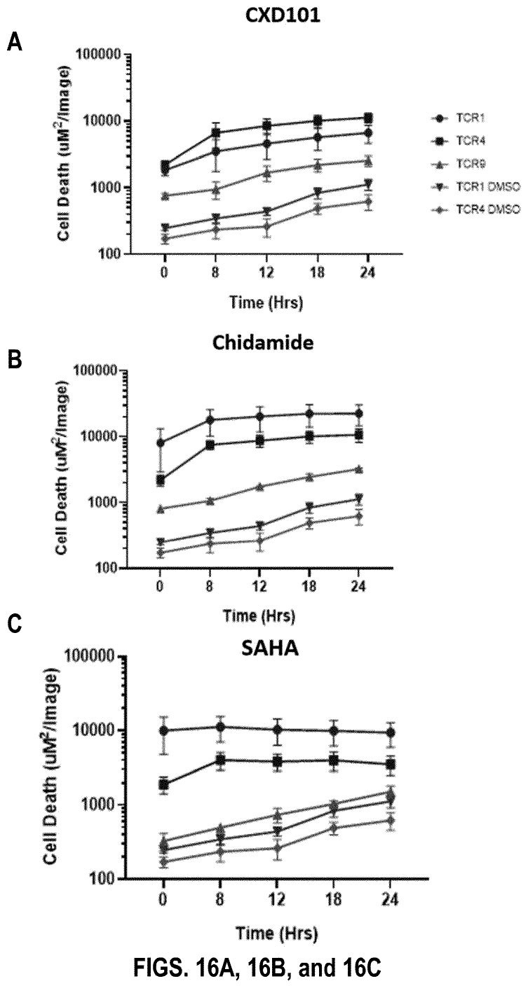

Moreover, functional studies indicated that Zta-specific TCR-transduced primary T-cells could recognize and mediate killing of EBV-associated gastric carcinoma cells after virus latency reversal with HDAC inhibitors (FIGS. 16A-C). FIGS. 16A-C show that Zta-specific primary T cell clones kill HDACi-treated AGS_rEBV cells. AGS_rEBV were treated with HDACi (FIG. 16A: CXD101; FIG. 16B: Chidamide; FIG. 16C: SAHA) or DMSO for 48 hours before co-culturing with primary T cell clones for 24 hours. Cell death is represented as uM2/per image, which represents the area of cells with fluorescence overlap of very high GFP (EBV-positive gastric cells) with red apoptosis marker in each replicate. Data shown is the mean of 4 replicates from the same experiment.

TABLE 3
Summarized characteristics of cloned EBV Zta peptide-specific TCRs

| | | | | | SEQ | | SEQ | |
|---|---|---|---|---|---|---|---|---|
| | | | | | ID | Mapped | ID | HLA- |
| Donor | TCR | TCR V/J genes^{§} and CDR3 | | | NO | Peptide | NO | restriction |
| 1 | 4 | TRAV20*02 | TRAJ24*02 | CAFFSWGKLQF | 37 | EPLPQGQ | 49 | A*02:01 |
| | | | | | | LTAY | | |
| | | TRBV3- | TRBJ1- | CASSQSPGTGV | 38 | LPQGQLT | 50 | B*35:01 |
| | | 1*01 | 2*01 | GYTF | | AYHV | | |
| 2 | 9 | TRAV13- | TRAJ42*01 | CAASKEGGGSQ | 39 | DSELEIK | 51 | B*18:01 |
| | | 1*02 | | GNLIF | | RY | | |
| | | TRBV20- | TRBJ2- | CSARDRGEHPP | 40 | | | |
| | | 1*02 | 1*01 | NDQFF | | | | |
| 2 | 16 | TRAV13- | TRAJ12*01 | CAEKGWDSSYK | 41 | IKRYKNR | 52 | A*23:01 |
| | | 2*01 | | LIF | | V | | |
| | | TRBV30*02 | TRBJ1- | CAWVDGVLDGY | 42 | | | |
| | | | 2*01 | TF | | | | |
| 2 | 34 | TRAV29*01 | TRAJ16*01 | CAASEGGQKLL | 43 | RYKNRVA | 53 | A*23:01 |
| | | | | F | | S | | |
| | | TRBV7- | TRBJ2- | CASSAPNSDSA | 44 | | | |
| | | 9*03 | 4*01 | KNIQYF | | | | |
| 2 | 131 | TRAV26- | TRAJ47*01 | CILRDGVGYGN | 45 | DSELEIK | 54 | B*18:01 |
| | | 2*01 | | KLVF | | RY | | |
| | | TRBV9*02 | TRBJ2- | CASEDRGGTDT | 46 | | | |
| | | | 3*01 | QYF | | | | |
| 2 | 132 | TRAV14*01 | TRAJ52*01 | CAMREGSGVGT | 47 | CDSELEI | 55 | B*18:01 |
| | | | | SYGK | | KR | | |
| | | TRBV29- | TRBJ2- | CSAAGTSGSGE | 48 | | | |
| | | 1*01 | 5*01 | TQYF | | | | |

^{§}The sequences of the TCR V/J genes listed in this table are available from the IMGT database (http://www.imgt.org).

### Discussion

This disclosure shows that a combination of the benzamide-based HDACi (e.g., chidamide) and the topoisomerase II inhibitor (e.g., epirubicin) (or an Mdm2 inhibitor) can efficiently reactivate expression of the immediate-early EBV protein Zta, whilst minimizing risk of uncontrolled EBV infection. Expression of the highly immunogenic protein Zta in up to 50% of EBV-infected cancer cells treated with these drugs provides an opportunity to eliminate EBV-infected cancer cells via T cell-mediated killing by existing Zta-specific memory T cells. The results demonstrate the “kick and kill” strategy as an effective cancer therapy for treating virus-associated cancers.

### Wild Type p53 Activity Controls EBV Reactivation by Benzamide-Based HDACi

p53 interacts with over 100 cellular proteins, many of which are involved in epigenetic regulation. The finding that benzamide-based HDACi can synergize with p53-enhancing agents to reverse EBV latency with high efficiency indicates that there is functional cross-talk between class I HDAC and p53 in controlling EBV latency. This is supported by previous evidence of physical and functional interactions between p53 and class I HDACs. p53 was one of the first non-histone proteins identified as a substrate of acetyltransferase p300 (Gu, W., et al. Cell 90, 595-606 (1997).), and acetylation enhances the transcriptional activity of p53. By binding and de-acetylating p53, HDAC1 represses p53's transcriptional activity (Luo, J., et al. (2000). Nature 408, 377-381.). HDAC8, another class I HDAC, also binds and inhibits p53 function (Yu, X., et al. (2020). The Journal of Investigative Dermatology, 27 Feb. 2020, 140 (10): 2009-2022.e4). HDAC2 can inhibit the DNA binding and transcriptional activities of p53 without altering p53 stability or acetylation (Harms, K. L., and Chen, X. (2007). Cancer Research 67, 3145-3152.). Moreover, class I HDACs also regulate p53 activities through their ability to regulate Mdm2. Thus, class I-specific HDACi are likely to enhance the transcriptional activity of p53 and may synergize with p53 agonists, such as nutlin, potentially explaining the observations.

p53 is mutated in around 50% of human cancers, but its mutation frequency varies from ˜5% in leukemia to ˜95% in ovarian cancers, raising the question of how the tumor-suppressive function of p53 is overcome in tumors with wild type TP53 (p53) genes. Cancer-causing viruses can have mechanisms to bypass p53's tumor suppressive function. For example, oncoprotein E6 of HPV binds to p53 and targets p53 for degradation. As a result, p53 is rarely mutated in HPV-infected cervical or head and neck cancers. Interestingly, p53 is rarely mutated in most EBV-containing-epithelial cancers (compared to ˜50% of B cell lymphomas). The findings that wild type p53 activity is required for the ability of HDACi to reverse EBV latency and that the extent of p53 activity controls efficacy, indicating that maintaining wild type p53 in host cells may be crucial for the long-term survival of EBV, explaining low levels of p53 mutation. The intimate link between p53 and class I HDAC in control of EBV latency also indicates that EBV might bypass the requirement for p53 mutation in tumorigenesis by altering the epigenome of the host cells. Consistent with this, EBV+ epithelial cancer cells, gastric cancer cells, in particular, tend to have highly methylated genomes.

### Elevated SUMO-Modification may Lead to Abortive EBV Reactivation

The expression and activity of SUMO-modifying enzymes are responsive to various cellular stress signals, in particular, DNA damage, which induces global changes in SUMOylation, as well as SUMOylation of key replication and repair proteins at the site of double-stranded breaks. The topoisomerase II inhibitor epirubicin induces p53 through its ability to induce a cellular response to DNA damage signals, whereas HDM201 induces p53 by enhancing its protein stability without causing DNA damage. These distinct routes by which epirubicin and HDM201 induce p53—DNA damage-dependent and -independent pathways, respectively—may explain why treatment including epirubicin induces SAEl expression whereas HDM201 does not.

In many cells treated with chidamide plus epirubicin, high expression of Zta, without increases in other lytic genes, was observed. Previous studies have shown that Zta can be modified by SUMO1-3 at K12; this SUMOylation causes Zta to associate with epigenetic repressor complexes, including HDAC3 and HDAC7, reducing the activity of Zta-responsive promoters and attenuating the ability of Zta to drive the lytic cycle. The SUMO E1 ligase, a protein heterodimer formed by SAE1 and UBA2, plays a critical role in activating the available SUMO pool for protein modification (Rabellino, A., et al. (2017). Cancer Research 77, 1542-1547.). Hence, the finding of strongly enhanced SAEl expression in chidamide plus epirubicin-treated cells, as well as evidence of Zta SUMOylation, indicates that SUMOylation of Zta is the mechanism underlying abortive reactive in these cells.

### Harnessing the Host Immune System to Achieve Kick and Kill of Cancer Cells

Clinical trials of kick and kill for HIV have encountered two major barriers. First, the kicking efficiency is below expectation, and second, the HDACi used for latency reversal often inhibit T cell function. Both these limitations are addressed in these examples: benzamide-based HDACi are more potent than hydroxamate-based HDACi to reactivate EBV latency, and they also have lower toxicity for CD4 and CD8 T cell function (FIG. 7). Furthermore, treatments triggering abortive reactivation can help to achieve “controlled kicking,” i.e., maximal efficiency of the ‘kick’ with minimal toxicity associated with viral reactivation. Consistent with the notion of harnessing host immune system to achieve specific killing of virus-infected cells, a recent study demonstrated increased MHC-II expression in EBV-positive gastric cancer compared with EBV-negative tumors (Ghasemi, F., et al. Scientific Reports volume 10, 14786 (2020)). These results indicate that Zta-specific CD8+ T cells generated through natural infection or adaptive TCR-based immunotherapy would achieve selective and efficient killing of reactivated EBV-associated gastric tumor cells in vivo.

In summary, the foregoing examples present a novel cancer treatment approach through the identification of a defined combination of clinically applied drugs (e.g., chidamide and epirubicin) to achieve a controlled effective “kick” and combined with a specific and effective “killing” of EBV-positive gastric cancer cells by harnessing the host's immune system, through, e.g., Zta-specific CD8 T cells.

1. A method of reactivating a latent Epstein-Barr virus (EBY) in a cell infected with the EBY, comprising contacting the cell with a benzamide-based histone deacetylase (HDAC) inhibitor, wherein the benzamide-based HDAC inhibitor increases a level of expression or activity of an EBY-associated protein in the EBY-positive cancer cell.

2. A method of killing an EBY-positive cancer cell in a subject infected with the EBY, comprising administering to the subject an effective amount of a benzamide-based HDAC inhibitor, wherein the benzamide-based HDAC inhibitor increases a level of expression or activity of an EBY-associated protein in the EBY-positive cancer cell.

3. A method of eliciting or enhancing an immune response against an EBY-positive cancer cell in a subject infected with the EBY, comprising administering to the subject an effective amount of a benzamide-based HDAC inhibitor, wherein the benzamide-based HDAC inhibitor increases a level of expression or activity of an EBY-associated protein in the EBY-positive cancer cell.

4. A method of treating a subject having cancer associated with EBY infection, comprising administering to the subject an effective amount of a benzamide-based HDAC inhibitor, wherein the benzamide-based HDAC inhibitor increases a level of expression or activity of an EBY-associated protein in an EBY-positive cancer cell.

5. The method of claim 1, wherein the cell is an EBY-positive cancer cell.

6. The method of claim 2, wherein the EBY-positive cancer cell is an EBY-positive gastric cancer cell.

7. The method of claim 4, wherein the cancer is gastric cancer.

8. The method of claim 1, wherein the EBY-associated protein is transcription factor Zta.

9. The method of claim 1, wherein the benzamide-based HDAC inhibitor comprises any one of chidamide, CXD101, entinostat, mocetinostat, and combinations thereof.

10. The method of claim 1, wherein the benzamide-based HDAC inhibitor comprises chidamide.

11. The method of claim 1, further comprising contacting the cell with a second agent.

12. The method of claim 2, further comprising administering to the subject a second agent.

13. The method of claim 11, wherein the second agent comprises a topoisomerase inhibitor.

14. The method of claim 13, wherein the topoisomerase inhibitor comprises any one of epirubicin, doxorubicin, mitoxantrone, amonafide, teniposide, and combinations thereof.

15. The method of claim 13, wherein the topoisomerase inhibitor comprises epirubicin.

16. The method of claim 11, wherein the second agent comprises an Mdm2 inhibitor.

17. The method of claim 16, wherein the Mdm2 inhibitor comprises nutlin-3a, HDM201, or a combination thereof.

18. The method of claim 11, wherein the second agent comprises an anti-cancer agent.

19. The method of claim 2, further comprising administering to the subject a lymphocyte transduced with a recombinant T cell receptor (TCR).

20. The method of claim 19, wherein the recombinant TCR comprises a Zta-specific TCR.

21. The method of claim 19, wherein the recombinant TCR-transduced lymphocyte shows reactivity to the transcriptional factor Zta or a fragment thereof.

22. The method of claim 19, wherein the recombinant TCR binds specifically to the transcriptional factor Zta or a fragment thereof.

23. The method of claim 19, wherein the recombinant TCR comprises an amino acid sequence having at least 90% sequence identity to an amino acid sequence of SEQ ID NOs: 37-48 or comprises an amino acid sequence of SEQ ID NOs: 37-48.

24. The method of claim 19, wherein the recombinant TCR binds specifically to an antigen comprising an amino acid sequence of SEQ ID NOs: 49-53 and 55.

25. The method of claim 18, wherein the lymphocyte comprises a CDS+ T cell or a CD4+ T cell.

26. The method of claim 2, further comprising administering to the subject an EBY vaccine and optionally an adjuvant.

27. The method of claim 2, wherein the benzamide-based HDAC inhibitor is administered orally, topically, intravenously, intraperitoneally, intramuscularly, intralesionally, intrathecally, intranasally, subcutaneously, parenterally, transmucosally, sublingually, in controlled release, in delayed release, or as a suppository.

28. The method of claim 12, wherein the second agent is administered to the subject before, after, or concurrently with the benzamide-based HDAC inhibitor.

29. The method of claim 2, wherein the subject is a mammal.

30. The method of claim 29, wherein the subject is a human.

31. A composition for eliciting or enhancing an immune response against an EBY-positive cancer cell in a subject infected with the EBY, comprising: (i) benzamide-based HDAC inhibitor; (ii) a topoisomerase inhibitor or an Mdm2 inhibitor; and (iii) optionally a pharmaceutically acceptable carrier.

32. The composition of claim 31, wherein the benzamide-based HDAC inhibitor
comprises any one of chidamide, CXD101, entinostat, mocetinostat, and combinations thereof.

33. The composition of claim 32, wherein the topoisomerase inhibitor comprises any one of epirubicin, doxorubicin, mitoxantrone, amonafide, teniposide, and combinations thereof.

34. The composition of claim 31, wherein the Mdm2 inhibitor comprises nutlin-3a, HDM201, or a combination thereof.

35. The composition of claim 31, comprising chidamide, epirubicin, and optionally the pharmaceutically acceptable carrier.

36. The composition of claim 31, wherein the composition is an immunogenic composition optionally comprising a pharmaceutically acceptable diluent, vehicle, one or more immunological adjuvants, or combinations thereof.

37. A kit for for eliciting or enhancing an immune response against an EBY-positive cancer cell in a subject infected with the EBY, comprising: (i) benzamide-based HDAC inhibitor; (ii) a topoisomerase inhibitor or an Mdm2 inhibitor; and (iii) optionally a pharmaceutically acceptable carrier.

38. The kit of claim 37, wherein the benzamide-based HDAC inhibitor comprises any one of chidamide, CXDlOl, entinostat, mocetinostat, and combinations thereof.

39. The kit of claim 37, wherein the topoisomerase inhibitor comprises any one of epirubicin, doxorubicin, mitoxantrone, amonafide, teniposide, and combinations thereof.

40. The kit of claim 37, wherein the Mdm2 inhibitor comprises nutlin-3a, HDM201, or a combination thereof.

41. The kit of claim 37, comprising chidamide, epirubicin, and optionally the pharmaceutically acceptable carrier.

42. A TCR or antigen-binding fragment thereof, comprising an amino acid sequence having at least 90% sequence identity to an amino acid sequence of SEQ ID NOs: 37-48 or comprises an amino acid sequence of SEQ ID NOs: 37-48.

43. The TCR or antigen-binding fragment thereof of claim 42, wherein the TCR or antigen-binding fragment thereof binds specifically to the transcriptional factor Zta or a fragment thereof.

44. The TCR or antigen-binding fragment thereof claims claim 42, wherein the recombinant TCR binds specifically to an antigen comprising an amino acid sequence of SEQ ID NOs: 49-53 and 55.

45. A nucleic acid comprising a polynucleotide sequence that encodes the TCR or antigen-binding fragment thereof of claim 42.

46. A vector comprising the nucleic acid of claim 45.

47. The vector of claim 46, comprising a retroviral vector or a lentiviral vector.

48. A cell comprising the nucleic acid of claim 45.

49. The cell of claim 48, wherein the cell comprises an immune cell.

50. The cell of claim 49, wherein the immune cell comprises a lymphocyte.

51. A composition comprising the TCR or antigen-binding fragment claims of claim 42.
